(19) United States
(12) Patent Application Publication
(10) Pub. No.: US 2025/0145692 A1
(43) Pub. Date: May 8, 2025

(54) CHIMERA MOLECULES FOR SARS-COV-2 AND METHODS OF USE

(71) Applicants: Shaoxiao Wang, Wynnewood, PA (US); Simon Wang, Wynnewood, PA (US); Yi Wang, Wynnewood, PA (US)

(72) Inventors: Shaoxiao Wang, Wynnewood, PA (US); Simon Wang, Wynnewood, PA (US); Yi Wang, Wynnewood, PA (US)

(21) Appl. No.: 18/935,511

(22) Filed: Nov. 2, 2024

Related U.S. Application Data
(60) Provisional application No. 63/595,999, filed on Nov. 3, 2023.

(51) Int. Cl. C07K 16/10; A61K 38/00; A61K 39/00; C07K 19/00; C12N 9/10

(52) U.S. Cl. CPC C07K 16/1003; C07K 19/00; C12N 9/104; A61K 38/00; A61K 39/00; C07K 2317/569

(57) ABSTRACT

The subject matter described herein is directed to a chimera molecule such as a polypeptide or protein chimera useful for treating or preventing a viral infection or reducing the severity, incidence, or transmissibility of a viral infection, to nucleic acid molecules encoding the polypeptide or protein chimera, and to pharmaceutical compositions containing them. Also, methods of generating such antiviral polypeptide or protein chimera, of generating nucleic acid molecules encoding the antiviral polypeptide or protein chimera, and of generating pharmaceutical compositions containing the same, and methods of using the same for treatment or prevention of a SARS-CoV-2 infection, or to reduce the severity, incidence, or transmissibility of a of a SARS-CoV-2 infection, are described.

### CROSS-REFERENCE TO RELATED APPLICATION

This application claims priority to U.S. Provisional Application No. 63/595,999, filed Nov. 3, 2023, the entire disclosure of which is specifically incorporated herein by reference.

### INCORPORATION OF MATERIAL OF XML SEQUENCE LISTING BY REFERENCE

The sequence listing submitted herewith as a XML file named “1425001SEQUENCELISTINGXML” created Nov. 1, 2023, which is 99,000 bytes in size, is hereby incorporated by reference in its entirety.

### FIELD OF THE DISCLOSURE

This disclosure relates to a chimera molecule such as a polypeptide or protein chimera useful for treating or preventing a viral infection or reducing the severity, incidence, or transmissibility of viral infection, to nucleic acid molecules encoding the polypeptide or protein chimera, and pharmaceutical compositions containing them. Methods of generating such antiviral polypeptide or protein chimera, nucleic acid molecules encoding the antiviral polypeptide or protein chimera, and pharmaceutical compositions containing the same, and methods of using the same for treatment or prevention of a viral infection, or to reduce the severity, incidence, or transmissibility of a viral infection are provided. Particularly, this disclosure relates to a polypeptide or a protein chimera, nucleic acids molecules encoding the polypeptide or the protein chimera, pharmaceutical compositions containing the same, and therapeutic methods for the treatment or prevention of a SARS-CoV-2 infection, and methods for reducing the severity, incidence, or transmissibility of SARS-CoV-2.

### BACKGROUND OF THE DISCLOSURE

Since the beginning of 2020, SARS-CoV-2 has infected more than 7 hundred million people worldwide and caused nearly 7 million deaths. These numbers are still rising up while many vaccines have been developed and administered to most people with multiple dosages. In the meantime, all kinds of treatments have been intensively studied and some have been clinically used to treat patients and shown promising results. However, all these vaccines and drugs cannot totally stop the pandemic, and more effective therapeutic strategies need to be explored and discovered.

SARS-CoV-2 is a member of a large family of viruses called coronaviruses. Its RNA genome encodes at least 29 proteins, four of which are structural proteins: the spike (S), membrane (M), envelope (E) and nucleocapsid (N) proteins. The M, E, and N proteins are critical for viral particle assembly and release, whereas the S protein is responsible for viral binding and entry into host cells through their surface protein human angiotensin converting enzyme 2 (ACE2) as an entry receptor. ACE2 is a cell surface receptor predominant in the lung, heart, and kidney. SARS-CoV-2 is mostly transmissible through large respiratory droplets, directly infecting cells of the upper and lower respiratory tract, especially nasal ciliated and alveolar epithelial cells.

Following host cell binding, the virus fuses with a cell membrane and then enters into the cell. For many coronaviruses, including SARS-CoV-2, host cell binding alone is insufficient to facilitate membrane fusion, which also requires S-protein priming or cleavage by host cell proteases or transmembrane serine proteases. Unlike other coronaviruses, SARS-CoV-2 possesses an unique furin-like cleavage site in the S-protein, which is therefore cleaved into the S1 and S2 subunits by ubiquitously expressed furin-like proteases, indicating that S-protein priming at this cleavage site may contribute to the widened cell tropism and enhanced transmissibility of SARS-CoV-2. Once the nucleocapsid is released into the cytoplasm of the host cell, the RNA genome is replicated and translated into structural and accessory proteins, resulting in multiple virus assembly. Vesicles containing the newly formed viral particles are then transported to and fuse with the plasma membrane, releasing them to infect other host cells in the same fashion.

Thus, therapeutic agents that inhibit virus assembly as provided herein can beneficially interfere in the process of SARS-CoV2 infection of other host cells, and hence aid in the treatment or prevention of a SARS-CoV-2 infection, as well as reduce the severity, incidence, or transmissibility of SARS-CoV-2.

### SUMMARY OF THE DISCLOSURE

In one aspect, the disclosure provides a chimera molecule having the formula:

Ab-L-P

wherein Ab is an antibody that specifically binds to a Severe Acute Respiratory Syndrome coronavirus 2 (SARS-CoV-2) Spike (S) protein antigen, the Ab is conjugated to L; L is a linker or a bond covalently or non-covalent bound to Ab and to P; and P is a ubiquitin E3 ligase.

In another aspect of the above embodiment, Ab is a nanobody that specifically binds to a Severe Acute Respiratory Syndrome coronavirus 2 (SARS-CoV-2) Spike (S) protein antigen, and P is a E2 recruiting domain of the ubiquitin E3 ligase.

In one aspect of any one of the above embodiments, the chimera molecule is a polypeptide or protein chimera that specifically binds to a Severe Acute Respiratory Syndrome coronavirus 2 (SARS-CoV-2) Spike (S) protein antigen, and is capable of mediating Spike (S) protein degradation.

In one aspect of any one of the above embodiments, the Spike (S) protein specific nanobody is selected from the group consisting of Ty1 and H11-H4

In one aspect of any one of the above embodiments, Ab has an amino acid sequence selected from any one of SEQ ID NOs: 11, 64, 22, 66, 67, or 70, or an amino acid sequence having at least 85% sequence identity to the amino acid sequence of any one of SEQ ID NOs: 8, 11, 64, 22, 66, 67, or 70

In one aspect of the above embodiment, Ab has an amino acid sequence encoded by a nucleic acid sequence selected from any one of SEQ ID NOs: 4, 17, 47, 63, 56, or 65, or an amino acid sequence encoded by a nucleic acid sequence having at least 85% sequence identity to the nucleic acid sequence of any one of SEQ ID NOs: 4, 17, 47, 63, 56, or 65

In one aspect of any one of the above embodiments, P has an amino acid sequence selected from any one of SEQ ID NOs: 12, 32, or 69, or an amino acid sequence having at least 85% sequence identity to the amino acid sequence of any one of SEQ ID NOs: 12, 32, or 69.

In one aspect of the above embodiment, P has an amino acid sequence encoded by a nucleic acid sequence selected from any one of 5, 27, or 68, or an amino acid sequence encoded by a nucleic acid sequence having at least 85% sequence identity to the nucleic acid sequence of any one of SEQ ID NOs: 5, 27, or 68.

In one aspect of any one of the above embodiments, Ab has an amino acid sequence selected from any one of SEQ ID NOs: 11, 22, 64, 66, 67, 70, and P has an amino acid sequence selected from any one of SEQ ID NOs: 12, 32, or 69.

In one aspect of the above embodiment, Ab has an amino acid sequence encoded by a nucleic acid sequence selected from any one of SEQ ID NOs: 4, 17, 47, 56, 63, or 65, and P has an amino acid sequence encoded by a nucleic acid sequence selected from any one of 5, 27, or 68.

In one aspect of any one of the above embodiments, the chimera molecule is a polypeptide or a protein chimera. In particular, the polypeptide or protein chimera has a sequence selected from any one of SEQ ID NOs: 8, 13, 20, 23, 30, 33, 39, 41, 48, 49, 57, or 58, or an amino acid sequence having at least 85% sequence identity to the amino acid sequence of any one of SEQ ID NOs: 8, 13, 20, 23, 30, 33, 39, 41, 48, 49, 57, or 58.

In another aspect of any one of the above embodiments, the chimera molecule inhibits, blocks or reduces viral assembly. In particular, the chimera molecule inhibits, blocks or reduces SARS-CoV-2 viral assembly.

In one aspect, the present disclosure provides a nucleic acid molecule encoding the polypeptide or protein chimera of any one of the above embodiments. In particular, the polypeptide or protein chimera has an amino acid sequence encoded by a nucleic acid sequence selected from any one of SEQ ID NOs: 1, 6, 15, 18, 25, 28, 35, 37, 43, 44, 52, or 53, or an amino acid sequence encoded by a nucleic acid sequence having at least 85% sequence identity to the nucleic acid sequence of any one of SEQ ID NOs: 1, 6, 15, 18, 25, 28, 35, 37, 43, 44, 52, or 53.

In another aspect, the present disclosure provides a composition containing a therapeutically effective amount of a chimera molecule of any one of the above embodiments, or a polypeptide or a protein chimera of any one of the above embodiments, in which the therapeutically effective amount is an amount sufficient to inhibit, block or reduce viral assembly; and at least one pharmaceutically acceptable excipient. In particular, the therapeutically effective amount is an amount of a chimera molecule or a polypeptide or protein chimera effective to inhibit, block or reduce SARS-CoV-2 viral assembly.

In another aspect, the present disclosure provides a method of treating a viral infection by administering to a subject in need thereof, a therapeutically effective amount of a chimera molecule of any one of the above embodiments, or a polypeptide or a protein chimera of any one of the above embodiments.

In another aspect, the present disclosure provides a method of preventing, reducing severity, reducing incidence, or reducing transmissibility of a SARS-CoV-2 infection by administering a composition containing a chimera molecule, or polypeptide or a protein chimera of any one of the above embodiments.

Another aspect of the disclosure is a method of making a chimera molecule of any one of the above embodiments, or a polypeptide or protein chimera of any one of the above embodiments.

Another aspect of the disclosure is a kit or article of manufacture containing a polypeptide or a protein chimera of any one of the above embodiments, and a package insert or label indicating that the polypeptide or protein chimera is useful for or can be used to treat a SARS-CoV-2 infection.

Another aspect of the disclosure is a kit or article of manufacture containing a polypeptide or a protein chimera of any one of the above embodiments, that specifically binds to a Severe Acute Respiratory Syndrome coronavirus 2 (SARS-CoV-2) Spike (S) protein antigen, and a package insert or label indicating that the polypeptide or protein chimera is useful for or can be used to prevent, reduce severity, reduce incidence, or reduce transmissibility of a SARS-CoV-2 infection.

### BRIEF DESCRIPTION OF THE DRAWINGS

FIG. 1 shows SARS-CoV-2 infection and proliferation.

FIG. 2 shows the ubiquitin-proteasome system (UPS).

FIG. 3A shows natural antibodies.

FIG. 3B shows antibody fragments.

FIG. 4 shows degradation of spike protein mediated by the protein chimera.

FIG. 5 is a Western blot showing detection of the effect of different protein chimeras degrading spike protein.

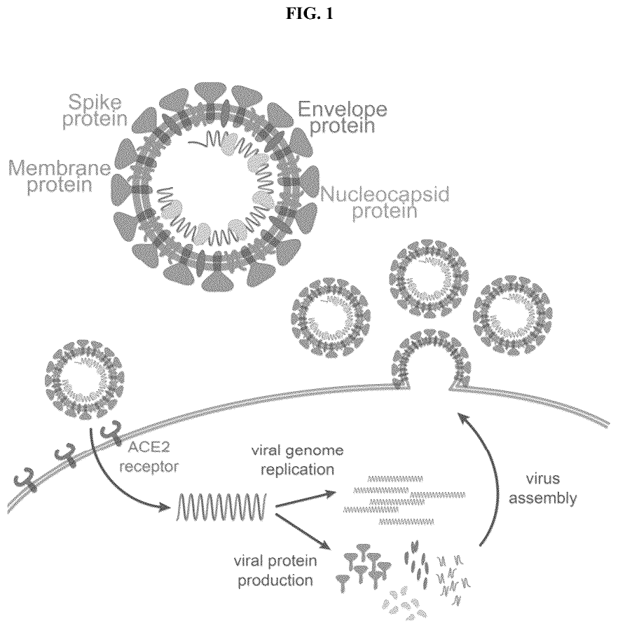

### DEFINITIONS

In order to facilitate understanding of the examples provided herein, certain frequently occurring methods and/or terms will be defined herein.

It must be noted that as used herein and in the appended claims, the singular forms “a”, “an” and “the” include plural references unless the context clearly dictates otherwise. Furthermore, the terms “a” (or “an”), “one or more,” and “at least one” can be used interchangeably herein. The terms “comprising,” “including,” “having,” and “constructed from” can also be used interchangeably. The term “comprising” is intended to include examples encompassed by the terms “consisting essentially of” and “consisting of” The term “consisting essentially of” is intended to include the embodiments or elements presented therein, whether explicitly set forth or not. Throughout this specification and claims, the word “comprise,” or variations such as “comprises” or “comprising,” will be understood to imply the inclusion of a stated integer or group of integers but not the exclusion of any other integer or group of integers.

As used herein, the term “about” means+/−10% of the recited value.

The terms “adenovirus vector” and “adenoviral vector” are used interchangeably and refer to a genetically-engineered adenovirus that is designed to insert a polynucleotide of interest (e.g., a polynucleotide encoding a polypeptide or protein chimera for a SARS-CoV-2 spike antigen as described herein) into a eukaryotic cell, such that the polynucleotide is subsequently expressed. Examples of adenoviruses that can be used as a viral vector include those having, or derived from, the serotypes Ad2, Ad5, Ad11, Ad12, Ad24, Ad26, Ad34, Ad35, Ad40, Ad48, Ad49, Ad50, Ad52 (e.g., RhAd52), Ad59 (e.g., RhAd59), and Pan9 (also known as AdC68); these vectors can be derived from, for example, human, chimpanzee, or rhesus adenoviruses. In some embodiments, the adenovirus is Ad26. Examples of adenoviral vectors useful for the delivery of a polynucleotide of interest is described in WO2006040330, the disclosure of which is herein incorporated by reference.

The term “adjuvant” as used herein means any molecule added to the vaccine described herein to enhance the immunogenicity of the antigen.

As used herein, by “administering” is meant a method of giving a dosage of a pharmaceutical composition (e.g., an immunogenic composition such as a vaccine composition, preferably, a SARS-CoV-2 vaccine) to a subject. The compositions utilized in the methods described herein can be administered, for example, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, by gavage, in cremes, or in lipid compositions. The preferred method of administration can vary depending on various factors (e.g., the components of the composition being administered and the severity of the condition being treated).

The term “antibody” as and “immunoglobulin (lg)” are used interchangeably in the broadest sense and include monoclonal antibodies (e.g., full-length or intact monoclonal antibodies), polyclonal antibodies, multivalent antibodies, multispecific antibodies (e.g., bispecific antibodies so long as they exhibit the desired biological activity) and may also include certain antibody fragments. An antibody typically comprises both “light chains” and “heavy chains.” The light chains of antibodies (immunoglobulins) from any vertebrate species can be assigned to one of two clearly distinct types, called kappa (K) and lambda (l), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., lgG1, lgG2, lgG3, 1gG4, IgM, lA1, lgA2, sIgA, IgD or IgE. The heavy chain constant domains that correspond to the different classes of immunoglobulins are called a, d, e, g, and m, respectively. The subunit structures and three-dimensional configurations of different classes of immunoglobulins are well known. The term “codon” as used herein refers to any group of three consecutive nucleotide bases in a given messenger RNA molecule, or coding strand DNA, that specifies a particular amino acid or a starting or stopping signal for translation. The term codon also refers to base triplets in a DNA strand. The fragments of immunoglobulin molecules, such as Fab, Fab′, (Fab′)₂, Fv, and single chain antibody (SCA or scFv) fragments, that are capable of binding to an epitope of an antigen. These antibody fragments, which retain some ability to selectively bind to an antigen (e.g., a polypeptide antigen) of the antibody from which they are derived, can be made using well known methods in the art (see, e.g., Harlow and Lane, supra), and are described further, as follows. Antibodies can be used to isolate preparative quantities of the antigen by immunoaffinity chromatography. Chimeric, human-like, humanized or fully human antibodies are particularly useful for administration to human patients. The antibody can be an antibody isolated from the serum sample of a mammal, a polyclonal antibody, affinity purified antibody, or mixtures thereof which exhibit sufficient binding specificity to a desired epitope or a sequence derived therefrom.

A “nanobody” is a single-chain monoclonal antibody, derived from the variable domain (V_{H}H) of variant heavy chain-only IgGs (HCAb) found in camelids (e.g., llamas, alpacas, and camels). They can bind in modes different from typical antibodies, covering more chemical space and binding with very high affinities (Jovcevska and Muyldermans, BioDrugs, 34, 10.1007/s40259-019-00392-z, (2019); Muyldermans S., Ann. Rev. Biochem., 82: 775-797 (2013), the disclosures of which are incorporated herein by reference in their entirety). Nanobodies have a small size, are highly soluble, and are readily cloned and produced in bacteria or yeast (Muyldermans, 2013). They have low immunogenicity (Jovcevska and Muyldermans, 2019), can be ‘humanized’ (Vincke et al., J. Biol. Chem., 284: 3273-3284 (2009)), and modified to improve characteristics (Chanier and Chames, Antibodies, 8: E13 (2019); the disclosures of which are incorporated herein by reference in their entirety).

An Fab fragment consists of a monovalent antigen-binding fragment of an antibody molecule, and can be produced by digestion of a whole antibody molecule with the enzyme papain, to yield a fragment consisting of an intact light chain and a portion of a heavy chain.

An Fab′ fragment of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner.

An (Fab′)2 fragment of an antibody can be obtained by treating a whole antibody molecule with the enzyme pepsin, without subsequent reduction. A (Fab′)₂ fragment is a dimer of two Fab′ fragments, held together by two disulfide bonds.

An Fv fragment is defined as a genetically engineered fragment containing the variable region of a light chain and the variable region of a heavy chain expressed as two chains.

A single chain antibody (“SCA” or scFv) is a genetically engineered single chain molecule containing the variable region of a light chain and the variable region of a heavy chain, linked by a suitable, flexible polypeptide liner, and which may include additional amino acid sequences at the amino- and/or carboxyl-termini. For example, a single chain antibody may include a tether segment for linking to the encoding polynucleotide. A functional single chain antibody generally contains a sufficient portion of the variable region of a light chain and a sufficient region of the variable region of a heavy chain so as to retain the property of a full-length antibody for binding to a specific target molecule or epitope.

The term “epitope” or “antigenic determinant” as used herein refers to a site on an antigen to which an antibody binds. Epitopes can be formed both from contiguous amino acids (linear epitope) or noncontiguous amino acids juxtaposed by tertiary folding of a protein (conformational epitopes). Epitopes formed from contiguous amino acids are typically retained on exposure to denaturing solvents whereas epitopes formed by tertiary folding are typically lost on treatment with denaturing solvents. An epitope can comprise 3 or more amino acids. Usually an epitope consists of at least 5 to 7 amino acids (such as 5, 6, or 7 amino acids in an epitope), or of at least 8-11 amino acids (such as 8, 9, 10 or 11 amino acids in an epitope), or of more than 11 amino acids (such as 12, 13, 14, 15, 16, 17, 18, 19 or 20 amino acid in an epitope), or of more than 20 amino acids (such as 21, 22, 23, 24, 25, 26, 27, 28, 29, or 30 amino acid in an epitope), less frequently even of 31-40 amino acids. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996), the disclosure of which is incorporated herein by reference in its entirety. A preferred method for epitope mapping on an antigen is surface plasmon resonance.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art that have been described for expression in a variety of eukaryotic and prokaryotic hosts, and may be used for gene therapy as well as for simple protein expression.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of techniques such as, for example, vector-mediated gene transfer (e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are capable of mediating transfer of genes to mammalian cells.

By “gene product” is meant to include mRNAs or other nucleic acids (e.g., microRNAs) transcribed from a gene, as well as polypeptides translated from those mRNAs. In some embodiments, the gene product is from a virus (e.g., a SARS-CoV-2) and may include, for example, any one or more of the viral proteins, or fragments thereof, described herein.

By “heterologous nucleic acid molecule” is meant a nucleotide sequence that may encode proteins derived or obtained from pathogenic organisms, such as viruses, which may be incorporated into a polynucleotide or vector. Heterologous nucleic acids may also encode synthetic or artificial proteins, such as immunogenic epitopes, constructed to induce immunity. An example of a heterologous nucleic acid molecule is one that encodes one or more immunogenic peptides or polypeptides derived from a SARS-CoV-2. The heterologous nucleic acid molecule is one that is not normally associated with the other nucleic acid molecules found in the polynucleotide or vector into which the heterologous nucleic acid molecule is incorporated.

The term “host cell,” refers to cells into which an exogenous nucleic acid has been introduced, including the progeny of such cells. Host cells include “transformants” and “transformed cells,” which include the primary transformed cell and progeny derived therefrom without regard to the number of passages. Host cells include cells within the body of a subject (e.g., a mammalian subject (e.g., a human)) into which an exogenous nucleic acid has been introduced.

By “immunogen” is meant any polypeptide that can induce an immune response in a subject upon administration. In some embodiments, the immunogen is encoded by a nucleic acid molecule that may be incorporated into, for example, a polynucleotide or vector, for subsequent expression of the immunogen (e.g., a gene product of interest, or fragment thereof (e.g., a polypeptide)).

The term “immunogenic composition” as used herein, is defined as material used to provoke an immune response and may confer immunity after administration of the immunogenic composition to a subject.

An “individual,” “patient” or “subject” is a human or an animal. For example, the subject is a mammal selected from domesticated animals (e.g., cows, sheep, cats, dogs, and horses), primates (e.g., humans and non-human primates such as monkeys), rabbits, and rodents (e.g., mice and rats).

By “isolated” is meant separated, recovered, or purified from a component of its natural environment. For example, a nucleic acid molecule or polypeptide may be isolated from a component of its natural environment by 1% (2%, 3%, 4%, 5%, 6%, 7%, 8% 9% 10%, 20%, 30%, 40%, 50%, 60% 70%, 80%, or 90%) or more.

The terms “linked” or “links” or “link” as used herein are meant to refer to the covalent joining of two amino acid sequences or two nucleic acid sequences together through peptide or phosphodiester bonds, respectively, such joining can include any number of additional amino acid or nucleic acid sequences between the two amino acid sequences or nucleic acid sequences that are being joined.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “package insert” as used herein is used to refer to instructions customarily included in commercial packages of therapeutic products, that contain information about the indications, usage, dosage, administration, combination therapy, contraindications and/or warnings concerning the use of such therapeutic products.

“Nucleic acid molecule” or “polynucleotide,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label.

A “nucleic acid vaccine” refers to a vaccine that includes a heterologous nucleic acid molecule under the control of a promoter for expression in a subject. The heterologous nucleic acid molecule can be incorporated into an expression vector, such as a plasmid. A “DNA vaccine” refers to a vaccine in which the nucleic acid is DNA. An “RNA vaccine” refers to a vaccine in which the nucleic acid is RNA (e.g., an mRNA).

A nucleic acid is “operably linked” when it is placed into a structural or functional relationship with another nucleic acid sequence. For example, one segment of DNA may be operably linked to another segment of DNA if they are positioned relative to one another on the same contiguous DNA molecule and have a structural or functional relationship, such as a promoter or enhancer that is positioned relative to a coding sequence so as to facilitate transcription of the coding sequence; a ribosome binding site that is positioned relative to a coding sequence so as to facilitate translation; or a pre-sequence or secretory leader that is positioned relative to a coding sequence so as to facilitate expression of a pre-protein (e.g., a pre-protein that participates in the secretion of the encoded polypeptide). In other examples, the operably linked nucleic acid sequences are not contiguous, but are positioned in such a way that they have a functional relationship with each other as nucleic acids or as proteins that are expressed by them. Enhancers, for example, do not have to be contiguous. Linking may be accomplished by ligation at convenient restriction sites or by using synthetic oligonucleotide adaptors or linkers.

The polypeptide or protein chimera provided by the present disclosure is not a naturally-occurring polypeptide or protein. Such polypeptide or protein chimera are generated by combining an amino acid sequence of an E3 ubiquitin ligase or an amino acid sequence of a component of an E3 ubiquitin ligase, with an amino acid sequence for an antibody specific to the SARS-CoV-2 spike antigen, which increases the breadth, intensity, depth, or longevity of an antiviral response generated upon administration of a polypeptide or a protein chimera of the present disclosure, or a composition (e.g., vaccine) of the present disclosure which contains such a polypeptide or a protein chimera, to a subject (e.g., a human). Methods of making chimeric genes and chimeric polypeptides or proteins are technically described in the art, such as at William Strohl, “Chimeric Genes, Proteins,” Brenner's Encyclopedia of Genetics (2013), the disclosure of which is incorporated herein by reference in its entirety.

Thus, the polypeptide or the protein chimera of the present disclosure reduces the severity, incidence, or transmissibility of SARS-CoV-2 in the subject. The polypeptide or protein chimera may include a polypeptide or protein encoded by a “parent” viral gene sequence such as the “parent” viral gene sequence encoding a SARS-CoV-2 spike protein. Alternatively, the polypeptide or protein chimera may include a polypeptide or protein encoded by a viral gene sequence that corresponds to analogous sequences from various strains or quasi-species of a virus. Modifications to the polypeptide or protein include amino acid additions, substitutions, and deletions. For example, the polypeptide may be a Spike polypeptide, which may further include a leader/signal sequence (e.g., a Spike signal sequence) and/or linker or spacer sequences. Once the polynucleotide sequence is generated, the corresponding polypeptide can be produced or administered by standard techniques (e.g., recombinant viral vectors, such as the adenoviral vectors disclosed in International Patent Application Publications WO 2006/040330 and WO 2007/104792, herein incorporated by reference).

The terms “optimized codon” and “codon optimized” as used herein refer to a codon sequence that has been modified to match codon frequencies in a target (e.g., a subject) or host organism, but that does not alter the amino acid sequence of the original translated protein.

By “pharmaceutical composition” is meant any composition that contains a therapeutically or biologically active agent, such as an immunogenic composition or vaccine (e.g., a polypeptide or a protein chimera described herein, and/or a vector comprising a nucleic acid encoding a polypeptide or a protein chimera described herein), that is suitable for administration to a subject and that treats or prevents a SARS-CoV-2 infection or reduces or ameliorates one or more symptoms of the disease (e.g., SARS-CoV-2 viral titer, viral spread, infection, and/or virus assembly). For the purposes of this invention, pharmaceutical compositions include vaccines, and pharmaceutical compositions suitable for delivering a therapeutic or biologically active agent and can include, for example, tablets, gelcaps, capsules, pills, powders, granulates, suspensions, emulsions, solutions, gels, hydrogels, oral gels, pastes, eye drops, ointments, creams, plasters, drenches, delivery devices, suppositories, enemas, injectables, implants, sprays, aerosols, inhalants, or nebulizers. Any of these formulations can be prepared by well-known and accepted methods of art. See, for example, Remington: The Science and Practice of Pharmacy (21^{st} ed.), ed. A. R. Gennaro, Lippincott Williams & Wilkins, 2005, and Encyclopedia of Pharmaceutical Technology, ed. J. Swarbrick, Informa Healthcare, 2006, each of which is hereby incorporated by reference.

The term “pharmaceutical formulation” as used herein refers to a preparation which is in such form as to permit the biological activity of an active ingredient contained therein to be effective, and which contains no additional components which are unacceptably toxic to a subject to which the formulation would be administered.

By “pharmaceutically acceptable diluent, excipient, carrier, or adjuvant” is meant a diluent, excipient, carrier, or adjuvant that is physiologically acceptable to the subject while retaining the therapeutic properties of the pharmaceutical composition with which it is administered. One exemplary pharmaceutically acceptable carrier is physiological saline. Other physiologically acceptable diluents, excipients, carriers, or adjuvants and their formulations are known to one skilled in the art (see, e.g., U.S. Pub. No. 2012/0076812 which is incorporated by reference herein in its entirety).

The terms “purified” and “isolated” used herein refer to an antibody according to the invention or to a nucleotide sequence, that the indicated molecule is present in the substantial absence of other biological macromolecules of the same type. The term “purified” as used herein preferably means at least 75% by weight, more preferably at least 85% by weight, more preferably still at least 95% by weight, and most preferably at least 98% by weight, of biological macromolecules of the same type are present. An “isolated” nucleic acid molecule which encodes a particular polypeptide refers to a nucleic acid molecule which is substantially free of other nucleic acid molecules that do not encode the polypeptide; however, the molecule may include some additional bases or moieties which do not deleteriously affect the basic characteristics of the composition.

The term “polypeptide” as used herein refers to a polymer in which the monomers are amino acids and are joined together through peptide bonds. A polypeptide may be a full-length naturally-occurring amino acid chain or a fragment, mutant or variant thereof, such as a selected region of the amino acid chain that is of interest in a binding interaction. A polypeptide may also be a synthetic amino acid chain, or a combination of a naturally-occurring amino acid chain or fragment thereof and a synthetic amino acid chain. A fragment refers to an amino acid sequence that is a portion of a full-length protein, and will be typically between about 8 and about 500 amino acids in length, about 8 to about 300 amino acids, about 8 to about 200 amino acids, and about 10 to about 50 or 100 amino acids in length. Additionally, amino acids other than naturally-occurring amino acids, for example β-alanine, phenyl glycine and homoarginine, may be included in the polypeptides. Commonly-encountered amino acids which are not gene-encoded may also be included in the polypeptides. The amino acids may be either the D- or L-optical isomer. In addition, other peptidomimetics are also useful, e.g. in linker sequences of polypeptides (see Spatola, 1983, in Chemistry and Biochemistry of Amino Acids, Peptides and Proteins, Weinstein, ed., Marcel Dekker, New York, p. 267). In general, the term “protein” is not intended to convey any significant difference from the term “polypeptide” other than to include structures which may comprise two or several polypeptide chains held together by covalent or non-covalent bonds.

By “portion” or “fragment” is meant a part of a whole. A portion may comprise at least 10%, 20%, 30%, 40%, 50%, 60%, 70%, 80%, 90%, or 95% of the entire length of a polynucleotide or polypeptide sequence region. For polynucleotides, for example, a portion may include at least 5, 6, 7, 8, 9, 10, 20, 30, 40, 50, 60, 70, 80, 90, 100, 200, 300, 400, 500, 600, 700, 800, 900, 1000, 1100, 1200, 1300, 1400, 1500, 1600, 1700, 1800 or more contiguous nucleotides of a reference polynucleotide molecule. For polypeptides, for example, a portion may include at least 5, 6, 7, 8, 9, 10, 11, 12, 13, 14, 15, 16, 17, 18, 19, 20, 25, 50, 75, 90, 100, 125, 150, 175 200, 225, 250, 275, 300, 325, 350, 375, 400, 425, 450, 475, 500, 525, 550, 575, or 600 or more continuous amino acids of a reference polypeptide molecule.

A “promoter” is a nucleic acid sequence enabling the initiation of the transcription of a gene sequence in a messenger RNA, such transcription being initiated with the binding of an RNA polymerase on or nearby the promoter.

The term “preventing” as used herein refers to avert or avoid a condition from occurring. In some embodiments, preventing is directed to ameliorating the damage associated with a condition, such as a condition related to SARS-CoV-2 infection.

The term “recombinant antibody” as used herein refers to an antibody (e.g. a chimeric, humanized, or human antibody or antigen-binding fragment thereof) that is expressed by a recombinant host cell comprising nucleic acid encoding the antibody. Examples of “host cells” for producing recombinant antibodies include, but are not limited to: (1) mammalian cells, for example, Chinese Hamster Ovary (CHO), COS, myeloma cells (including Y0 and NS0 cells), baby hamster kidney (BHK), Hela and Vero cells; (2) insect cells, for example, sf9, sf21 and Tn5; (3) plant cells, for example plants belonging to the genus Nicotiana (e.g. Nicotiana tabacum); (4) yeast cells, for example, those belonging to the genus Saccharomyces (e.g. Saccharomyces cerevisiae) or the genus Aspergillus (e.g. Aspergillus niger); (5) bacterial cells, for example Escherichia. coli cells or Bacillus subtilis cells, etc.

By “sequence identity” or “sequence similarity” is meant that the identity or similarity, respectively, between two or more amino acid sequences, or two or more nucleotide sequences, is expressed in terms of the identity or similarity between the sequences. Sequence identity can be measured in terms of “percentage (%) identity,” in which a higher percentage indicates greater identity shared between the sequences. Sequence similarity can be measured in terms of percentage similarity (which takes into account conservative amino acid substitutions); the higher the percentage, the more similarity shared between the sequences. Homologs or orthologs of nucleic acid or amino acid sequences possess a relatively high degree of sequence identity/similarity when aligned using standard methods. Sequence identity may be measured using sequence analysis software on the default setting (e.g., Sequence Analysis Software Package of the Genetics Computer Group, University of Wisconsin Biotechnology Center, 1710 University Avenue, Madison, Wl 53705). Such software may match similar sequences by assigning degrees of homology to various substitutions, deletions, and other modifications. Sequence identity/similarity can be determined across all or a defined portion of the two or more sequences compared.

By “signal peptide” is meant a short peptide (e.g., 5-30 amino acids in length) at the N-terminus of a polypeptide that directs a polypeptide towards the secretory pathway (e.g., the extracellular space). The signal peptide is typically cleaved during secretion of the polypeptide. The signal sequence may direct the polypeptide to an intracellular compartment or organelle, e.g., the Golgi apparatus. A signal sequence may be identified by homology, or biological activity, to a peptide with the known function of targeting a polypeptide to a particular region of the cell. One of ordinary skill in the art can identify a signal peptide by using readily available software (e.g., Sequence Analysis Software Package of the Genetics Computer Group, University of Wisconsin Biotechnology Center, 1710 University Avenue, Madison, Ws. 53705, BLAST, or PILEUP/PRETTYBOX programs).

The term “small peptide” as used herein is referred to a peptide consisting of at most 25, 24, 23, 22, 21, 20, 19, 18, 17, 16, 15, 14, 13, 12, 11, 10, 9, 8, 7, 6, 5 amino acid residues. The small peptide may be a linear chain of amino acid residues or a branched chain of amino acid residues. In some embodiments, the small peptide may be a cyclic peptide.

As used herein, the phrase “specifically binds” refers to a binding reaction which is determinative of the presence of an antigen in a heterogeneous population of proteins and other biological molecules that is recognized, e.g., by an antibody or antigen-binding fragment thereof, with particularity. A variety of immunoassay formats may be used to select antibodies specifically immunoreactive with a particular protein or carbohydrate. For example, solid-phase ELISA immunoassays are routinely used to select antibodies specifically immunoreactive with a protein or carbohydrate. See, Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of immunoassay formats and conditions that can be used to determine specific immunoreactivity.

A “subject” is a vertebrate, such as a mammal (e.g., a primate and a human, in particular a human with underlying health conditions (e.g., hypertension, diabetes, or cardiovascular disease)). Mammals also include, but are not limited to, farm animals (such as cows), sport animals (e.g., horses), pets (such as cats, and dogs), mice, rats, bats, civets, and raccoon dogs. A subject to be treated according to the methods described herein, e.g., a subject in need of protection from a SARS-CoV-2 infection or having a SARS-CoV-2 infection, may be one who has been diagnosed by a medical practitioner as having such a need or infection. Diagnosis may be performed by any suitable means. A subject in whom the development of an infection is being prevented may or may not have received such a diagnosis. One skilled in the art will understand that a subject to be treated according to the present invention may have been subjected to standard tests or may have been identified, without examination, as one with a suspected infection or at high risk of infection due to the presence of one or more risk factors (e.g., exposure to a SARS-CoV-2). Additionally, humans with underlying health conditions (e.g., hypertension, diabetes, or cardiovascular disease) are identified as subjects at high risk of infection with a coronavirus (e.g., SARS-CoV-2). The methods of treating a human subject with a composition are, therefore, particularly useful in treating, reducing the severity, reducing the incidence, reducing the transmissibility, and/or preventing a SARS-CoV-2 infection in humans with underlying health conditions.

The term “therapeutically effective amount” as used herein means any amount which, as compared to a corresponding subject who has not received such amount, results in, but is not limited to, treating, ameliorating or reducing the severity, ameliorating or reducing the incidence, ameliorating or reducing the transmissibility, and/or preventing a SARS-CoV-2 infection. The term also includes within its scope amounts effective to enhance normal physiological function as well as amounts effective to cause a physiological function in a patient which enhances or aids in the therapeutic effect of a second pharmaceutical agent.

The term “prophylactically effective amount” as used herein means any amount which, as compared to a corresponding subject who has not received such amount, results in, but is not limited to preventing, ameliorating or reducing the incidence, ameliorating or reducing the transmissibility, of a SARS-CoV-2 infection, or a decrease in the rate of advancement of SARS-CoV-2 infection. The term also includes within its scope amounts effective to enhance normal physiological function as well as amounts effective to cause a physiological function in a patient which enhances or aids in the therapeutic or prophylactic effect of a second pharmaceutical agent.

The term “treating” or “treatment” includes reducing the number of symptoms or reducing the severity, duration, frequency, incidence, or transmissibility of SARS-CoV2 infection in a subject. The term treating can also mean delaying the onset or progression of symptoms, reducing progression of severity of symptoms, associated with SARS-CoV-2 infection of a subject, or of a disease or disorder associated with SARS CoV2 infection in a subject, or increasing the longevity of a subject having a SARS CoV2 infection.

The term “vaccine” as used herein, is defined as material used to provoke an immune response and that confers immunity for a period of time after administration of the vaccine to a subject.

A “variant” may be a nucleic acid sequence that is substantially identical over the full length of the full gene sequence or fragment thereof. For example, the nucleic acid sequence may be at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical over the full length of the nucleic acid sequence or fragment thereof. A variant may be an amino acid sequence that is substantially identical over the full length of the full amino acid sequence or fragment thereof. For example, the amino acid sequence may be at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical over the full length of the amino acid sequence or fragment thereof.

The term “percent (%) amino acid sequence identity” with respect to a reference polypeptide sequence as used herein is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

By “vector” is meant a DNA construct that includes one or more polynucleotides, or fragments thereof, such as from a viral species, such as a SARS-CoV-2 species. The vector can be used to infect cells of a subject, which results in the translation of the polynucleotides of the vector into a protein product. One type of vector is a “plasmid,” which refers to a circular double stranded DNA loop into which additional DNA segments may be ligated. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome.

Moreover, certain vectors are capable of directing the expression of genes to which they are operatively linked. Such vectors are referred to herein as “recombinant expression vectors” (or simply, “recombinant vectors”). In general, expression vectors of utility in recombinant DNA techniques are often in the form of plasmids. In the present specification, “plasmid” and “vector” may, at times, be used interchangeably as the plasmid is the most commonly used form of vector. Other vectors include, e.g., viral vectors, such as adenoviral vectors (e.g., an Ad26 vector), in particular, those described herein.

The term “virus,” as used herein, is defined as an infectious agent that is unable to grow or reproduce outside a host cell and that infects mammals (e.g., humans).

A “viral vector” is defined as a recombinantly produced virus or viral; particle that comprises a polynucleotide to be delivered into a host cell. Examples of viral vectors include retroviral vectors, adenovirus vectors, adeno-associated virus vectors (e.g., see PCT publication no. WO 2006/002203), and the like.

In aspects where gene transfer is mediated by a DNA viral vector, such as an adenovirus (Ad (e.g., Ad26)) or adeno-associated virus (AAV), a vector construct refers to the polynucleotide comprising the viral genome or part thereof, and an E3 ubiquitin ligase or a component of an E3 ubiquitin ligase. Ads are a relatively well characterized, homogenous group of viruses, including over 50 serotypes (WO 95/27071). Ads are easy to grow and do not require integration into the host cell genome. Recombinant Ad derived vectors, particularly those that reduce the potential for recombination and generation of wild-type virus, have also been constructed (WO 95/00655 and WO 95/11984). Vectors that contain both a promoter and a cloning site into which a polynucleotide can be operatively linked are known in the art. Such vectors are capable of transcribing RNA in vitro or in vivo. To optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation.

Other features and advantages will be apparent from the following Detailed Description, the drawings, and the claims.

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods and examples disclosed herein are illustrative only and not intended to be limiting.

The ubiquitin-proteasome system (UPS) is the major proteolytic system that controls protein degradation and therefore regulates many cellular processes in eukaryotic cells, such as DNA repair, stress responses and cell proliferation. The UPS consists of specific enzymes that modify protein substrates with ubiquitin, and 26S proteasomes responsible for proteolysis of ubiquitin-tagging substrates. This ubiquitin conjugation to the substrate is carried out by a multistep cascade reaction consisting of the E1, E2, and E3 enzymes. In brief, ubiquitin-activating enzymes (E1s) use energy from ATP hydrolysis to generate thioester bond between the C-terminal of ubiquitin and a Cys residue in the active catalytic site of E1 enzymes. This activated ubiquitin is then transferred to the ubiquitin-conjugating enzymes (E2s), which forms a thioester bond between E2 enzymes and the ubiquitin. Finally, the charged E2 enzymes cooperate with one of hundreds of ubiquitin ligases (E3s) to transfer the activated ubiquitin to a target substrate. A ubiquitin ligase (also called an E3 ubiquitin ligase) is normally composed of a catalytic domain and a target domain. The catalytic domain recruits an E2 ubiquitin-conjugating enzyme that has been loaded with ubiquitin and assists or directly catalyzes the transfer of ubiquitin from the E2 to a protein substrate, and a targeting domain recognizes the protein substrate to be ubiquitylated. In simple and more general terms, the ligase enables movement of ubiquitin from a ubiquitin carrier to the substrate by some mechanism. The ubiquitin, once it reaches its destination, ends up being attached by an isopeptide bond to a lysine residue, which is part of the target protein.

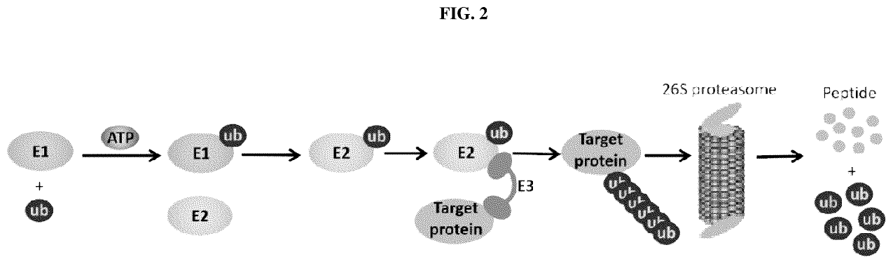

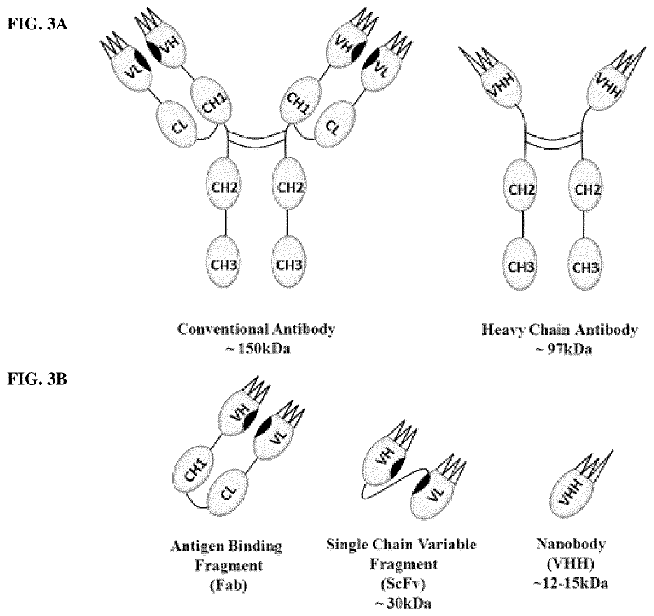

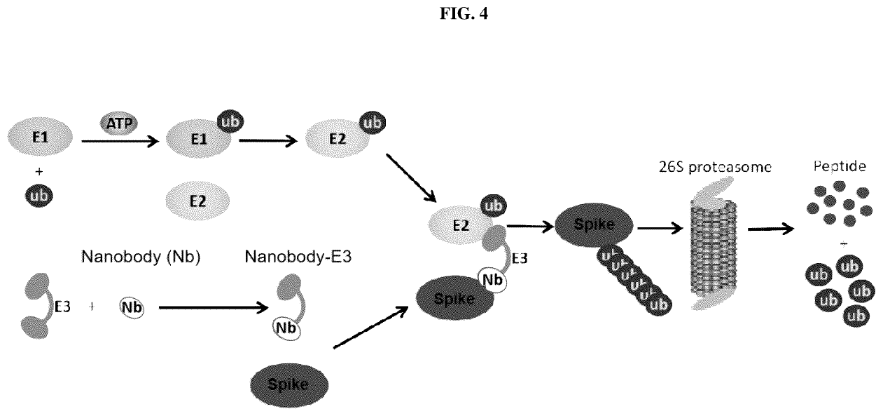

Ubiquitylation involves the sequential transfer of a ubiquitin molecule through an enzyme cascade consisting of a ubiquitin activating enzyme (E1), a ubiquitin conjugating enzyme (E2), and a ubiquitin ligase (E3) until it forms an isopeptide bond between the C terminus of ubiquitin and the ϵ-amino group of a lysine on a substrate protein. Deshaies R. J., Joazeiro C. A., Annu. Rev. Biochem., 78: 399-434 (2009), the disclosure of which is incorporated herein by reference in its entirety. Such a transfer process is shown in FIGS. 2 and 4. The E2-E3 combination governs the specificity of the target protein for modification and the site of attachment to the substrate protein as well as the chain length and type of linkage between the ubiquitin molecules attached. Deshaies R. J., Joazeiro C. A., Annu. Rev. Biochem., 78: 399-434 (2009).

Proteolysis targeting chimeras (PROTACs) are heterobifunctional compounds consisting of protein targeting ligands linked to recruiters of E3 ubiquitin ligases, which induce the proximity of target proteins with E3 ligases to ubiquitinate and degrade specific proteins in cells. Forte et al., ACS Chem. Biol., 18(4): 897-904 (Mar. 20, 2023), the disclosure of which is incorporated herein by reference in its entirety. The PROTACs chemically induce the proximity of E3 ubiquitin ligases with target proteins that do not natively interact with each other to ubiquitinate and degrade specific proteins through the proteasome. Burslem, G. M.; Crews, C. M. Proteolysis-Targeting Chimeras as Therapeutics and Tools for Biological Discovery. Cell, 181: 102-114 (2020); Schreiber, S. L. The Rise of Molecular Glues. Cell, 184: 3-9 (2021); the disclosures of which are incorporated herein by reference in their entirety.

Antibodies are special proteins produced by a type of lymphocytes called B cells in mammals. It is a protein tetramer composed of a pair of heavy chains with the same amino acid sequence and a pair of light chains with the same amino acid sequence. Bacteria, viruses, and other biological macromolecules (referred to as antigens) invade the body to induce B cells to produce antibodies. The antibodies bind to specific sites (epitopes) of these antigens, allowing these antigens to be recognized and swallowed by immune cells, or induce agglutination of precipitation and therefore, lose activity. Antibodies can be produced by manually injecting specific antigens into animals. After separation and purification, they can be used for clinical testing and disease treatment. A nanobody is a class of single-chain monoclonal antibodies, also known as a single-domain antibody or a VHH antibody, is a fragment of an antibody that consists of only one variable domain from a heavy chain. It is derived from a camelid IgG variant that has no light chain. It can bind selectively and strongly to a specific antigen, like a whole antibody, but has a much smaller size (12-15 kDa) and lower immunogenicity.

Nanobodies have a small size, simple structure, high antigen binding affinity, and stability that provides them with an advantage over larger conventional monoclonal antibodies (Jin B-K et al., Int. J. Mol. Sci., 24(6): 5994 (Mar. 22, 2023)), as well as allows them to bind tightly to epitopes that may be obscured by the glycoprotein coat, and minimizes issues of steric hindrance of multiple antibodies binding to adjacent epitopes as observed with larger immunoglobulin G molecules (Corti et al., Cell, 184: 3086-3108 (2021)); the disclosure of which are incorporated herein by reference in their entirety. In addition, nanobodies have low immunogenicity (Jovcevska and Muyldermans, BioDrugs, 34, 10.1007/s40259-019-00392-z, (2019)) and can be readily ‘humanized’ (with an Fc addition) (Vincke C. et al., J. Biol. Chem., 284: 3273-3284 (2009) and modified to improve characteristics (Chanier T. and P. Chames, Antibodies (Basel), 8(1): 13, (2019)). In the case of respiratory viruses like SARS-CoV-2, nanobodies offer a major advantage with their potential for direct delivery by nebulization deep into the lungs. Nambulli et al., Sci. Adv., 7(22): eabh0319 (2021), the disclosure of which is incorporated herein by reference in its entirety.

The spike protein is not only an essential structural protein of SARS-CoV-2 virus but also a functional mediator of the virus entering into a cell. After infecting a living cell, the virus promptly replicates its genome RNA as well as structural proteins to assemble into numerous new viruses, which are then released to infect other healthy cells.

The present invention provides improved polypeptides, nucleic acids, and compositions as well as methods of generating and using the same to protect against or treat viral pathogen infection, in particular, SARS-CoV-2 infection.

The present disclosure is directed to a polypeptide or a protein chimera that uses antibody targeting to direct enzymes of the ubiquitin-proteasome system (UPS) to a target protein antigen of SARS-CoV-2, to result in ubiquitination of the target protein antigen of SARS-CoV2 and subsequent degradation or proteolysis of the target protein antigen of SARS-CoV-2.

As described herein, the polypeptide or the protein chimera of the invention comprise an antibody directed to a Severe Acute Respiratory Syndrome coronavirus 2 (SARS-CoV-2) Spike (S) protein antigen conjugated to a ubiquitin E3 ligase.

The present inventors generated polypeptides or protein chimeras by replacement of the target domain in an E3 ubiquitin ligase with an antibody with specificity for binding to the spike antigen of SARS-CoV-2 virus. Each polypeptide or protein chimera keeps its function of recruiting E2-Ubiquitin but ubiquitylating the spike protein instead of its original target. The ubiquitylated spike protein is consequently degraded through 26S proteasome.

The polypeptides or protein chimeras of the present invention effectively mediate spike protein degradation (or proteolysis) by the UPS system. In doing so, the polypeptides or protein chimeras of the present disclosure can disrupt the virus assembly and spread of the virus, and therefore, may be used to treat the disastrous SARS-CoV-2, prevent SARS-CoV-2 infection of other host cells, reduce the severity of SARS-CoV-2 infection, reduce the incidence of SARS-CoV-2 infection, and/or reduce the transmissibility of a SARS-CoV-2 infection.

This disclosure also relates to nucleic acids molecules encoding the polypeptide or protein chimera of the above embodiments, and those described below.

The invention provides a chimera molecule having the formula:

Ab-L-P,

wherein Ab is an antibody that specifically binds to a Severe Acute Respiratory Syndrome coronavirus 2 (SARS-CoV-2) Spike (S) protein antigen, the Ab is conjugated to L; L is a linker or a bond covalently or non-covalent bound to Ab and to P; and P is a ubiquitin E3 ligase

Ab is an antibody specific for a SARS-CoV-2 spike antigen. Preferably, the antibody is a single chain monoclonal antibody, such as a nanobody specific for a SARS-CoV-2 spike antigen. The nanobody specific for a SARS-CoV-2 spike antigen includes, but is not limited to, Ty1 or H1-H4 (Hanke, L., Vidakovics Perez, L., Sheward, D. J. et al. Nat Commun 11, 4420 (2020), Huo, J., Le Bas, A., Ruza, R. R. et al. Nat. Struct. Mol Biol. 27, 846-854 (2020); the disclosures of which are incorporated herein in their entirety).

Other nanobodies specific for a SARS-CoV2 spike antigen that may be useful as Ab, include but are not limited to those described in Mast F. D. et al., Highly synergistic combinations of nanobodies that target SARS-CoV-2 and are resistant to escape eLife, 10:e73027 (2021), and those described in Nambulli et al., Sci. Adv., 7(22): eabh0319 (2021), the disclosures of which are incorporated herein in their entirety. Additional examples of nanobodies specific for SARS-CoV2 that may be useful as Ab are listed in Table 1.

TABLE 1
(Valenzuela-Nieto et al., Nanobodies: COVID-19 and Future
Perspectives. Front. Drug. Discov. 2:927164 (2022).

| | REFERENCES |
|---|---|
| | (each of the disclosures of which are incorporated |
| NANOBODIES | herein by reference in their entirety) |
| 2NSP23; 2NSP90 | Esposito, G., et al. (2021). MMR-Based Analysis of Nanobodies |
| | to SARS-CoV-2 Nsp9 Reveals a Possible Antiviral Strategy against |
| | COVID-19. Adv. Etiol 5 (12), 2101113 |
| 6ID10 (5; 6; 15; | Gauhar, A., et al. (2021). Single Domain Shark VNAR Antibodies |
| 40; 70; 71; 75; 96; | Neutralize SARS-CoV-2 Infection in Vitro. FASEB J. 35(11). e21970 |
| 99; 113) | |
| A8-G11-Fo | Lu, Q., et al. (2021). Development of Multivalent Nanobodies |
| | Blocking SARS-Co-V-2 infection by Targeting RBD of Spike |
| | Protein. J. Nanobiotechnol 19 (1), 33 |
| aRBD-2-6; | Ma, H., et al. (2021). Potent Neutralization of SARS-CoV-2 by Hetero- |
| aRBD2-7 | Bivalent Alpaoa Nanobodies Targeting the Spike Receptor-Binding |
| | Domain. J. Virol. 95 |
| C6; H3; C1; F2 | Huo, J., et al. (2021). A Potent SARS-CoV-2 Neutralising Nanobody |
| | Shows Therapeutic Efficacy in the Sytan Golden Hamster Model of |
| | COVID-19. Nat. Commun. 12(1), 5489 |
| H11-D4; H11-H4 | Huo, J., et al. (2020). Neutralizing Nanobodies Bind SARS-CoV-2 |
| | Spike RBD and Block Interaction with ACE2. Nat. Struct. Mol. Biol. |
| | 27 (9), 846-854 |
| k-874A | Haga, K., et al. (2021). Nasal Delivery of Single-Domain Antibody |
| | Improves Symptoms of SARS-CoV-2 infection in as Animal Model. |
| | PLoS Pathog. 17 (10), e1009542 |
| KA1; KC1; KC3 | Zupancic, J.M., et al. (2021). Directed Evolution of Potent Neutralizing |
| | Nanobodies against SARS-CoV-2 Using CDR-Swapping Mutagenesis; |
| | Cell Chem. Biol. 28 (9), 1379-1388 |
| MR3; MR17; | Li, T., et al. (2021). A Synthetic Nanobody Targeting RBD Protects |
| SR4; SR3t | Hamsters from SARS-CoV-2 Infection. Nat. Commun. 12 (1), 4635; |
| | Yao, H., et al. (2021). A High-Affinity RBD-Targeting Nanobody |
| | Improves Fusion Partner's Potency against SARS-CoV-2. PLoS |
| | Pathog. 17 (3). et009328 |
| Nanosota-1 | Ye, G., et al. (2021). The Development of Nanosota-1 as Anti- |
| | SARS-CoV-2 Nanobody Drug Candidates. eLife 10 |
| NB1A7; NB1B11 | Shi, Z., et al. (2022). Structural Basis of Nanobodies Neutralizing |
| | SARS-CoV-2 Variants. Structure 30, 707-720 |
| NB1-Nb2-Fc | Chi, X., et al. (2022). An Ultrapotent RBD-Targeted Biparatopic |
| | Nanobody Neutralizes Broad SARS-CoV-2 Variants. Sig |
| | Transduct Target Ther. 7 (1), 44. |
| Nb11-59 | Gal, J., et al. (2021). A Potent Neutralizing Nanobody against |
| | SARS-CoV-2 with Inhaled Delivery Potential MedComm 2 |
| | (1), 101-113 |
| Nb15; Nb56; | Wu, X., et al. (2021). A Potent Bispecific Nanobody Protects hACE2 |
| Nb12; Nb30 | Mice against SARS-CoV-2 Infection via intranasal Administration. |
| | Cell Rep. 37 (3), 109869 |
| Nb6 | Schoof, M., et al. (2020). An Ultrapotent Synthetic Nanobody |
| | Neutralizes SARS-CoV-2 by Stabilizing inactive Spike, Science |
| | 370 (6523), 1473-1479 |
| Nb91-14b3-hFc | Lu, C., et al. (2021). SARS-CoV-2 Exacerbates Proinflammatory |
| | Responses in Myeloid Cells through C-type Leotin Receptors and |
| | Tweety Family Member 2. Immunity 54 (6), 1304-1319. e9 |
| Nbs 89 | Xiang, Y., et al. (2020). Versatile and Multivalent Nanobodies |
| | Efficiently Neutralize SARS-CoV-2 Science 370 (6523). t479- |
| | 1484; Nambuli, S., et al. (2021). Inhaleable Nanobody (PIN-21) |
| | Prevents and Treats SARS-CoV-2 Infections in Syrian Hamsters at |
| | Ultra-low Doses. Sci. Adv. 7 (22).Sun, D., et al. (2021). Potent |
| | Neutralizing Nanobodies Resist Convergent Ciculating Variants |
| | of SARS-CoV-2 by Targeting Diverse and Conserved Epitopes. |
| | Nat. Commun. t2 (1), 4676. |
| NIH-CoVnb-112 | Esparza, T.J., et al. (2020). High Affinity Nanobodies Block |
| | SARS-CoV-2 Spike Receptor Binding Domain Interaction with |
| | Human Angiotensin Converting Enzyme. Sci. Rep. 10 (1), 23370. |
| NM1226; | Wagner, T.R., et al. (2021). NeutrobodyFlex-monitoring SARS- |
| NM1230 | CoV-2 Neutralizing immune Responses Using Nanobodies. |
| | EMBO Rep. 22 (5), e52325. |
| P205; P5F8; | Favorskaya, I.A., et al. (2022). Single-Domain Antibodies Efficiently |
| P2G1 | Neutralize SARS-CoV-2 Variants of Concern. Front. immunol. |
| | 13, 822159 |
| Re6H06; | Güttier, T., et al. (2021). Neutralization of SARS-CoV-2 by Highly |
| Re9B09; | Potent, Hyperthermostable, and Mutation-tolerant Nanobodies. |
| Re5D08; R28 | Embo J. 40 (19), e107985. |
| S1-49; S1-1; S1- | Mast, F.D., et al. (2021). Highly Synergistic Combinations of |
| 23; S1-45; RBD- | Nanobodies that Target SARS-CoV-2 and Are Resistant to Escape. |
| 9; RBD-35; S2-10; | eLife 10, 73027. |
| S2-40 | |
| S14 | Li., T., et al. (2021). A Synthetic Nanobody Targeting RBD Protects |
| | Hamsters from SARS-CoV-2 Infections. Nat. Commun. 12 (1), 4635 |
| saRBD-1 | Weinstein, J.B., et al. (2022). A Potent Alpaca-Derived Nanobody that |
| | Neutralizes SARS-CoV-2 Variants. iScience 25 (3), 103980 |
| Sb14; Sb16; Sb45; | Ahmad, J., et al. (2021). Structures of Synthetic Nanobody-SARS- |
| Sb68 | CoV-2 Receptor-Binding Domain Complexes Reveal District Sites |
| | of Interaction. J. Biol. Chem. 297 (4), 101202 |
| Sb23 | Custodia, T.F., et al. (2020). Selection, Biophysical and Structural |
| | Analysis of Synthetic Nanobodies that Effectively Neutralize SARS- |
| | CoV-2. Nat. Commun. 11 (1), 5588 |
| SP1b4; SPtD9; | Stefan, M.A., et al. (2021). Development of Potent and Effective |
| SP3H4 | Synthetic SARS-CoV-2 Neutralizing Nanobodies. MAbs 13 (1). |
| | 1958663 |
| SR6v15; Nb21; | Chen, F., Liu, Z, and Jiang, F.(2021). Prospects of Neutralizing |
| SR6 | Nanobodies against SARS-CoV-2. Front. Immunol. 12 690742 |
| TB202-1; | Yuan, T.Z., et al. (2022). Rapid Discovery of Diverse Neutralizing |
| TB202-3; | SARS-CoV-2 Antibodies from Large-Scale Synthetic Phage |
| TB202-63 | Libraries. MAbs t4 (1), 2002296 |
| Ty1; Fu2 | Hanke, L., et al. (2020). An Alpaca Nanobody Neutralizes SARS- |
| | CoV-2 by Blocking Receptor Interaction. Bio [symbol] Hanke, L., et al. |
| | (2022). A Bispecific Mononumeric Nanobody Induces Spike |
| | Trimer Dimers and Neutralizes SARS-CoV-2 In Vivo. Nat. |
| | Commun. 13 (1), 155 |
| VHH-E; VHH-U; | Koenig, [symbol] , et al. (2021). Structure-guided Multivalent Nanobodies |
| VHH-V; VHH-W | Block SARS-CoV-2 Infection and Suppress Mutational Escape. |
| | Science 371, 6530 |
| VHH-Fc | Dong, J., et al. (2020). Development of Multi-specific Humanized |
| | Llama Antibodies Blocking SARS-CoV-2/ace2 Interaction with |
| | High Affinity and Avidity. Emerg. microbes Infect. 9 (1), 1034-1036. |
| VHH72 | Schepens, B., et al. (2021). An Infinity-Enhanced, Broadly |
| | Neutralizing Heavy Chain-Only Antibody Protects against SARS- |
| | CoV-2 Infection in Animal Models. Sci. Transl. Med. 13 (621), |
| | eabi7636. |
| W25 | Valenzuela Nieto, G. et al. (2021). Potent Neutralization of Clinical |
| | Isolates of SARS-CoV-2 D614 and G814 Variants by a Mononumeric, |
| | Sub-nanomolar Affinity Nanobody. Sci. Rep. 11 (1), 3319 |
| WNb2; WNb7; | Pynxm, P., et al. (2021). Nanobody Cocktails Potently Neutralize |
| WNb10 | SARS-CoV-2 D614G N501Y Variant and Protect Mice. Proc. Natl. |
| | Acad. Sci. U.S.A. 118(19), 118 |

[symbol]

indicates data missing or illegible when filed

P is an E3 ubiquitin ligase or a component of an E3 ubiquitin ligase. An example is an E2 recruiting domain of an E3 ubiquitin ligase including, but not limited to, VHL (the von Hippel-Lindau gene), CHIP (C-terminus of Hsc70-interacting protein) or Beta-TRC (Beta transducin repeat-containing) ubiquitin E3 ligase (Lim, S. et al., Proceedings of the National Academy of Sciences, 117(11), 5791-5800 (2020); the disclosure of which is incorporated herein in its entirety).

Additional human E3 ubiquitin ligases useful in the present invention are listed in Table 2 below.

TABLE 2
Human E3 Ubiquitin Ligases

| Gene | | | | Swiss- |
|---|---|---|---|---|
| Symbol | Protein Name | Domain | Ref Seq | Protein |
| AFF4 | AF4/FMR2 family member 4 | UBOX | 7656879 | Q9UHB7 |
| | autocrine motility factor receptor, E3 | | | |
| AMFR | ubiquitin protein ligase | RING | 21071001 | Q9UKV5 |
| | anaphase-promoting complex subunit | | | |
| ANAPC11 | 11 isoform 2 | RING | 574957206 | Q9NYG5 |
| | ankyrin repeat and IBR domain- | | | |
| ANKIB1 | containing protein 1 | RING | 148596953 | Q9P2G1 |
| | apoptosis resistant E3 ubiquitin protein | | | |
| AREL1 | ligase 1 | HECTc | 87116668 | O15033 |
| | ariadne RBR E3 ubiquitin protein ligase | | | |
| ARIH1 | 1 | RING | 187761373 | Q9Y4X5 |
| | ariadne RBR E3 ubiquitin protein ligase | | | |
| ARIH2 | 2 | RING | 5453557 | O95376 |
| BARD1 | BRCA1 associated RING domain 1 | RING | 543583789 | Q99728 |
| BFAR | bifunctional apoptosis regulator | RING | 7706091 | Q9NZS9 |
| BIRC2 | baculoviral IAP repeat containing 2 | RING | 390608639 | Q13490 |
| BIRC3 | baculoviral IAP repeat containing 3 | RING | 33946285 | Q13489 |
| BIRC7 | baculoviral IAP repeat containing 7 | RING | 21536421 | Q96CA5 |
| | baculoviral IAP repeat-containing | | | |
| BIRC8 | protein 8 | RING | 44680139 | Q96P09 |
| | BMI1 polycomb ring finger proto- | | | |
| BMI1 | oncogene | RING | 27883842 | P36226 |
| BRAP | BRCA1 associated protein | RING | 188497705 | Q72569 |
| BRCA1 | breast cancer 1, early onset* | RING | 237681125 | P38398 |
| | Cbl proto-oncogene, E3 ubiquitin protein | | | |
| CBL | ligase | RING | 52426745 | P22681 |
| | Cbl proto-oncogene B, E3 ubiquitin | | | |
| CBLB | protein ligase | RING | 54112420 | Q13191 |
| | Cbl proto-oncogene C, E3 ubiquitin | | | |
| CBLC | protein ligase | RING | 195927030 | Q9ULV8 |
| | Cbl proto-oncogene-like 1, E3 ubiquitin | | | |
| CBLL1 | protein ligase | RING | 546230945 | Q75N03 |
| CCDC36 | coiled-coil domain-containing protein 36 | RING | 206597549 | QBIYA8 |
| | cyclin B1 interacting protein 1, E3 | | | |
| CCNB1IP1 | ubiquitin protein ligase | RING | 116812640 | Q9NPC3 |
| | cell growth regulator with RING finger | | | |
| CGRRF1 | domain protein 1 | RING | 5729765 | Q99675 |
| | checkpoint with forkhead and ring finger | | | |
| CHFR | domains, E3 ubiquitin protein ligase | RING | 239048909 | Q96EP1 |
| | CCR4-NOT transcription complex, | | | |
| CNOT4 | subunit 4 | RING | 300069017 | O95628 |
| CUL9 | cullin-9 | RING | 24307991 | Q8IWT3 |
| | cysteine and histidine-rich protein 1 | | | |
| CYHR1 | isoform 2 precursor | RING | 193788637 | Q6ZMK1 |
| | DC-STAMP domain-containing protein 1 | | | |
| DCST1 | isoform 2 | RING | 219521926 | Q5T197 |
| DTX1 | deltex 1, E3 ubiquitin ligase | RING | 41352718 | Q86Y01 |
| DTX2 | deltex 2, E3 ubiquitin ligase | RING | 156564386 | Q86UW9 |
| DTX3 | deltex 3, E3 ubiquitin ligase | RING | 555943736 | QBN9I9 |
| DTX3L | deltex 3 like, E3 ubiquitin ligase | RING | 19923717 | Q8TDB6 |
| DTX4 | deltex 4, E3 ubiquitin ligase | RING | 663071089 | Q9Y2E6 |
| DZIP3 | DAZ interacting zinc finger protein 3 | RING | 7662244 | Q86Y13 |
| E4F1 | E4F transcription factor 1 | zf-C2H2 | 571026678 | Q66K89 |
| | Fanconi anemia, complementation | | | |
| FANCL | group L | RING | 167860138 | Q9NW38 |
| | G2/M-phase specific E3 ubiquitin | | | |
| G2E3 | protein ligase | HECTc | 809279653 | Q7L622 |
| | HECT domain and ankyrin repeat | | | |
| HACE1 | containing E3 ubiquitin protein ligase 1 | HECTc | 116875852 | Q8IYU2 |
| | HECT domain containing E3 ubiquitin | | | |
| HECTD1 | protein ligase 1 | HECTc | 612339310 | Q9ULT8 |
| | HECT domain containing E3 ubiquitin | | | |
| HECTD2 | protein ligase 2 | HECTc | 546231434 | Q5U5R9 |
| | HECT domain containing E3 ubiquitin | | | |
| HECTD3 | protein ligase 3 | HECT | 157738609 | Q5T447 |
| | HEGT domain containing E3 ubiquitin | | | |
| HECTD4 | protein ligase 4 | HECTc | 359718912 | Q9Y4D8 |
| | HECT, C2 and WW domain containing | | | |
| HECW1 | E3 ubiquitin protein ligase 1 | HECTc | 559098413 | Q76N89 |
| | HECT, C2 and WW domain containing | | | |
| HECW2 | E3 ubiquitin protein ligase 2 | HECTc | 756140743 | Q9P2P5 |
| | HECT and RLD domain containing E3 | | | |
| HERC1 | ubiquitin protein ligase family member 1 | HECTc | 126131099 | Q15751 |
| | HECT and RLD domain containing E3 | | | |
| HERC2 | ubiquitin protein ligase 2* | HECTc | 126032348 | O95714 |
| | HECT and RLD domain containing E3 | | | |
| HERC3 | ubiquitin protein ligase 3 | HECTc | 410110896 | Q15034 |
| | HECT and RLD domain containing E3 | | | |
| HERC4 | ubiquitin protein ligase 4 | HECTc | 499137513 | Q5GLZ8 |
| | HECT and RLD domain containing E3 | | | |
| HERC5 | ubiquitin protein ligase 5 | HECTc | 110825982 | Q9UII4 |
| | HECT and RLD domain containing E3 | | | |
| HERO6 | ubiquitin protein ligase family member 6 | HECTc | 259089405 | Q8IVU3 |
| HLTF | helicase-like transcription factor | RING | 21071054 | Q14527 |
| HUWE1 | HECT, UBA and WWE domain | HECTc | 61676188 | Q7Z6Z7 |
| | containing 1, E3 ubiquitin protein ligase | | | |
| | interferon regulatory factor 2 binding | | | |
| IRF2BP1 | protein 1 | RING | 24308115 | Q8IU81 |
| | interferon regulatory factor 2-binding | | | |
| IRF2BP2 | protein 2 isoform B | RING | 116734706 | Q7Z5L9 |
| | interferon regulatory factor 2-binding | | | |
| IRF2BPL | protein-like | RING | 33457336 | Q9H1B7 |
| Itch | itchy E3 ubiquitin protein ligase | HECTc | 380420338 | Q96J02 |
| KCMF1 | potassium channel modulatory factor 1 | RING | 46852178 | Q9P0J7 |
| KMTZC | histone-lysine N-methyltransferase 2C | RING | 91718902 | Q8NEZ4 |
| KMT2D | histone-lysine N-methyltransferase 2D | RING | 148762969 | O14686 |
| | ligand of numb-protein X 1, E3 ubiquitin | | | |
| LNX1 | protein ligase | RING | 187607085 | Q8TBB1 |
| LNX2 | ligand of numb-protein X 2 | RING | 24025688 | Q8N448 |
| | LON peptidase N-terminal domain and | | | |
| LONRF1 | RING finger protein 1 | RING | 87080813 | Q17RB8 |
| | LON peptidase N-terminal domain and | | | |
| LONRF2 | RING finger protein 2 | RING | 148528975 | Q1L529 |
| | LON peptidase N-terminal domain and | | | |
| LONRF3 | RING finger protein 3 isoform 3 | RING | 574280908 | Q496Y0 |
| | leucine rich repeat and sterile alpha | | | |
| LRSAM1 | motif containing 1 | RING | 299758423 | Q6UWE0 |
| LTN1 | listerin E3 ubiquitin protein ligase 1 | RING | 231573214 | O94822 |
| | macrophage erythroblast attacher | | | |
| MAEA | isoform 4 | RING | 661860329 | Q7L5Y9 |
| | mitogen-activated protein kinase kinase | | | |
| MAP3K1 | kinase 1 | RING | 153945765 | Q13233 |
| | membrane-associated ring finger | | | |
| MARCH1 | (C3HC4) 1, E3 ubiquitin protein ligase | RING | 261823980 | Q8TCQ1 |
| | membrane-associated ring finger | | | |
| MARCH10 | (C3HC4) 10, E3 ubiquitin protein ligase | RING | 571026626 | Q8NA82 |
| | membrane-associated ring finger | | | |
| MARCH11 | (C3HC4) 11 | RING | 156523244 | A6NNE9 |
| | membrane-associated ring finger | | | |
| MARCH2 | (C3HC4) 2, E3 ubiquitin protein ligase | RING | 53729334 | Q9P0N8 |
| | membrane-associated ring finger | | | |
| MARCH3 | (C3HC4) 3, E3 ubiquitin protein ligase | RING | 30425370 | Q86UD3 |
| | membrane-associated ring finger | | | |
| MARCH4 | (C3HC4) 4, E3 ubiquitin protein ligase | RING | 57977325 | Q9P2E8 |
| | membrane-associated ring finger | | | |
| MARCH5 | (C3HC4) 5 | RING | 8923415 | Q9NX47 |
| | membrane-associated ring finger | | | |
| MARCH6 | (C3HC4) 6, E3 ubiquitin protein ligase | RING | 397787564 | O60337 |
| | membrane-associated ring finger | | | |
| MARCH7 | (C3HC4) 7, E3 ubiquitin protein ligase | RING | 544346245 | Q9H992 |
| | membrane-associated ring finger | | | |
| MARCH8 | (C3HC4) 8, E3 ubiquitin protein ligase | RING | 544583444 | Q5T0T0 |
| | membrane-associated ring finger | | | |
| MARCH9 | (C3HC4) 9 | RING | 40255016 | Q86YJ5 |
| | MDM2 oncogene, E3 ubiquitin protein | | | |
| Mdm2 | ligase | RING | 510937018 | Q00987 |
| MDM4 | protein Mdm4 isoform 7 | RING | 512125239 | O15151 |
| | MDS1 and EVI1 complex locus protein | | | |
| MECOM | EVI1 isoform b | RING | 327180722 | Q03112 |
| MEX3A | RNA-binding protein MEX3A | RING | 147902746 | A1L020 |
| MEX3B | RNA-binding protein MEX3B | RING | 47716512 | Q6ZN04 |
| MEX3C | mex-3 RNA binding family member C | RING | 148229134 | Q5U5Q3 |
| MEX3D | RNA-binding protein MEX3D isoform 2 | RING | 291621664 | Q86XN8 |
| | mahogunin ring finger 1, E3 ubiquitin | | | |
| MGRN1 | protein ligase | RING | 334883182 | O60291 |
| MIB1 | mindbomb E3 ubiquitin protein ligase 1 | RING | 30348954 | Q86YT6 |
| MIB2 | mindbomb E3 ubiquitin protein ligase 2 | RING | 282394038 | Q96AX9 |
| MID1 | midline 1 | RING | 300797215 | O15344 |
| MID2 | midline 2 | RING | 223890259 | Q9UJV3 |
| MKRN1 | makorin ring finger protein 1 | RING | 619329024 | Q9UHC7 |
| MKRN2 | makorin ring finger protein 2 | RING | 411147393 | Q9H000 |
| MKRN3 | makorin ring finger protein 3 | RING | 5032243 | Q13064 |
| | Putative E3 ubiquitin-protein ligase | | | |
| MKRN4P | makorin-4 | RING | 17368441 | Q13434 |
| | CDK-activating kinase assembly factor | | | |
| MNAT1 | MAT1 isoform 2 | RING | 295789085 | P51948 |
| | male-specific lethal 2 homolog | | | |
| MSL2 | (Drosophila) | RING | 224028284 | Q9HC17 |
| | mitochondrial E3 ubiquitin protein ligase | | | |
| MUL1 | 1* | RING | 171542821 | Q969V5 |
| | MYC binding protein 2, E3 ubiquitin | | | |
| MYCBP2 | protein ligase | RING | 291190787 | O75592 |
| | myosin regulatory light chain interacting | | | |
| MYLIP | protein | RING | 38788243 | Q8WY64 |
| | neural precursor cell expressed, | | | |
| | developmentally down-regulated 4, E3 | | | |
| NEDD4 | ubiquitin protein ligase | HEGTc | 547235493 | P46934 |
| | neural precursor cell expressed, | | | |
| | developmentally down-regulated 4-like, | | | |
| NEDD4L | E3 ubiquitin protein ligase | HECTc | 345478677 | Q96PU5 |
| NEURL1 | neuralized E3 ubiquitin protein ligase 1 | RING | 223278368 | O76050 |
| NEURL1B | neuralized E3 ubiquitin protein ligase 1B | RING | 815890887 | A8MQ27 |
| NEURL3 | neuralized E3 ubiquitin protein ligase 3 | RING | 550822415 | Q96EH8 |
| | transcriptional repressor NF-X1 isoform | | | |
| NFX1 | 3 | RING | 22212927 | Q12986 |
| NEXL1 | NF-X1-type zinc finger protein NFXL1 | RING | 520261838 | Q6ZNB6 |
| | NHL repeat containing E3 ubiquitin | | | |
| NHLRC1 | protein ligase 1 | RING | 40255283 | Q6VVB1 |
| NOSIP | nitric oxide synthase-interacting protein | UBOX | 401709927 | Q9Y314 |
| | non-SMC element 1 homolog (S. | | | |
| NSMCE1 | cerevisiae)* | RING | 94721324 | Q8WV22 |
| PARK2 | parkin RBR E3 ubiquitin protein ligase | RING | 169790973 | O60260 |
| PCGF1 | polycomb group ring finger 1 | RING | 109240538 | Q9BSM1 |
| PCGF2 | polycomb group ring finger 2* | RING | 6005964 | P35227 |
| PCGF3 | polycomb group RING finger protein 3 | RING | 31742478 | Q3KNV8 |
| PCGFS | polycomb group RING finger protein 5 | RING | 380036033 | Q86SE9 |
| | polycomb group RING finger protein 6 | | | |
| PCGF6 | isoform a | RING | 58761530 | Q9BYE7 |
| PDZRN3 | PDZ domain containing ring finger 3 | RING | 735367623 | Q9UPQ7 |
| PDZRN4 | PDZ domain containing ring finger 4 | RING | 257196172 | Q6ZMN7 |
| PELI1 | pellino E3 ubiquitin protein ligase 1 | PELI | 11037063 | Q96FA3 |
| | pellino E3 ubiquitin protein ligase family | | | |
| PELI2 | member 2 | PELI | 10864063 | Q9HAT8 |
| | pellino E3 ubiquitin protein ligase family | | | |
| PELI3 | member 3 | PELI | 341823689 | Q8N2H9 |
| | peroxisome biogenesis factor 10 | | | |
| PEX10 | isoform 1 | RING | 24797089 | O60683 |
| PEX12 | peroxisome assembly protein 12 | RING | 4505721 | O00623 |
| PEX2 | peroxisome biogenesis factor 2 | RING | 289063469 | P28328 |
| PHF7 | PHD finger protein 7 isoform 2 | RING | 506325002 | Q9BWX1 |
| | PHD and RING finger domain- | | | |
| PHRF1 | containing protein 1 isoform 1 | RING | 557440888 | Q9P1Y6 |
| | praja ring finger 1, E3 ubiquitin protein | | | |
| PJA1 | ligase | RING | 530421802 | Q8NG27 |
| | praja ring finger 2, E3 ubiquitin protein | | | |
| PJA2 | ligase | RING | 157412255 | O43164 |
| PLAG1 | zinc finger protein PLAG1 isoform b | RING | 167857798 | Q6DJT9 |
| PLAGL1 | zinc finger protein PLAGL1 isoform 1 | RING | 573459768 | Q9UM63 |
| PML | promyelocytic leukemia* | RING | 109637788 | P29590 |
| PPIL2 | peptidylprolyl isomerase (cyclophilin)- | | | |
| | like 2 | UBOX | 22547215 | Q13356 |
| PRPF19 | pre-mRNA processing factor 19 | UBOX | 7657381 | Q9UMS4 |
| RAD18 | RAD18 homolog (S. cerevisiae) | RING | 256818821 | Q9NS91 |
| RAG1 | recombination activating gene 1 | RING | 4557841 | P15918 |
| | 43 kDa receptor-associated protein of | | | |
| RAPSN | the synapse isoform 2 | RING | 38045928 | Q13702 |
| RBBP6 | retinoblastoma binding protein 6 | RING | 33620769 | Q7Z6E9 |
| | RanBP-type and C3HC4-type zinc | | | |
| RBCK1 | finger containing 1 | RING | 144953898 | Q9BYM8 |
| RBX1 | ring-box 1, E3 ubiquitin protein ligase** | RING | 7657508 | P62877 |
| RC3H1 | ring finger and CCCH-type domains 1 | RING | 664806085 | Q5TC82 |
| RC3H2 | ring finger and CCCH-type domains 2 | RING | 156105699 | Q9HBD1 |
| | ring finger and CHY zinc finger domain | | | |
| RCHY1 | containing 1, E3 ubiquitin protein ligase | RING | 512749780 | Q96PM5 |
| | ring finger and FYVE-like domain | | | |
| RFPL | containing E3 ubiquitin protein ligase | RING | 62865649 | Q8WZ73 |
| RFPL1 | ret finger protein-like 1 | RING | 149408130 | O75677 |
| RFPL2 | ret finger protein-like 2 isoform 3 | RING | 227116308 | O75678 |
| RFPL3 | ret finger protein-like 3 isoform 1 | RING | 149408135 | O75679 |
| RFPL4A | ret finger protein-like 4A | RING | 222446629 | A6NLU0 |
| RFPL4AL1 | ret finger protein-like 4A-like protein 1 | RING | 475807831 | F8VTS6 |
| RFPL4B | ret finger protein-like 4B | RING | 153791360 | Q6ZWI9 |
| | ring finger and WD repeat domain 2, E3 | | | |
| RFWD2 | ubiquitin protein ligase | RING | 557786192 | Q8NHY2 |
| RFWD3 | ring finger and WD repeat domain 3 | RING | 71143112 | Q6PCD5 |
| RING1 | ring finger protein 1* | RING | 51479192 | Q06587 |
| RLF | zinc finger protein Rlf | RING | 157671949 | Q13129 |
| | ring finger protein, LIM domain | | | |
| RLIM | interacting | RING | 34452686 | Q9NVW2 |
| RMNDSA | protein RMD5 homolog A | RING | 12232469 | Q9H871 |
| RMND58 | protein RMD5 homolog B isoform a | RING | 571031354 | Q96G75 |
| RNF10 | ring finger protein 10 | RING | 34452681 | Q8N5U6 |
| RNF103 | ring finger protein 103 | RING | 312147329 | O00237 |
| RNF11 | ring finger protein 11 | RING | 7857520 | Q9Y3C5 |
| RNF111 | ring finger protein 111* | RING | 395455085 | Q6ZNA4 |
| RNF112 | RING finger protein 112 | RING | 264681488 | Q9ULX5 |
| RNF113A | RING finger protein 113A | RING | 5902158 | O15541 |
| RNF113B | RING finger protein 113B | RING | 30578416 | Q8IZP6 |
| RNF114 | ring finger protein 114 | RING | 8923898 | Q9Y508 |
| RNF115 | ring finger protein 115 | RING | 530363886 | Q9Y4L5 |
| RNF121 | RING finger protein 121 isoform b | RING | 665506028 | Q9H920 |
| RNF122 | RING finger protein 122 | RING | 38045931 | Q9H9V4 |
| RNF123 | ring finger protein 123 | RING | 37588869 | Q5XPI4 |
| | ring finger protein 125, E3 ubiquitin | | | |
| RNF125 | protein ligase | RING | 37595555 | Q96EQ8 |
| RNF126 | ring finger protein 126 | RING | 37622894 | Q9BV68 |
| | ring finger protein 128, E3 ubiquitin | | | |
| RNF128 | protein ligase | RING | 37588873 | Q8TEB7 |
| RNF13 | ring finger protein 13 | RING | 34577087 | O34567 |
| RNF130 | ring finger protein 130 | RING | 526253083 | Q86XS8 |
| RNF133 | ring finger protein 133 | RING | 21040269 | QBWVZ7 |
| RNF135 | ring finger protein 135 | RING | 297139721 | Q8IUD6 |
| | ring finger protein 138, E3 ubiquitin | | | |
| RNF138 | protein ligase | RING | 395455059 | Q8WVD3 |
| RNF139 | ring finger protein 139 | RING | 21314654 | QBWU17 |
| RNF14 | ring finger protein 14 | RING | 318067966 | Q9UBS8 |
| RNF141 | ring finger protein 141 | RING | 21361493 | Q8WVD5 |
| RNF144A | ring finger protein 144A | RING | 38045938 | P50876 |
| RNF144B | ring finger protein 144B | RING | 50284696 | Q7Z419 |
| RNF145 | RING finger protein 145 isoform 5 | RING | 313661397 | Q96MT1 |
| RNF146 | ring finger protein 146 | RING | 338827727 | Q9NTX7 |
| RNF148 | RING finger protein 148 precursor | RING | 37675277 | Q8N7C7 |
| RNF149 | ring finger protein 149 | RING | 284447287 | Q8NC42 |
| RNF150 | RING finger protein 150 precursor | RING | 58331204 | Q9ULK6 |
| RNF151 | ring finger protein 151 | RING | 87241872 | Q2KHN1 |
| RNF152 | ring finger protein 152 | RING | 27734873 | Q8N8N0 |
| RNF157 | RING finger protein 157 | RING | 58743365 | Q96PX1 |
| RNF165 | RING finger protein 165 isoform 1 | RING | 57165361 | Q6ZSG1 |
| RNF166 | RING finger protein 166 isoform 2 | RING | 284795357 | Q96A37 |
| RNF167 | ring finger protein 167 | RING | 14149702 | Q9H6Y7 |
| | ring finger protein 168, E3 ubiquitin | | | |
| RNF168 | protein ligase* | RING | 31377566 | Q8IYW5 |
| RNF169 | ring finger protein 169 | RING | 148839382 | Q8NCN4 |
| RNF17 | RING finger protein 17 isoform 2 | RING | 297139728 | Q9BXT8 |
| RNF170 | ring finger protein 170 | RING | 237858658 | Q96K19 |
| RNF175 | RING finger protein 175 | RING | 27734859 | Q8N4F7 |
| RNF180 | ring finger protein 180 | RING | 165932346 | Q86T96 |
| RNF181 | ring finger protein 181 | RING | 7706039 | Q9P0P0 |
| RNF182 | ring finger protein 182 | RING | 259013545 | Q8N6D2 |
| RNF183 | RING finger protein 183 | RING | 153252110 | Q96D59 |
| RNF185 | ring finger protein 185 | RING | 209529683 | Q96GF1 |
| RNF186 | RING finger protein 186 | RING | 9506663 | Q9NXI6 |
| RNF187 | ring finger protein 187 | RING | 256225838 | Q5TA31 |
| RNF19A | ring finger protein 19A, RBR E3 | RING | 525459427 | Q9NV58 |
| | ubiquitin protein ligase | | | |
| RNF19B | ring finger protein 19B | RING | 664806060 | Q6ZMZ0 |
| RNF2 | ring finger protein 2* | RING | 6005747 | Q99496 |
| | ring finger protein 20, E3 ubiquitin | | | |
| RNF20 | protein ligase | RING | 34878777 | Q5VTR2 |
| RNF207 | RING finger protein 207 | RING | 124487387 | Q6ZRF8 |
| RNF208 | ring finger protein 208 | RING | 119220603 | Q9H0X6 |
| | probable E3 SUMO-protein ligase | | | |
| RNF212 | RNF212 isoform c* | RING | 300863140 | Q495C1 |
| RNF212B | RING finger protein 212B | RING | 536291168 | A8MTL3 |
| RNF213 | ring finger protein 213 | RING | 597709777 | Q63HN8 |
| RNF214 | RING finger protein 214 isoform 2 | RING | 503775288 | Q8ND24 |
| RNF215 | RING finger protein 215 precursor | RING | 63025220 | Q9Y6U7 |
| RNF216 | ring finger protein 216 | RING | 46370057 | Q9NWF9 |
| RNF217 | ring finger protein 217 | RING | 556562062 | Q8TC41 |
| RNF219 | RING finger protein 219 | RING | 88759348 | Q5W081 |
| RNF220 | ring finger protein 220 | RING | 46397375 | Q5VTB9 |
| | PREDICTED: RING finger protein 222 | | | |
| RNF222 | isoform X1 | RING | 767992469 | AGNCQ9 |
| RNF223 | RING finger protein 223 | RING | 327412333 | EZERA6 |
| | PREDICTED: RING finger protein 224 | | | |
| RNF224 | isoform X1 | RING | 767958041 | P0DH78 |
| | PREDICTED: RING finger protein 225 | | | |
| RNF225 | isoform X1 | RING | 578834770 | M0QZC1 |
| RNF24 | RING finger protein 24 isoform 2 | RING | 197245452 | Q9Y225 |
| RNF25 | ring finger protein 25 | RING | 34878787 | Q96BH1 |
| RNF26 | RING finger protein 26 | RING | 14042925 | Q9BY78 |
| RNF31 | ring finger protein 31 | RING | 109150431 | Q96EP0 |
| RNF32 | RING finger protein 32 isoform c | RING | 815729259 | Q9H0A6 |
| | ring finger protein 34, E3 ubiquitin | | | |
| RNF34 | protein ligase | RING | 378925642 | Q969K3 |
| RNF38 | ring finger protein 38 | RING | 37577185 | Q9H0F5 |
| RNF39 | RING finger protein 39 isoform 1 | RING | 297206763 | Q9H235 |
| RNF4 | ring finger protein 4* | RING | 297139779 | P78317 |
| | ring finger protein 40, E3 ubiquitin | | | |
| RNF40 | protein ligase | RING | 557440901 | O75150 |
| | ring finger protein 41, E3 ubiquitin | | | |
| RNF41 | protein ligase | RING | 338827618 | Q9H4P4 |
| RNF43 | ring finger protein 43 | RING | 770075668 | Q68DV7 |
| RNF44 | RING finger protein 44 | RING | 7662486 | Q7L0R7 |
| | ring finger protein 5, E3 ubiquitin | | | |
| | protein | | | |
| RNF5 | ligase | RING | 5902054 | Q99942 |
| RNF6 | ring finger protein (C3H2C3 type) 6 | RING | 34305295 | Q9Y252 |
| RNF7 | ring finger protein 7** | RING | 319004144 | Q9UBF6 |
| | ring finger protein 8, E3 ubiquitin | | | |
| | protein | | | |
| RNF8 | ligase | RING | 34304336 | O76064 |
| | RING finger and transmembrane | | | |
| RNFT1 | domain-containing protein 1 | RING | 109134327 | Q5M7Z0 |
| | RING finger and transmembrane | | | |
| RNFT2 | domain-containing protein 2 isoform 1 | RING | 158186614 | Q96EX2 |
| | RING finger and SPRY domain- | | | |
| RSPRY1 | containing protein 1 isoform 1 precursor | RING | 762205506 | Q96DX4 |
| SCAF11 | protein SCAF11 | RING | 117676384 | Q99590 |
| SH3RF1 | SH3 domain containing ring finger 1 | RING | 51988887 | Q726J0 |
| SH3RF2 | SH3 domain containing ring finger 2 | RING | 222446607 | QBTEC5 |
| | SH3 domain-containing RING finger | | | |
| SH3RF3 | protein 3 precursor | RING | 150010572 | Q8TEJ3 |
| | SNF2 histone linker PHD RING | | | |
| SHPRH | helicase, E3 ubiquitin protein ligase | RING | 289547541 | Q149N8 |
| SIAH1 | siah E3 ubiquitin protein ligase 1 | RING | 63148618 | Q8IUQ4 |
| SIAH2 | siah E3 ubiquitin protein ligase 2 | RING | 31982899 | O43255 |
| | siah E3 ubiquitin protein ligase family | | | |
| SIAH3 | member 3 | RING | 110578665 | Q8IW03 |
| | SMAD specific E3 ubiquitin protein | | | |
| SMURF1 | ligase 1 | HECTc | 63148618 | Q9HCE7 |
| | SMAD specific E3 ubiquitin protein | | | |
| SMURF2 | ligase 2 | HECTc | 12232397 | Q9HAU4 |
| | STIP1 homology and U-box containing | | | |
| STUB1 | protein 1, E3 ubiquitin protein ligase | UBOX | 645912990 | Q9UNE7 |
| SYVN1 | synovial apoptosis inhibitor 1, synoviolin | RING | 27436927 | Q86TM6 |
| | E3 ubiquitin-protein ligase TM129 | | | |
| TMEM129 | isoform a | RING | 187936961 | A0AVI4 |
| | topoisomerase I binding, | | | |
| | arginine/serine-rich, E3 ubiquitin protein | | | |
| Topors | ligase* | RING | 307344673 | Q9NS56 |
| TRAF2 | TNF receptor-associated factor 2 | RING | 22027612 | Q12933 |
| TRAF3 | TNF receptor-associated factor 3 | RING | 313661483 | Q13114 |
| TRAF4 | TNF receptor-associated factor 4 | RING | 22027622 | Q9BUZ4 |
| TRAF5 | TNF receptor-associated factor 5 | RING | 77404348 | O00463 |
| | TNF receptor-associated factor 6, E3 | | | |
| TRAF6 | ubiquitin protein ligase | RING | 22027630 | Q9Y4K3 |
| | TNF receptor-associated factor 7, E3 | | | |
| TRAF7 | ubiquitin protein ligase* | RING | 45594312 | Q6Q0C0 |
| TRAIP | TRAF interacting protein | RING | 40807469 | Q9BWF2 |
| TRIM10 | tripartite motif-containing protein 10 | RING | 157426898 | Q9UDY6 |
| | isoform 1 | | | |
| TRIM11 | tripartite motif containing 11 | RING | 21630277 | Q96F44 |
| TRIM13 | tripartite motif containing 13 | RING | 55953112 | O60858 |
| TRIM15 | tripartite motif-containing protein 15 | RING | 149193331 | Q9C019 |
| TRIM17 | tripartite motif containing 17 | RING | 198442878 | Q9Y577 |
| TRIM2 | tripartite motif containing 2 | RING | 705044345 | Q9C040 |
| TRIM21 | tripartite motif containing 21 | RING | 15208660 | P19474 |
| TRIM22 | tripartite motif containing 22 | RING | 313760629 | Q8IYM9 |
| TRIM23 | tripartite motif containing 23 | RING | 15208643 | P36406 |
| TRIM24 | tripartite motif containing 24 | RING | 47419911 | O15164 |
| TRIM25 | tripartite motif containing 25 | RING | 68160937 | Q14258 |
| TRIM26 | tripartite motif-containing protein 26 | RING | 338753391 | Q12899 |
| TRIM27 | tripartite motif containing 27 | RING | 5730009 | P14373 |
| TRIM28 | tripartite motif containing 28* | RING | 5032179 | Q123263 |
| | tripartite motif-containing protein 3 | | | |
| TRIM3 | isoform 2 | RING | 351721595 | O75382 |
| TRIM31 | tripartite motif containing 31 | RING | 62865604 | Q9BZY9 |
| TRIM32 | tripartite motif containing 32 | RING | 153792582 | Q13049 |
| TRIM33 | tripartite motif containing 33 | RING | 74027251 | Q9UPN9 |
| | tripartite motif-containing protein 34 | | | |
| TRIM34 | isoform 3 | RING | 145275183 | Q9BYJ4 |
| | tripartite motif-containing protein 35 | | | |
| TRIM35 | isoform 2 | RING | 751368110 | Q9UPQ4 |
| TRIM36 | tripartite motif containing 36 | RING | 663429604 | Q9NQ86 |
| TRIM37 | tripartite motif containing 37 | RING | 52487176 | O94972 |
| TRIM38 | tripartite motif containing 38 | RING | 5454014 | O00635 |
| TRIM39 | tripartite motif containing 39 | RING | 25777698 | Q9HCM9 |
| | E3 ubiquitin-protein ligase TRIM4 | | | |
| TRIM4 | isoform alpha | RING | 301601616 | Q9C037 |
| | tripartite motif-containing protein 40 | | | |
| TRIM40 | isoform a** | RING | 557636691 | Q6P9F5 |
| TRIM41 | tripartite motif containing 41 | RING | 42516574 | Q8WV44 |
| TRIM42 | tripartite motif-containing protein 42 | RING | 301500637 | Q8IWZ5 |
| TRIM43 | tripartite motif-containing protein 43 | RING | 20270353 | Q96BQ3 |
| TRIM438 | tripartite motif-containing protein 43B | RING | 256600212 | A6NCK2 |
| | tripartite motif-containing protein 45 | | | |
| TRIM45 | isoform 2 | RING | 224548923 | Q9H8W5 |
| | tripartite motif-containing protein 46 | | | |
| TRIM46 | isoform 6 | RING | 538260579 | Q7Z4K8 |
| TRIM47 | tripartite motif-containing protein 47 | RING | 54792146 | Q96LD4 |
| TRIM48 | tripartite motif-containing protein 48 | RING | 201862060 | Q8IWZ4 |
| TRIM49 | tripartite motif-containing protein 49 | RING | 9966829 | P0CI25 |
| TRIM49B | tripartite motif-containing protein | RING | 331028811 | A6NDI0 |
| | PREDICTED: tripartite motif-containing | | | |
| TRIM49C | protein 49C isoform X1 | RING | 767970497 | P0CI26 |
| | PREDICTED: tripartite motif-containing | | | |
| TRIM49D1 | protein 49D1 isoform X1 | RING | 767970137 | C9J1S8 |
| TRIM5 | tripartite motif containing 5 | RING | 283046698 | Q9C035 |
| TRIM50 | tripartite motif containing 50 | RING | 527122124 | QB6XT4 |
| TRIM51 | tripartite motif-containing protein 51 | RING | 209862805 | Q9BSJ1 |
| TRIM52 | tripartite motif-containing protein 52 | RING | 14249412 | Q96A61 |
| | tripartite motif-containing protein 54 | | | |
| TRIM54 | isoform 1 | RING | 78482627 | Q9BYV2 |
| | tripartite motif-containing protein 55 | | | |
| TRIM55 | isoform 4 | RING | 34878852 | Q9BYV6 |
| TRIM56 | tripartite motif containing 56 | RING | 30794216 | Q9BRZ2 |
| TRIM58 | E3 ubiquitin-protein ligase TRIM58 | RING | 112421127 | Q8NG06 |
| TRIM59 | tripartite motif containing 59 | RING | 27436877 | QBIWR1 |
| | tripartite motif-containing protein 6 | | | |
| TRIM6 | isoform 3 | RING | 310772223 | Q9C030 |
| TRIM60 | tripartite motif-containing protein 60 | RING | 384475510 | Q495X7 |
| | putative tripartite motif-containing | | | |
| TRIM61 | protein 61 | RING | 60099474 | Q5EBN2 |
| TRIM62 | tripartite motif containing 62 | RING | 217035095 | Q9BVG3 |
| | tripartite motif containing 63, E3 | | | |
| TRIM63 | ubiquitin protein ligase | RING | 19924163 | Q969Q1 |
| TRIM64 | tripartite motif-containing protein 64 | RING | 210147600 | A6NGJ6 |
| | putative tripartite motif-containing | | | |
| TRIM64B | protein 648 | RING | 256542275 | A6NI03 |
| | putative tripartite motif-containing | | | |
| TRIM64C | protein 640 | RING | 331028829 | AGNLI5 |
| | tripartite motif-containing protein 65 | | | |
| TRIM65 | isoform 2 | RING | 371122551 | Q6PJ69 |
| TRIM67 | tripartite motif containing 67 | RING | 134288906 | Q6ZTA4 |
| TRIM68 | tripartite motif containing 68 | RING | 751557653 | Q6AZZ1 |
| TRIM69 | tripartite motif containing 69 | RING | 667478204 | Q86WT6 |
| | tripartite motif-containing protein 7 | | | |
| TRIM7 | isoform 2 | RING | 44680130 | Q9C029 |
| | tripartite motif containing 71, E3 | | | |
| TRIM71 | ubiquitin protein ligase | RING | 84993742 | Q2Q1W2 |
| TRIM72 | tripartite motif-containing protein 72 | RING | 270265876 | Q6ZMU5 |
| TRIM73 | tripartite motif-containing protein 73 | RING | 65285121 | Q86UV7 |
| TRIM74 | tripartite motif-containing protein 74 | RING | 38524612 | Q86UV6 |
| | Putative tripartite motif-containing | | | |
| TRIM75P | protein 75 | RING | 187663991 | A6NK02 |
| | tripartite motif-containing protein 77 | | | |
| TRIM77 | isoform 2 | RING | 429836863 | I1YAP6 |
| TRIM8 | tripartite motif containing 8 | RING | 148596968 | Q9BZR9 |
| TRIM9 | tripartite motif containing 9 | RING | 190341104 | Q9C026 |
| TRIML1 | tripartite motif family-like 1 | RING | 31542779 | Q8N9V2 |
| | probable E3 ubiquitin-protein ligase | | | |
| TRIML2 | TRIML2 isoform 2 | SPRY | 742068547 | Q8N7C3 |
| TRIP12 | thyroid hormone receptor interactor 12 | HECTc | 545746335 | Q14669 |
| TTC3 | tetratricopeptide repeat domain 3 | RING | 49640011 | P53804 |
| UBE3A | ubiquitin protein ligase E3A | HECTc | 19718766 | Q05086 |
| UBE3B | ubiquitin protein ligase E3B | HECTc | 394025737 | Q7Z3V4 |
| UBE3C | ubiquitin protein ligase E3C | HECTc | 187960100 | Q15386 |
| UBE3D | ubiquitin protein ligase E3D | HECT_2 | 750321479 | Q7Z6J8 |
| UBE4A | ubiquitination factor E4A | RING | 323362983 | Q14139 |
| UBE4B | ubiquitination factor E48 | RING | 157739864 | O95155 |
| UBOX5 | U-box domain containing 5 | RING | 389886556 | O94941 |
| | ubiquitin protein ligase E3 component n- | | | |
| UBR1 | recognin 1 | UBR | 28372497 | Q8IWV7 |
| | ubiquitin protein ligase E3 component n- | | | |
| UBR2 | recognin 2 | UBR | 296317280 | Q8IWV8 |
| | ubiquitin protein ligase E3 component n- | | | |
| UBR3 | recognin 3 (putative) | UBR | 160948610 | Q6ZT12 |
| | ubiquitin protein ligase E3 component n- | | | |
| UBR4 | recognin 4 | UBR | 82659109 | Q5T4S7 |
| | ubiquitin protein ligase E3 component n- | | | |
| UBR5 | recognin 5 | HECTc | 544583486 | O95071 |
| | ubiquitin protein ligase E3 component n- | | | |
| UBR7 | recognin 7 (putative) | UBR | 154426322 | Q8N806 |
| | ubiquitin-like with PHD and ring finger | | | |
| UHRF1 | domains 1 | RING | 586798166 | Q96T88 |
| | ubiquitin-like with PHD and ring finger | | | |
| UHRF2 | domains 2, E3 ubiquitin protein ligase | RING | 23312364 | Q96PU4 |
| UNK | RING finger protein unkempt homolog | RING | 33108525 | Q9C0B0 |
| UNKL | unkempt family zinc finger-like | RING | 448824848 | Q9H9P5 |
| | vacuolar protein sorting-associated | | | |
| VPS11 | protein 11 homolog isoform 2 | RING | 589058161 | Q9H270 |
| | vacuolar protein sorting-associated | | | |
| VPS18 | protein 18 homolog | RING | 17978485 | Q9P253 |
| | vacuolar protein sorting-associated | | | |
| VPS41 | protein 41 homolog isoform 1 | RING | 114199475 | P49754 |
| | vacuolar protein sorting-associated | | | |
| VPS8 | protein 8 homolog isoform a | RING | 57863277 | Q8N3P4 |
| WDR59 | WD repeat-containing protein 59 | RING | 58331266 | Q6PJI9 |
| WDSUB1 | WD repeat, sterile alpha motif and U- | RING | 808175923 | Q8N9V3 |
| | box domain containing 1 | | | |
| WWP1 | WW domain containing E3 ubiquitin | HECTc | 13654239 | Q9H0M0 |
| | protein ligase 1 | | | |
| WWP2 | WW domain containing E3 ubiquitin | HECTc | 394581985 | O00308 |
| | protein ligase 2 | | | |
| XIAP | X-linked inhibitor of apoptosis | RING | 324711009 | P98170 |
| | zinc finger and BTB domain-containing | | | |
| ZBTB12 | protein 12 | RING | 32469509 | Q9Y330 |
| | E3 ubiquitin-protein ligase ZFP91 | | | |
| ZFP91 | isoform 2 | zf-C2H2 | 308081964 | Q96JP5 |
| ZFPL1 | zinc finger protein-like 1 | RING | 33300635 | O95159 |
| ZNF280A | zinc finger protein 280A | HECT | 618857456 | P59817 |
| ZNF341 | zinc finger protein 341 isoform 3 | RING | 544711270 | Q9BYN7 |
| ZNF511 | zinc finger protein 511 | RING | 28274701 | Q8NB15 |
| ZNF521 | zinc finger protein 521 isoform 2 | RING | 815891044 | Q96K83 |
| ZNF598 | zinc finger protein 598 | RING | 409264581 | Q86UK7 |
| ZNF645 | zinc finger protein 645 | RING | 22749189 | Q8N7E2 |
| | zinc and ring finger 1, E3 ubiquitin | | | |
| ZNRF1 | protein ligase | RING | 14150005 | Q8ND25 |
| ZNRF2 | zinc and ring finger 2 | RING | 23821044 | Q8NHG8 |
| ZNRF3 | zinc and ring finger 3 | RING | 332801080 | Q9ULT6 |
| ZNRF4 | zinc and ring finger 4 | RING | 150170725 | Q8WWF5 |
| Zswim2 | zinc finger, SWIM-type containing 2 | RING | 71043932 | Q8NEG5 |
| ZXDC | zinc finger protein ZXDC isoform 2 | RING | 98961138 | Q2QGD7 |

In the above embodiment for the chimera molecule, the Ab is a nanobody that specifically binds to the SARS-CoV-2 spike antigen and P is an E2 recruiting domain of the ubiquitin E3 ligase.

In any one of the embodiments above for the chimera molecule the nanobody specific to Spike (S) protein antigen is selected from the group consisting of Ty1 and H11-H4.

In any one of the embodiments above for the chimera molecule, Ab has an amino acid sequence selected from any one of SEQ ID NOs: 11, 22, 64, 66, 67, or 70, or an amino acid sequence having at least 85% sequence identity to the amino acid sequence of any one of SEQ ID NOs: 11, 22, 64, 66, 67, or 70.

In any one of the embodiments above for the chimera molecule, Ab has an amino acid sequence encoded by a nucleic acid sequence selected from any one of SEQ ID NOs: 4, 17, 56, 63, or 65, or an amino acid sequence encoded by a nucleic acid sequence having at least 85% sequence identity to the nucleic acid sequence of any one of SEQ ID NOs: 4, 17, 56, 63, or 65.

In any one of the embodiments above for the chimera molecule, P has an amino acid sequence selected from any one of SEQ ID NOs: 12, 32 or 69, or an amino acid sequence having at least 85% sequence identity to the amino acid sequence of any one of SEQ ID NOs: 12, 32 or 69.

In any one of the embodiments above for the chimera molecule, P has an amino acid sequence encoded by a nucleic acid sequence selected from any one of 5, 27, or 68, or an amino acid sequence encoded by a nucleic acid sequence having at least 85% sequence identity to the nucleic acid sequence of any one of SEQ ID NOs: 5, 27, or 68.

In any one of the embodiments above for the chimera molecule, Ab has an amino acid sequence selected from any one of SEQ ID NOs: 4, 17, 56, 63, or 65, and P has an amino acid sequence selected from any one of SEQ ID NOs: 12, 32 or 69.

In any one of the embodiments above for the chimera molecule, Ab has an amino acid sequence encoded by a nucleic acid sequence selected from any one of SEQ ID NOs: 4, 17, 56, 63, or 65, and P has an amino acid sequence encoded by a nucleic acid sequence selected from any one of 5, 27, or 68.

In any one of the embodiments above, the chimera molecule, is a polypeptide or a protein chimera.

In any one of the embodiments above for the chimera molecule, the polypeptide or protein chimera has an amino acid sequence selected from any one of SEQ ID NOs: 8, 13, 20, 23, 30, 33, 39, 41, 48, 49, 57, or 58, or an amino acid sequence having at least 85% sequence identity to the amino acid sequence of any one of SEQ ID NOs: 8, 13, 20, 23, 30, 33, 39, 41, 48, 49, 57, or 58.

In any one of the embodiments above for the chimera molecule, the polypeptide or protein chimera has an amino acid sequence encoded by a nucleic acid sequence selected from any one of SEQ ID NOs: 1, 6, 15, 18, 25, 28, 35, 37, 43, 44, 52, or 53, or an amino acid sequence encoded by a nucleic acid sequence having at least 85% sequence identity to the nucleic acid sequence of any one of SEQ ID NOs: 1, 6, 15, 18, 25, 28, 35, 37, 43, 44, 52, or 53.

### Antibody (Ab)

As described herein, the Ab component is an antibody, e.g., a monoclonal antibody (mAB) that expresses the specific protein that is targeted by the antibody. The Ab portion can target a cell that expresses an antigen whereby the antigen specific polypeptide or protein chimera of the present disclosure is delivered intracellularly to the target cell.

In some embodiments, Ab is a monoclonal antibody such as a nanobody (single-chain monoclonal antibody), produced using various techniques known to one skilled in the art. For instance, nanobodies are generally described in Tang, Q. et al., Viruses, 13(11): 2214 (2021), and an example of production of nanobody therapeutics for SARS-CoV-2 is described in Ye G. et al., The Development of a Novel Nanobody Therapeutic for SARS-CoV-2. bioRxiv [Preprint]. 2020 Nov. 17: 2020.11.17.386532. doi: 10.1101/2020.11.17.386532.

In one embodiment, the Ab component is a nanobody specific for a SARS-CoV-2 Spike (S) protein antigen. The SARS-CoV-2 Spike (S) protein antigen includes, but is not limited to, Ty1 or H1-H4 (Hanke, L., Vidakovics Perez, L., Sheward, D. J. et al. Nat Commun 11, 4420 (2020); the disclosure of which is incorporated herein in its entirety).

Nanobodies specific for SARS-CoV-2 Spike (S) protein antigen are exemplified in the Example below.

### Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008); the disclosure of which is incorporated herein in its entirety.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991); the disclosures of which are incorporated herein in their entirety. Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas); the disclosure of which is incorporated herein in its entirety. Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3): 185-91 (2005); the disclosures of which are incorporated herein in their entirety.

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### Library-Derived Antibodies

Antibodies for use in a PAC may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004); the disclosures of which are incorporated herein in their entirety.

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994); the disclosure of which is incorporated herein in its entirety. Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993); the disclosure of which is incorporated herein in its entirety. Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992), the disclosure of which is incorporated herein in its entirety. Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360; the disclosures of which are incorporated herein in their entirety.

### Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)); the disclosures of which are incorporated herein in their entirety. In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling); the disclosures of which are incorporated herein in their entirety.

### Multispecific Antibodies

In certain embodiments, an antibody provided herein is a multispecific antibody, e.g. a bispecific antibody. The term “multispecific antibody” as used herein refers to an antibody comprising an antigen-binding domain that has polyepitopic specificity (i.e., is capable of binding to two, or more, different epitopes on one molecule or is capable of binding to epitopes on two, or more, different molecules).

In some embodiments, multispecific antibodies are monoclonal antibodies that have binding specificities for at least two different antigen binding sites (such as a bispecific antibody). In some embodiments, the first antigen-binding domain and the second antigen-binding domain of the multispecific antibody may bind the two epitopes within one and the same molecule (intramolecular binding). For example, the first antigen-binding domain and the second antigen-binding domain of the multispecific antibody may bind to two different epitopes on the same protein molecule. In certain embodiments, the two different epitopes that a multispecific antibody binds are epitopes that are not normally bound at the same time by one monospecific antibody, such as e.g. a conventional antibody or one immunoglobulin single variable domain. In some embodiments, the first antigen-binding domain and the second antigen-binding domain of the multispecific antibody may bind epitopes located within two distinct molecules (intermolecular binding). For example, the first antigen-binding domain of the multispecific antibody may bind to one epitope on one protein molecule, whereas the second antigen-binding domain of the multispecific antibody may bind to another epitope on a different protein molecule, thereby cross-linking the two molecules.

In some embodiments, the antigen-binding domain of a multispecific antibody (such as a bispecific antibody) comprises two VH/VL units, wherein a first VH/VL unit binds to a first epitope and a second VH/VL unit binds to a second epitope, wherein each VH/VL unit comprises a heavy chain variable domain (VH) and a light chain variable domain (VL). Such multispecific antibodies include, but are not limited to, full length antibodies, antibodies having two or more VL and VH domains, and antibody fragments (such as Fab, Fv, dsFv, scFv, diabodies, bispecific diabodies and triabodies, antibody fragments that have been linked covalently or non-covalently). A VH/VL unit that further comprises at least a portion of a heavy chain variable region and/or at least a portion of a light chain variable region may also be referred to as an “arm” or “hemimer” or “half antibody.” In some embodiments, a hemimer comprises a sufficient portion of a heavy chain variable region to allow intramolecular disulfide bonds to be formed with a second hemimer. In some embodiments, a hemimer comprises a knob mutation or a hole mutation, for example, to allow heterodimerization with a second hemimer or half antibody that comprises a complementary hole mutation or knob mutation. Knob mutations and hole mutations are discussed further below.

In certain embodiments, a multispecific antibody provided herein may be a bispecific antibody. The term “bispecific antibody” as used herein refers to a multispecific antibody comprising an antigen-binding domain that is capable of binding to two different epitopes on one molecule or is capable of binding to epitopes on two different molecules. A bispecific antibody may also be referred to herein as having “dual specificity” or as being “dual specific.” Exemplary bispecific antibodies may bind both protein and any other antigen. In certain embodiments, one of the binding specificities is for protein and the other is for CD3. See, e.g., U.S. Pat. No. 5,821,337. In certain embodiments, bispecific antibodies may bind to two different epitopes of the same protein molecule. In certain embodiments, bispecific antibodies may bind to two different epitopes on two different protein molecules. Bispecific antibodies may also be used to localize cytotoxic agents to cells which express protein. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983), WO 93/08829, and Traunecker et al., EMBO J. 10: 3655 (1991); the disclosures of which are incorporated herein in their entirety). Multispecific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991); the disclosures of which are incorporated herein in their entirety.

### Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458; the disclosures of which are incorporated herein in their entirety. For discussion of Fab and F(ab′) 2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003); the disclosures of which are incorporated herein in their entirety.

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

### Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### Substitution, Insertion, and Deletion Variants

In certain embodiments, antibody or antibody fragment variants having one or more amino acid substitutions are provided. Sites of interest for substitutional mutagenesis include the CDRs and framework regions (FRs). Conservative substitutions are shown in Table 3 under the heading of “conservative substitutions.” More substantial changes are provided in Table 3 under the heading of “exemplary substitutions,” and as further described below in reference to amino acid side chain classes. Amino acid substitutions may be introduced into an antibody or antibody fragment of interest and the products screened for a desired activity, e.g., retained/improved antigen binding, or decreased immunogenicity.

TABLE 3
Amino acid substitutions

| Original | Exemplary | Preferred |
|---|---|---|
| Residue | Substitutions | Substitutions |
| Ala (A) | Val; Leu; Ile | Val |
| Arg (R) | Lys; Gln, Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Len (L) | Norleucine; Ile; Val; Met; Ala; Phe | Ile |
| Lys (K) | Arg; Gln, Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more complementarity determining region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol., vol. 207, pp. 179-196, 2008; the disclosure of which is incorporated herein in its entirety), and/or SDRs (a-CDRs), with the resulting variant V_{H} or V_{L} being tested for binding affinity. Affinity maturation by constructing and reselecting from secondary libraries has been described, e.g., in Hoogenboom et al. in Methods in Molecular Biology, vol. 178, pp. 1-37 (2001); the disclosure of which is incorporated herein in its entirety. In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody or antibody fragment to bind antigen. For example, conservative alterations (e.g., conservative substitutions as provided herein) that do not substantially reduce binding affinity may be made in CDRs. Such alterations may be outside of CDR “hotspots” or SDRs. In certain embodiments of the variant V_{H} and V_{L} sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells, Science, vol. 244, pp. 1081-1085, 1989; the disclosure of which is incorporated herein in its entirety. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody or antibody fragment with antigen is affected. Further substitutions may be introduced at the amino acid locations demonstrating functional sensitivity to the initial substitutions. Alternatively, or additionally, a crystal structure of an antigen-antibody complex to identify contact points between the antibody or antibody fragment and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody include the fusion to the N- or C-terminus of the antibody to an enzyme (e.g. for ADEPT) or a polypeptide which increases the serum half-life of the antibody.

Amino acid sequence modification(s) of the antibodies described herein are contemplated. For example, it may be desirable to improve the binding affinity and/or other biological properties of the antibody. It is known that when a humanized antibody is produced by simply grafting only CDRs in V_{H} and V_{L} of an antibody derived from a non-human animal in FRs of the V_{H} and V_{L} of a human antibody, the antigen binding activity is reduced in comparison with that of the original antibody derived from a non-human animal. It is considered that several amino acid residues of the V_{H} and V_{L} of the non-human antibody, not only in CDRs but also in FRs, are directly or indirectly associated with the antigen binding activity. Hence, substitution of these amino acid residues with different amino acid residues derived from FRs of the V_{H} and V_{L} of the human antibody would reduce of the binding activity. In order to resolve the problem, in antibodies grafted with human CDR, attempts have to be made to identify, among amino acid sequences of the FR of the V_{H} and V_{L} of human antibodies, an amino acid residue which is directly associated with binding to the antibody, or which interacts with an amino acid residue of CDR, or which maintains the three-dimensional structure of the antibody and which is directly associated with binding to the antigen. The reduced antigen binding activity could be increased by replacing the identified amino acids with amino acid residues of the original antibody derived from a non-human animal.

Modifications and changes may be made in the structure of the antibodies of the present invention, and in the DNA sequences encoding them, and still obtain a functional molecule that encodes an antibody with desirable characteristics.

In making the changes in the amino sequences, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

A further object of the present invention also encompasses function-conservative variants of the antibodies of the present invention.

Two amino acid sequences are “substantially homologous” or “substantially similar” when greater than 80%, preferably greater than 85%, preferably greater than 90% of the amino acids are identical, or greater than about 90%, preferably greater than 95%, are similar (functionally identical) over the whole length of the shorter sequence. Preferably, the similar or homologous sequences are identified by alignment using, for example, the GCG (Genetics Computer Group, Program Manual for the GCG Package, Version 7, Madison, Wis.) pileup program, or any of sequence comparison algorithms such as BLAST, FASTA, etc.

For example, certain amino acids may be substituted by other amino acids in a protein structure without appreciable loss of activity. Since the interactive capacity and nature of a protein define the protein's biological functional activity, certain amino acid substitutions can be made in a protein sequence, and, of course, in its DNA encoding sequence, while nevertheless obtaining a protein with like properties. It is thus contemplated that various changes may be made in the sequences of the antibodies or antibody fragments of the invention, or corresponding DNA sequences which encode said antibodies or antibody fragments, without appreciable loss of their biological activity.

It is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine.

Another aspect of the disclosure, nucleic acid molecules encoding the antibody (Ab) component as described in each of the above embodiments is provided.

Nucleic acid molecules encoding nanobodies specific for a SARS-CoV-2) Spike (S) protein antigen are exemplified in the Example below.

### Conjugation

The Ab antibody component of the chimera molecule may be conjugated to P (ubiquitin E3 ligase) component through a covalent conjugation or non-covalent conjugation. Covalent conjugation can either be direct or via a linker. In certain embodiments, direct conjugation is by construction of a fusion protein of the antibody and the E3 ligase components (i.e., by genetic fusion of the two genes encoding the antibody and the E3 ligase and expression as a single protein). In certain embodiments, direct conjugation is by formation of a covalent bond between a reactive group on the antibody and a corresponding reactive group on the E3 ligase components of the polypeptide or protein chimera. In certain embodiments, direct conjugation is by modification (i.e., genetic modification) of the antibody to include a reactive group (as non-limiting examples, a sulfhydryl group or a carboxyl group) that forms a covalent attachment to the E3 ligase under appropriate conditions, or vice versa. For example, an amino acid with a desired reactive group (i.e., a cysteine residue) may be introduced into the antibody to form a disulfide bond formed with the E3 ligase. Methods for covalent conjugation of an agent to the antibodies are known in the art (i.e., photocrosslinking, see, e.g., Zatsepin et al. Russ. Chem. Rev., 74: 77-95 (2005)).

In some embodiments, the antibody and the E3 ubiquitin ligase may be non-covalently linked or conjugated by any non-covalent attachment means, including hydrophobic bonds, ionic bonds, electrostatic interactions, and the like, as will be readily understood by one of ordinary skill in the art.

Linkers useful in the present invention include, but are not limited to, a PROTAC (proteolysis targeting chimera) linker which is a crosslinker that connects two functional motifs of a PROTAC, a target protein binder (such as the antibody or nanobody specific for the SARS-CoV-2 spike antigen) and an E3 ligase recruiter. Most commonly used PROTAC linkers include, but are not limited to, a PEG linker, an Alkyl-Chain linker, and an Alkyl/ether linker.

Conjugation may also be performed using a variety of linkers, such as bifunctional protein coupling agents including but not limited to, N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

Peptide linkers, comprised of from one to twenty amino acids joined by peptide bonds, may also be used. In certain such embodiments, the amino acids are selected from the twenty naturally-occurring amino acids. In certain other such embodiments, one or more of the amino acids are selected from glycine, alanine, proline, asparagine, glutamine and lysine.

The linker may be a “cleavable linker” facilitating release of the agent upon delivery to the site of action. For example, an acid-labile linker, peptidase-sensitive linker, photolabile linker, dimethyl linker or disulfide-containing linker (Chari et al., Cancer Res., 52:127-131 (1992); U.S. Pat. No. 5,208,020; the disclosures of which are incorporated herein in their entirety) may be used.

Other linkers may include, but are not limited to, glutaraldehyde, a homobifunctional cross-linker, or a heterobifunctional cross-linker. Glutaraldehyde cross-links polypeptides via their amino moieties. Homobifunctional cross-linkers (e.g., a homobifunctional imidoester, a homobifunctional N-hydroxysuccinimidyl (NHS) ester, or a homobifunctional sulfhydryl reactive cross-linker) contain two or more identical reactive moieties and can be used in a reaction procedure in which the cross-linker is added to a solution containing a mixture of the polypeptides to be linked. Homobifunctional NHS ester and imido esters cross-link polypeptides containing amines. In a mild alkaline pH, imido esters react only with primary amines to form imidoamides, and overall charge of the cross-linked polypeptides is not affected. Homobifunctional sulfhydryl reactive cross-linkers include bismaleimidhexane (BMH), 1,5-difluoro-2,4-dinitrobenzene (DFDNB), and 1,4-di-(3′,2′-pyridyldithio) propinoamido butane (DPDPB).

Heterobifunctional cross-linkers have two or more different reactive moieties (e.g., amine reactive moiety and a sulfhydryl-reactive moiety) and may be cross-linked with one of the antibody (such as a nanobody specific for the SARS-CoV-2 spike antigen) and with the E3 ubiquitin ligase via the amine or sulfhydryl reactive moiety, then reacted with the other via the non-reacted moiety. Other multiple heterobifunctional haloacetyl cross-linkers are available, such as pyridyl disulfide cross-linkers. Carbodiimides are a classic example of heterobifunctional cross-linking reagents for coupling carboxyls to amines, which results in an amide bond.

### Composition, Formulation, Kit

The chimera molecule or polypeptide or protein chimera as described herein above in the present disclosure may be included in pharmaceutical compositions, medical devices, kits, or articles of manufacture for therapeutic, prophylactic or diagnostic use. Suitable pharmaceutical compositions, medical devices, kits, or articles of manufacture are described in detail in the art, for instance, WO 2016/138071.

A composition comprising a chimera molecule, or a polypeptide or protein chimera as described in any of the embodiments above, having the formula:

Ab-L-P,

wherein Ab is an antibody that specifically binds to a Severe Acute Respiratory Syndrome coronavirus 2 (SARS-CoV-2) Spike (S) protein antigen, the Ab is conjugated to L; L is a linker or a bond covalently or non-covalent bound to Ab and to P; and P is a ubiquitin E3 ligase; and at least one pharmaceutically acceptable excipient.

In some embodiments, the pharmaceutical composition may be in a liquid form, a lyophilized form or a liquid form reconstituted from a lyophilized form. The lyophilized preparation is typically reconstituted with a sterile solution prior to administration. The standard procedure for reconstituting a lyophilized composition is to add a volume of pure water (typically about equivalent to the volume removed during lyophilization). Solutions comprising antibacterial agents may also be used for the production of pharmaceutical compositions for parenteral administration; see also Chen, Drug Dev Ind Pharm, vol. 18, pp. 1311-54, 1994; the disclosure of which is incorporated herein in its entirety.

A pharmaceutically acceptable tonicity agent may be included in the composition to modulate the tonicity of the formulation. Exemplary tonicity agents include sodium chloride, potassium chloride, glycerin and any component from the group of amino acids, sugars as well as combinations thereof. In some embodiments, the aqueous formulation is isotonic, although hypertonic or hypotonic solutions may also be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 nM.

A pharmaceutically acceptable surfactant may be added to the composition to reduce aggregation of the formulated multi-specific antibody and/or minimize the formation of particulates in the formulation and/or reduce adsorption. Exemplary surfactants include polyoxyethylensorbitan fatty acid esters, polyoxyethylene alkyl ethers, alkylphenylpolyoxyethylene ethers (Triton-X™), polyoxyethylene-polyoxypropylene copolymer (Poloxamer, Pluronic™), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Exemplary concentrations of surfactant in the composition may range from about 0.001% to about 1% w/v.

A lyoprotectant may be added to the composition in order to protect the labile active ingredient (e.g. a protein) against destabilizing conditions during the lyophilization process. For example, known lyoprotectants include sugars (including glucose and sucrose), polyols (including mannitol, sorbitol and glycerol), and amino acids (including alanine, glycine and glutamic acid). Lyoprotectants can be included in an amount of about 10 nM to 500 nM.

In some embodiments, the composition, containing one or more of a surfactant, a buffer, a stabilizer, and a tonicity agent, is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative selected from ethanol, benzyl alcohol, phenol, m-cresol, p-chlor-m-cresol, methyl or propyl parabens, benzalkonium chloride, and combinations thereof, may be is included in the formulation, e.g., at concentrations ranging from about 0.001 to about 2% (w/v).

In some embodiments, the polypeptide or protein chimera of the present disclosure may be formulated as aerosol and intranasal compositions. The polypeptide or protein chimera of the present disclosure may be formulated as intranasal formulations including vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the multi-specific antibody by the nasal mucosa.

In some embodiments, the polypeptide or protein chimera of the present disclosure may be formulated in a unit dosage forms for oral administration such as syrups, elixirs, and suspensions may be provided where each dosage unit, for example, teaspoonful, tablespoonful, tablet or vile, contains a predetermined amount of the composition. Similarly, unit dosage forms for injection or intravenous administration may comprise the multi-specific antibody in a composition as a solution in sterile water, normal saline or another pharmaceutically acceptable carrier.

In some embodiments, the polypeptide or protein chimera of the present disclosure may be formulated as an injectable formulation. Typically, injectable compositions are prepared as liquid solutions or suspensions, solid forms suitable for solution in, or suspension in, liquid vehicles prior to injection may also be prepared. The preparation may also be the emulsified polypeptide or protein chimera of the present disclosure encapsulated in liposome vehicles.

In some embodiments, the polypeptide or protein chimera of the present disclosure may be formulated in suppositories, the composition will include traditional binders and carriers such as, polyalkylene glycols, or triglycerides. Such compositions may be formed from mixtures containing the multi-specific antibody in the range of about 0.5% to about 10% (w/w), e.g., about 1% to about 2%.

In some embodiments, the polypeptide or protein chimera of the present disclosure is formulated in a controlled release formulation. Controlled release within the scope of this invention means one of a number of extended release dosage forms. The following types of controlled release may be used for the purposes of the present invention: continuous release, delayed release, gradual release, long-term release, programmed release, prolonged release, proportionate release, protracted release, slow release, spaced release, sustained release, timed release, delayed action, extended action, layered-time action, long acting, prolonged action, repeated action, sustained action, and extended release. Further discussions of these terms and methods for making the same may be found in Lesczek Krowczynski, Extended-Release Dosage Forms, 1987 (CRC Press, Inc.).

Controlled release composition may be prepared using methods known in the art. Examples of controlled-release preparations include semipermeable matrices of solid hydrophobic polymers containing the multi-specific antibody in which the matrices are in the form of shaped articles, e.g. films or microcapsules. Examples of sustained-release matrices include polyesters, copolymers of L-glutamic acid and ethyl-L-glutamate, non-degradable ethylene-vinyl acetate, hydrogels, polylactides, degradable lactic acid-glycolic acid copolymers and poly-D-(−)-3-hydroxybutyric acid. Possible loss of biological activity and possible changes in immunogenicity of the multi-specific antibody comprised in sustained-release formulation may be reduced or prevented by using appropriate additives, by controlling moisture content and by developing specific polymer matrix compositions.

Controlled release technologies include both physical systems and chemical systems. Physical systems include reservoir systems with rate-controlling membranes, such as microencapsulation, macroencapsulation, and membrane systems; reservoir systems without rate-controlling membranes, such as hollow fibers, ultra microporous cellulose triacetate, and porous polymeric substrates and foams; monolithic systems, including those systems physically dissolved in non-porous, polymeric, or elastomeric matrices (e.g., nonerodible, erodible, environmental agent ingression, and degradable), and materials physically dispersed in non-porous, polymeric, or elastomeric matrices (e.g., nonerodible, erodible, environmental agent ingression, and degradable); laminated structures, including reservoir layers chemically similar or dissimilar to outer control layers; and other physical methods, such as osmotic pumps, or adsorption onto ion-exchange resins.

Chemical systems include chemical erosion of polymer matrices (e.g., heterogeneous, or homogeneous erosion), or biological erosion of a polymer matrix (e.g., heterogeneous, or homogeneous). Additional discussion of categories of systems for controlled release may be found in Agis F. Kydonieus, Controlled Release Technologies: Methods, Theory and Applications, 1980 (CRC Press, Inc.); the disclosure of which is incorporated herein in its entirety.

There are a number of controlled release drug formulations for oral administration that may be used to formulate the polypeptide or protein chimera of the present disclosure. These controlled release formulations include osmotic pressure-controlled gastrointestinal delivery systems; hydrodynamic pressure-controlled gastrointestinal delivery systems; membrane permeation-controlled gastrointestinal delivery systems, which include microporous membrane permeation-controlled gastrointestinal delivery devices; gastric fluid-resistant intestine targeted controlled-release gastrointestinal delivery devices; gel diffusion-controlled gastrointestinal delivery systems; and ion-exchange-controlled gastrointestinal delivery systems, which include cationic and anionic drugs. Additional information regarding controlled release drug delivery systems may be found in Yie W. Chien, Novel Drug Delivery Systems, 1992 (Marcel Dekker, Inc.); the disclosure of which is incorporated herein in its entirety.

The chimera molecule, or polypeptide or protein chimera of the present disclosure, as described in any one of the embodiments above, may be administered to a patient/subject using any available method and route suitable for drug delivery, including in vivo and ex vivo methods, as well as systemic and localized routes of administration. Conventional and pharmaceutically acceptable routes of administration include intranasal, intramuscular, intratracheal, subcutaneous, intradermal, topical application, intravenous, intraarterial, rectal, nasal, oral, and other enteral and parenteral routes of administration. Routes of administration may be combined, if desired, or adjusted depending upon the multi-specific antibodies and/or the desired effect. The polypeptide or protein chimera of the present disclosure can be administered in a single dose or in multiple doses. In some embodiments, the polypeptide or protein chimera of the present disclosure is administered orally. In some embodiments, the polypeptide or protein chimera of the present disclosure is administered via an inhalational route. In some embodiments, the polypeptide or protein chimera of the present disclosure is administered intranasally. In some embodiments, the polypeptide or protein chimera of the present disclosure is administered locally. In some embodiments, the polypeptide or protein chimera of the present disclosure is administered intracranially. In some embodiments, the polypeptide or protein chimera of the present disclosure is administered intravenously.

In another aspect, the disclosure provides a method of treating or preventing a viral infection in a subject in need thereof, comprising administering a therapeutically effective amount of the chimera molecule or a polypeptide or protein chimera as described herein above, to the subject in need thereof.

In some embodiments, the disclosure provides a method of treating or preventing a SARS-CoV-2 infection in a subject in need thereof, comprising administering a therapeutically effective amount of the chimera molecule or a polypeptide or protein chimera as described herein above, to the subject in need thereof.

In one embodiment, the disclosure provides a method of reducing the severity of a viral infection, reducing the incidence of a viral infection, and/or reducing the transmissibility of a viral infection, comprising administering a therapeutically effective amount of a chimera molecule or a polypeptide or protein chimera as described herein above, to the subject in need thereof. In some embodiments, the disclosure provides a method of reducing the severity of a SARS-CoV-2 infection, reducing the incidence of a viral SARS-CoV-2 infection, and/or reducing the transmissibility of a SARS-CoV-2 infection, comprising administering a therapeutically effective amount of a chimera molecule or a polypeptide or protein chimera as described herein above, to the subject in need thereof.

The above methods also involve administering a chimera molecule or a polypeptide or protein chimera, as described herein above, to a subject with a viral infection or to a subject susceptible to a viral infection, such as a SARS-CoV-2 infection.

In some embodiments, the chimera molecule or polypeptide or protein chimera as described herein above, is administered as a vaccine in a vaccine composition for SARS-CoV-2.

In one embodiment, a kit or article of manufacture comprising a chimera molecule or a polypeptide or protein chimera as described herein above, is provided with a package insert or label indicating that the chimera molecule or polypeptide or protein chimera can be used to treat or prevent a SARS-CoV-2 infection, to reduce severity a SARS-CoV-2 infection, to reduce incidence a SARS-CoV-2 infection, and/or to reduce transmissibility of a SARS-CoV-2 infection.

The following examples serve to more fully describe the manner of using the above-described disclosure, as well as to set forth the best modes contemplated for carrying out various aspects of the disclosure. It is understood that these examples in no way serve to limit the true scope of this disclosure, but rather are presented for illustrative purposes. All references cited herein are incorporated by reference in their entirety.

### EXAMPLES

### Example 1—Preparation of Chimeras

Six protein chimeras were constructed.

Each of them is composed of two different ligands (L1-L2), one ligand (L2) is E2 recruiting domain of VHL, CHIP, or Beta-TRC ubiquitin E3 ligase (L2), and the other ligand (L1) is spike specific nanobody Ty1 or H1-H4.

The codon-optimized genes encoding the 6 protein chimeras were synthesized from Genewiz and Genscript, and then cloned into a pShuttle-CMV vector (ordered from Aligent) between XhoI and EcoRV sites in frame with the Myc and His tags included in the downstream of the vector at the C-terminus. The same strategy was also used to clone the optimized spike protein gene of SARS-CoV-2 (ordered from InvivoGen) into the pShuttle-CMV vector, but added a FLAG tag sequence at the C-Terminus of spike for expression detection. The above pShuttle-CMV vectors containing insert were transformed into E. coli competent cells containing pAdEasy-1 (ordered from Aligent) plasmids and integrated into pAdEasy-1 to form a new pAdEasy-1 expressing the chimera or spike under CMV promoter. This new pAdEasy-1 was transfected into AD293 cells (ordered from Aligent) derived from human HEK293 cells, which were already transformed by sheared adenovirus type 5 DNA. AD-293 cells, like HEK293 cells, produce the adenovirus E1 gene in trans, allowing the production of infectious virus particles when cells are transfected with E1-deleted adenovirus pAdEasy-1 vector.

### Result

The protein chimeras were tested for their ability to degrade spike protein in cells, such as mouse 3T3D cells infected with Adenovirus expressing SARS-CoV-2 spike protein under a CMV promoter.

The cell cultures were also treated with different protein chimeras. After 48 hours, the cells were harvested and lysed in SDS-PAGE loading buffer. The cell lysates were loaded into SDS-PAGE gel, and a Western blot was employed to detect spike protein expression. Because spike protein was constructed with FLAG tag, the 1:2000 diluted HRP conjugated FLAG antibody (Thermo Scientific) was used to detect spike protein expression.

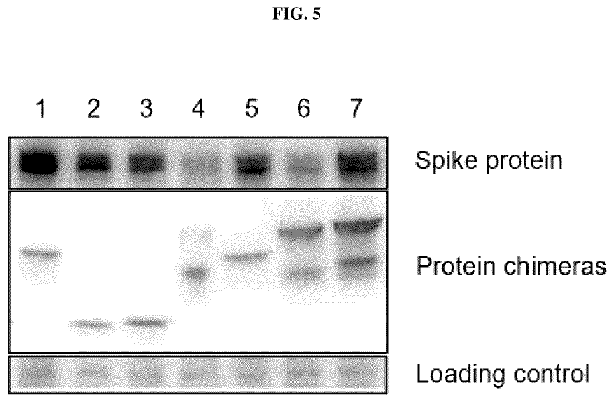

Because spike protein was constructed with FLAG tag, the 1:2000 diluted HRP conjugated FLAG antibody (Thermo Scientific) was used to detect spike protein expression. The result was shown in FIG. 5, which shows Western blot detection of the effect of different protein chimeras on the degradation of spike protein. The middle panel shows protein chimeras expressed in each of the samples. The lower panel was the loading control that indicated the similar levels of total cell lysate loaded into each lane.

Lane 1 was a positive control: a cell lysate that expressed spike protein treated with a nonspecific protein chimera not binding to spike protein as a positive control. Lanes 2 to 7 were spike protein expression cell lysates treated with specific protein chimeras binding to spike protein. Lane 4 and 6 showed very weak spike bands. The spike bands in Lane 3, 4, 5, 7 were slightly weak compared to the positive control Lane 1. The bands of Loading Control were nearly the same in all 7 lanes, indicating that the amount of sample loaded in each lane was the same.

The above result showed that the six protein chimeras, especially OptTy1-CHIP and Beta-Trc-OptTy1, effectively mediated spike protein degradation in cells.

The OptTy1-CHIP and Beta-Trc-OptTy1 were then used to validate their effect in a mouse model.

For each of the sequences below, the bolded portion is the nanobody sequence
and the italics portion is the E3 ligase sequence.
1. Chimera 1 (Lane 2)
OptTy1-VHL DNA sequence (SEQ ID NO: 1)
CAAGTGCAGCTGGTGGAAACCGGCGGCGGACTCGTGCAGCCTGGCGGATCT
CTGCGGCTGAGCTGTGCCGCTTCTGGCTTTACATTCAGCAGCGTGTACATGA
ATTGGGTCAGACAGGCCCCTGGAAAAGGCCCTGAGTGGGTGTCTAGAATCT
CCCCAAACAGCGGCAACATCGGCTACACCGACAGCGTGAAGGGCAGATTCA
CCATCAGCAGAGATAATGCCAAGAACACCCTGTATCTGCAGATGAACAACCT
GAAGCCCGAGGACACCGCCCTGTACTACTGCGCCATCGGCCTGAACCTGAG
CTCCAGCAGCGTGCGGGGCCAGGGCACACAGGTTACAGTGTCCAGCGGATC
CACACTGCCTGTGTACACCCTGAAAGAGCGGTGCCTGCAAGTGGTCAGATCTCTCGTG
AAGCCTGAGAACTACAGAAGACTGGACATCGTGCGGAGCCTGTACGAGGATCTGGAA
GATCACCCCAACGTGCAGAAGGACCTGGAACGGCTGACCCAGGAGAGAATCGCCCAC
CAGAGAATGGGCGAC*
*indicates portion at end of sequence may optionally further include the following sequence
of digestion or protein tag for protein detection (as shown in SEQ ID NO: 2 below):
GGTACCAAGCTTGGGCCCGAACAAAAACTCATCTCAGAAGAGGATCTGAATAGC
GCCGTCGACCATCATCATCATCATCATTGA (SEQ ID NO: 3)
SEQ ID NO: 2:
CAAGTGCAGCTGGTGGAAACCGGCGGCGGACTCGTGCAGCCTGGCGGATCT
CTGCGGCTGAGCTGTGCCGCTTCTGGCTTTACATTCAGCAGCGTGTACATGA
ATTGGGTCAGACAGGCCCCTGGAAAAGGCCCTGAGTGGGTGTCTAGAATCT
CCCCAAACAGCGGCAACATCGGCTACACCGACAGCGTGAAGGGCAGATTCA
CCATCAGCAGAGATAATGCCAAGAACACCCTGTATCTGCAGATGAACAACCT
GAAGCCCGAGGACACCGCCCTGTACTACTGCGCCATCGGCCTGAACCTGAG
CTCCAGCAGCGTGCGGGGCCAGGGCACACAGGTTACAGTGTCCAGCGGATC
CACACTGCCTGTGTACACCCTGAAAGAGCGGTGCCTGCAAGTGGTCAGATCTCTCGTG
AAGCCTGAGAACTACAGAAGACTGGACATCGTGCGGAGCCTGTACGAGGATCTGGAA
GATCACCCCAACGTGCAGAAGGACCTGGAACGGCTGACCCAGGAGAGAATCGCCCAC
CAGAGAATGGGCGACGGTACCAAGCTTGGGCCCGAACAAAAACTCATCTCAGAAG
AGGATCTGAATAGCGCCGTCGACCATCATCATCATCATCATTGA
The sequence bolded above in SEQ ID NO: 1 is SEQ ID NO: 4, and the sequence in
italics above in SEQ ID NO: 1 is SEQ ID NO: 5, as shown below.
SEQ ID NO: 4
**CAAGTGCAGCTGGTGGAAACCGGCGGCGGACTCGTGCAGCCTGGCGGAT
CTCTGCGGCTGAGCTGTGCCGCTTCTGGCTTTACATTCAGCAGCGTGTACAT
GAATTGGGTCAGACAGGCCCCTGGAAAAGGCCCTGAGTGGGTGTCTAGAAT
CTCCCCAAACAGCGGCAACATCGGCTACACCGACAGCGTGAAGGGCAGATT
CACCATCAGCAGAGATAATGCCAAGAACACCCTGTATCTGCAGATGAACAAC
CTGAAGCCCGAGGACACCGCCCTGTACTACTGCGCCATCGGCCTGAACCTG
AGCTCCAGCAGCGTGCGGGGCCAGGGCACACAGGTTACAGTGTCCAGC
**indicates a nucleotide sequence encoding one or more amino acids may be optionally
included. For example, the nucleotide sequence may be ATG (as in SEQ ID NO: 63) or
GGATCC. Such an embodiment includes, for example, SEQ ID NO: 6:
ATGCAAGTGCAGCTGGTGGAAACCGGCGGCGGACTCGTGCAGCCTGGCGG
ATCTCTGCGGCTGAGCTGTGCCGCTTCTGGCTTTACATTCAGCAGCGTGTAC
ATGAATTGGGTCAGACAGGCCCCTGGAAAAGGCCCTGAGTGGGTGTCTAGA
ATCTCCCCAAACAGCGGCAACATCGGCTACACCGACAGCGTGAAGGGCAGA
TTCACCATCAGCAGAGATAATGCCAAGAACACCCTGTATCTGCAGATGAACA
ACCTGAAGCCCGAGGACACCGCCCTGTACTACTGCGCCATCGGCCTGAACC
TGAGCTCCAGCAGCGTGCGGGGCCAGGGCACACAGGTTACAGTGTCCAGCG
GATCCACACTGCCTGTGTACACCCTGAAAGAGCGGTGCCTGCAAGTGGTCAGATCTCT
CGTGAAGCCTGAGAACTACAGAAGACTGGACATCGTGCGGAGCCTGTACGAGGATCT
GGAAGATCACCCCAACGTGCAGAAGGACCTGGAACGGCTGACCCAGGAGAGAATCGC
CCACCAGAGAATGGGCGAC*
*indicates portion at end of sequence may optionally further include the sequence of SEQ ID
NO: 3, as shown in SEQ ID NO: 7 below:
ATGCAAGTGCAGCTGGTGGAAACCGGCGGCGGACTCGTGCAGCCTGGCGG
ATCTCTGCGGCTGAGCTGTGCCGCTTCTGGCTTTACATTCAGCAGCGTGTAC
ATGAATTGGGTCAGACAGGCCCCTGGAAAAGGCCCTGAGTGGGTGTCTAGA
ATCTCCCCAAACAGCGGCAACATCGGCTACACCGACAGCGTGAAGGGCAGA
TTCACCATCAGCAGAGATAATGCCAAGAACACCCTGTATCTGCAGATGAACA
ACCTGAAGCCCGAGGACACCGCCCTGTACTACTGCGCCATCGGCCTGAACC
TGAGCTCCAGCAGCGTGCGGGGCCAGGGCACACAGGTTACAGTGTCCAGCG
GATCCACACTGCCTGTGTACACCCTGAAAGAGCGGTGCCTGCAAGTGGTCAGATCTCT
CGTGAAGCCTGAGAACTACAGAAGACTGGACATCGTGCGGAGCCTGTACGAGGATCT
GGAAGATCACCCCAACGTGCAGAAGGACCTGGAACGGCTGACCCAGGAGAGAATCGC
CCACCAGAGAATGGGCGACGGTACCAAGCTTGGGCCCGAACAAAAACTCATCTCA
GAAGAGGATCTGAATAGCGCCGTCGACCATCATCATCATCATCATTGA
SEQ ID NO: 5
GGATCCACACTGCCTGTGTACACCCTGAAAGAGCGGTGCCTGCAAGTGGTCAGATCTC
TCGTGAAGCCTGAGAACTACAGAAGACTGGACATCGTGCGGAGCCTGTACGAGGATCT
GGAAGATCACCCCAACGTGCAGAAGGACCTGGAACGGCTGACCCAGGAGAGAATCGC
CCACCAGAGAATGGGCGAC
OptTy1-VHL Amino acid sequence (SEQ ID NO: 8)
QVQLVETGGGLVQPGGSLRLSCAASGFTFSSVYMNWVRQAPGKGPEWVSRISP
NSGNIGYTDSVKGRFTISRDNAKNTLYLQMNNLKPEDTALYYCAIGLNLSSSSVR
GQGTQVTVSSGSTLPVYTLKERCLQVVRSLVKPENYRRLDIVRSLYEDLEDHPNVQKDLE
RLTQERIAHQRMGD*
*indicates portion at end of sequence may optionally further include the following sequence
of digestion and protein tag for protein detection (as shown in SEQ ID NO: 9):
GTKLGPEQKLISEEDLNSAVDHHHHHH (SEQ ID NO: 10)
SEQ ID NO: 9:
QVQLVETGGGLVQPGGSLRLSCAASGFTFSSVYMNWVRQAPGKGPEWVSRISP
NSGNIGYTDSVKGRFTISRDNAKNTLYLQMNNLKPEDTALYYCAIGLNLSSSSVR
GQGTQVTVSSGSTLPVYTLKERCLQVVRSLVKPENYRRLDIVRSLYEDLEDHPNVQKDLE
RLTQERIAHQRMGDGTKLGPEQKLISEEDLNSAVDHHHHHH
The sequence bolded above in SEQ ID NO: 8 is SEQ ID NO: 11, and the sequence in
italics above in SEQ ID NO: 8 is SEQ ID NO: 12, as shown below.
SEQ ID NO: 11
**QVQLVETGGGLVQPGGSLRLSCAASGFTFSSVYMNWVRQAPGKGPEWVSRIS
PNSGNIGYTDSVKGRFTISRDNAKNTLYLQMNNLKPEDTALYYCAIGLNLSSSSV
RGQGTQVTVSS
**indicates one or more amino acid(s) may be optionally included. The amino acids include,
but are not limited to, natural amino acids such as M (as in SEQ ID NO: 64) or GS. Such an
embodiment includes, for example, SEQ ID NO: 13
MQVQLVETGGGLVQPGGSLRLSCAASGFTFSSVYMNWVRQAPGKGPEWVSRIS
PNSGNIGYTDSVKGRFTISRDNAKNTLYLQMNNLKPEDTALYYCAIGLNLSSSSV
RGQGTQVTVSSGSTLPVYTLKERCLQVVRSLVKPENYRRLDIVRSLYEDLEDHPNVQKDL
ERLTQERIAHQRMGD*
*indicates portion at end of sequence may optionally further include the sequence of SEQ ID
NO: 10, as shown in SEQ ID NO: 14 below:
MQVQLVETGGGLVQPGGSLRLSCAASGFTFSSVYMNWVRQAPGKGPEWVSRIS
PNSGNIGYTDSVKGRFTISRDNAKNTLYLQMNNLKPEDTALYYCAIGLNLSSSSV
RGQGTQVTVSSGSTLPVYTLKERCLQVVRSLVKPENYRRLDIVRSLYEDLEDHPNVQKDL
ERLTQERIAHQRMGDGTKLGPEQKLISEEDLNSAVDHHHHHH
SEQ ID NO: 12
GSTLPVYTLKERCLQVVRSLVKPENYRRLDIVRSLYEDLEDHPNVQKDLERLTQERIAHQR
MGD
2. Chimera 2 (Lane 3)
OptH11-H4-VHL DNA sequence (SEQ ID NO: 15)
CAAGTGCAGCTGGTGGAAAGCGGCGGAGGCCTGATGCAGGCTGGCGGCAG
CCTCAGACTGAGCTGCGCCGTCAGCGGCCGGACCTTCTCTACAGCCGCCAT
GGGCTGGTTTAGACAGGCCCCTGGCAAGGAACGCGAGTTCGTGGCCGCTAT
CCGGTGGTCCGGCGGATCTGCCTACTACGCTGATAGCGTGAAGGGCAGATT
CACCATCAGCAGAGATAAGGCCAAGAACACCGTGTACCTGCAGATGAACAG
CCTGAAATACGAGGACACCGCCGTGTACTATTGTGCCCAGACCCACTACGT
GTCCTACCTGCTGAGCGACTACGCCACCTGGCCCTACGACTACTGGGGACA
GGGCACACAGGTTACAGTGTCTAGCAAGGGATCCACACTGCCTGTGTACACCCT
GAAAGAGCGGTGCCTGCAAGTGGTCAGATCTCTCGTGAAGCCTGAGAACTACAGAAGA
CTGGACATCGTGCGGAGCCTGTACGAGGATCTGGAAGATCACCCCAACGTGCAGAAG
GACCTGGAACGGCTGACCCAGGAGAGAATCGCCCACCAGAGAATGGGCGAC*
*indicates portion at end of sequence may optionally further include SEQ ID NO: 3 (as
shown in SEQ ID NO: 16).
SEQ ID NO: 16
CAAGTGCAGCTGGTGGAAAGCGGCGGAGGCCTGATGCAGGCTGGCGGCAG
CCTCAGACTGAGCTGCGCCGTCAGCGGCCGGACCTTCTCTACAGCCGCCAT
GGGCTGGTTTAGACAGGCCCCTGGCAAGGAACGCGAGTTCGTGGCCGCTAT
CCGGTGGTCCGGCGGATCTGCCTACTACGCTGATAGCGTGAAGGGCAGATT
CACCATCAGCAGAGATAAGGCCAAGAACACCGTGTACCTGCAGATGAACAG
CCTGAAATACGAGGACACCGCCGTGTACTATTGTGCCCAGACCCACTACGT
GTCCTACCTGCTGAGCGACTACGCCACCTGGCCCTACGACTACTGGGGACA
GGGCACACAGGTTACAGTGTCTAGCAAGGGATCCACACTGCCTGTGTACACCCT
GAAAGAGCGGTGCCTGCAAGTGGTCAGATCTCTCGTGAAGCCTGAGAACTACAGAAGA
CTGGACATCGTGCGGAGCCTGTACGAGGATCTGGAAGATCACCCCAACGTGCAGAAG
GACCTGGAACGGCTGACCCAGGAGAGAATCGCCCACCAGAGAATGGGCGACGGTAC
CAAGCTTGGGCCCGAACAAAAACTCATCTCAGAAGAGGATCTGAATAGCGCCGT
CGACCATCATCATCATCATCATTGA
The sequence bolded above in SEQ ID NO: 15 is SEQ ID NO: 17 as shown below,
and the sequence in italics above in SEQ ID NO: 15 is SEQ ID NO: 5.
SEQ ID NO: 17
**CAAGTGCAGCTGGTGGAAAGCGGCGGAGGCCTGATGCAGGCTGGCGGCA
GCCTCAGACTGAGCTGCGCCGTCAGCGGCCGGACCTTCTCTACAGCCGCCA
TGGGCTGGTTTAGACAGGCCCCTGGCAAGGAACGCGAGTTCGTGGCCGCTA
TCCGGTGGTCCGGCGGATCTGCCTACTACGCTGATAGCGTGAAGGGCAGAT
TCACCATCAGCAGAGATAAGGCCAAGAACACCGTGTACCTGCAGATGAACA
GCCTGAAATACGAGGACACCGCCGTGTACTATTGTGCCCAGACCCACTACG
TGTCCTACCTGCTGAGCGACTACGCCACCTGGCCCTACGACTACTGGGGAC
AGGGCACACAGGTTACAGTGTCTAGCAAG
**indicates a nucleotide sequence encoding one or more amino acids may be optionally
included. For example, the nucleotide sequence may be ATG (as in SEQ ID NO: 65) or
GGATCC. Such an embodiment includes, for example, SEQ ID NO: 18
ATGCAAGTGCAGCTGGTGGAAAGCGGCGGAGGCCTGATGCAGGCTGGCGG
CAGCCTCAGACTGAGCTGCGCCGTCAGCGGCCGGACCTTCTCTACAGCCGC
CATGGGCTGGTTTAGACAGGCCCCTGGCAAGGAACGCGAGTTCGTGGCCGC
TATCCGGTGGTCCGGCGGATCTGCCTACTACGCTGATAGCGTGAAGGGCAG
ATTCACCATCAGCAGAGATAAGGCCAAGAACACCGTGTACCTGCAGATGAA
CAGCCTGAAATACGAGGACACCGCCGTGTACTATTGTGCCCAGACCCACTA
CGTGTCCTACCTGCTGAGCGACTACGCCACCTGGCCCTACGACTACTGGGG
ACAGGGCACACAGGTTACAGTGTCTAGCAAGGGATCCACACTGCCTGTGTACAC
CCTGAAAGAGCGGTGCCTGCAAGTGGTCAGATCTCTCGTGAAGCCTGAGAACTACAGA
AGACTGGACATCGTGCGGAGCCTGTACGAGGATCTGGAAGATCACCCCAACGTGCAG
AAGGACCTGGAACGGCTGACCCAGGAGAGAATCGCCCACCAGAGAATGGGCGAC*
*indicates portion at end of sequence may optionally further include SEQ ID NO: 3 as
shown in SEQ ID NO: 19
ATGCAAGTGCAGCTGGTGGAAAGCGGCGGAGGCCTGATGCAGGCTGGCGG
CAGCCTCAGACTGAGCTGCGCCGTCAGCGGCCGGACCTTCTCTACAGCCGC
CATGGGCTGGTTTAGACAGGCCCCTGGCAAGGAACGCGAGTTCGTGGCCGC
TATCCGGTGGTCCGGCGGATCTGCCTACTACGCTGATAGCGTGAAGGGCAG
ATTCACCATCAGCAGAGATAAGGCCAAGAACACCGTGTACCTGCAGATGAA
CAGCCTGAAATACGAGGACACCGCCGTGTACTATTGTGCCCAGACCCACTA
CGTGTCCTACCTGCTGAGCGACTACGCCACCTGGCCCTACGACTACTGGGG
ACAGGGCACACAGGTTACAGTGTCTAGCAAGGGATCCACACTGCCTGTGTACAC
CCTGAAAGAGCGGTGCCTGCAAGTGGTCAGATCTCTCGTGAAGCCTGAGAACTACAGA
AGACTGGACATCGTGCGGAGCCTGTACGAGGATCTGGAAGATCACCCCAACGTGCAG
AAGGACCTGGAACGGCTGACCCAGGAGAGAATCGCCCACCAGAGAATGGGCGACGG
TACCAAGCTTGGGCCCGAACAAAAACTCATCTCAGAAGAGGATCTGAATAGCGC
CGTCGACCATCATCATCATCATCATTGA
OptH11-H4-VHL Amino acid sequence (SEQ ID NO: 20)
QVQLVESGGGLMQAGGSLRLSCAVSGRTFSTAAMGWFRQAPGKEREFVAAIR
WSGGSAYYADSVKGRFTISRDKAKNTVYLQMNSLKYEDTAVYYCAQTHYVSYL
LSDYATWPYDYWGQGTQVTVSSKGSTLPVYTLKERCLQVVRSLVKPENYRRLDIVRSL
YEDLEDHPNVQKDLERLTQERIAHQRMGD*
*indicates portion at end of sequence may optionally further include SEQ ID NO: 10 (as
shown in SEQ ID NO: 21):
QVQLVESGGGLMQAGGSLRLSCAVSGRTFSTAAMGWFRQAPGKEREFVAAIR
WSGGSAYYADSVKGRFTISRDKAKNTVYLQMNSLKYEDTAVYYCAQTHYVSYL
LSDYATWPYDYWGQGTQVTVSSKGSTLPVYTLKERCLQVVRSLVKPENYRRLDIVRSL
YEDLEDHPNVQKDLERLTQERIAHQRMGDGTKLGPEQKLISEEDLNSAVDHHHHHH
The sequence bolded above in SEQ ID NO: 20 is SEQ ID NO: 22 as shown below,
and the sequence in italics above in SEQ ID NO: 20 is SEQ ID NO: 12.
SEQ ID NO: 22
**QVQLVESGGGLMQAGGSLRLSCAVSGRTFSTAAMGWFRQAPGKEREFVAAI
RWSGGSAYYADSVKGRFTISRDKAKNTVYLQMNSLKYEDTAVYYCAQTHYVSY
LLSDYATWPYDYWGQGTQVTVSSK
**indicates one or more amino acid(s) may be optionally included. The amino acids include,
but are not limited to, natural amino acids such as M (as in SEQ ID NO: 66) or GS. Such an
embodiment includes, for example, SEQ ID NO: 23
MQVQLVESGGGLMQAGGSLRLSCAVSGRTFSTAAMGWFRQAPGKEREFVAAI
RWSGGSAYYADSVKGRFTISRDKAKNTVYLQMNSLKYEDTAVYYCAQTHYVSY
LLSDYATWPYDYWGQGTQVTVSSKGSTLPVYTLKERCLQVVRSLVKPENYRRLDIVRS
LYEDLEDHPNVQKDLERLTQERIAHQRMGD*
*indicates portion at end of sequence may optionally further include SEQ ID NO: 10 (as
shown in SEQ ID NO: 24
MQVQLVESGGGLMQAGGSLRLSCAVSGRTFSTAAMGWFRQAPGKEREFVAAI
RWSGGSAYYADSVKGRFTISRDKAKNTVYLQMNSLKYEDTAVYYCAQTHYVSY
LLSDYATWPYDYWGQGTQVTVSSKGSTLPVYTLKERCLQVVRSLVKPENYRRLDIVRS
LYEDLEDHPNVQKDLERLTQERIAHQRMGDGTKLGPEQKLISEEDLNSAVDHHHHHH
3. Chimera 3 (Lane 4)
OptTy1-CHIP DNA sequence (SEQ ID NO: 25)
CAAGTGCAGCTGGTGGAAACCGGCGGCGGACTCGTGCAGCCTGGCGGATCT
CTGCGGCTGAGCTGTGCCGCTTCTGGCTTTACATTCAGCAGCGTGTACATGA
ATTGGGTCAGACAGGCCCCTGGAAAAGGCCCTGAGTGGGTGTCTAGAATCT
CCCCAAACAGCGGCAACATCGGCTACACCGACAGCGTGAAGGGCAGATTCA
CCATCAGCAGAGATAATGCCAAGAACACCCTGTATCTGCAGATGAACAACCT
GAAGCCCGAGGACACCGCCCTGTACTACTGCGCCATCGGCCTGAACCTGAG
CTCCAGCAGCGTGCGGGGCCAGGGCACACAGGTTACAGTGTCCAGCGAATTC
CGCCTCAACTTCGGCGATGATATTCCATCTGCCCTGAGAATCGCTAAGAAGAAAAGAT
GGAACAGCATCGAGGAAAGGCGGATCCACCAGGAGAGCGAGCTGCACAGCTACCTGA
GCAGACTGATCGCCGCTGAACGGGAAAGAGAACTGGAAGAGTGCCAGAGAAACCACG
AGGGCGACGAGGACGACAGCCACGTGCGGGCCCAGCAGGCCTGCATCGAGGCCAAG
CACGATAAGTACATGGCCGACATGGACGAACTGTTCAGCCAGGTCGACGAGAAGCGG
AAGAAGCGGGACATCCCTGATTATCTGTGCGGCAAGATCTCTTTTGAGCTGATGAGAG
AGCCTTGTATCACCCCTTCCGGCATCACCTACGACAGAAAGGACATCGAGGAACACCT
GCAAAGAGTGGGACATTTCGACCCCGTGACAAGAAGCCCTCTGACCCAGGAGCAGCT
GATCCCCAATCTGGCCATGAAAGAGGTGATCGACGCCTTCATCTCCGAGAACGGCTGG
GTGGAAGATTAC*
*indicates portion at end of sequence may optionally further include SEQ ID NO: 3 (as
shown in SEQ ID NO: 26):
CAAGTGCAGCTGGTGGAAACCGGCGGCGGACTCGTGCAGCCTGGCGGATCT
CTGCGGCTGAGCTGTGCCGCTTCTGGCTTTACATTCAGCAGCGTGTACATGA
ATTGGGTCAGACAGGCCCCTGGAAAAGGCCCTGAGTGGGTGTCTAGAATCT
CCCCAAACAGCGGCAACATCGGCTACACCGACAGCGTGAAGGGCAGATTCA
CCATCAGCAGAGATAATGCCAAGAACACCCTGTATCTGCAGATGAACAACCT
GAAGCCCGAGGACACCGCCCTGTACTACTGCGCCATCGGCCTGAACCTGAG
CTCCAGCAGCGTGCGGGGCCAGGGCACACAGGTTACAGTGTCCAGCGAATTC
CGCCTCAACTTCGGCGATGATATTCCATCTGCCCTGAGAATCGCTAAGAAGAAAAGAT
GGAACAGCATCGAGGAAAGGCGGATCCACCAGGAGAGCGAGCTGCACAGCTACCTGA
GCAGACTGATCGCCGCTGAACGGGAAAGAGAACTGGAAGAGTGCCAGAGAAACCACG
AGGGCGACGAGGACGACAGCCACGTGCGGGCCCAGCAGGCCTGCATCGAGGCCAAG
CACGATAAGTACATGGCCGACATGGACGAACTGTTCAGCCAGGTCGACGAGAAGCGG
AAGAAGCGGGACATCCCTGATTATCTGTGCGGCAAGATCTCTTTTGAGCTGATGAGAG
AGCCTTGTATCACCCCTTCCGGCATCACCTACGACAGAAAGGACATCGAGGAACACCT
GCAAAGAGTGGGACATTTCGACCCCGTGACAAGAAGCCCTCTGACCCAGGAGCAGCT
GATCCCCAATCTGGCCATGAAAGAGGTGATCGACGCCTTCATCTCCGAGAACGGCTGG
GTGGAAGATTACGGTACCAAGCTTGGGCCCGAACAAAAACTCATCTCAGAAGAGG
ATCTGAATAGCGCCGTCGACCATCATCATCATCATCATTGA
The sequence bolded above in SEQ ID NO: 25 is SEQ ID NO: 4, and the sequence in
italics above in SEQ ID NO: 25 is SEQ ID NO: 27.
SEQ ID NO: 4
**CAAGTGCAGCTGGTGGAAACCGGCGGCGGACTCGTGCAGCCTGGCGGAT
CTCTGCGGCTGAGCTGTGCCGCTTCTGGCTTTACATTCAGCAGCGTGTACAT
GAATTGGGTCAGACAGGCCCCTGGAAAAGGCCCTGAGTGGGTGTCTAGAAT
CTCCCCAAACAGCGGCAACATCGGCTACACCGACAGCGTGAAGGGCAGATT
CACCATCAGCAGAGATAATGCCAAGAACACCCTGTATCTGCAGATGAACAAC
CTGAAGCCCGAGGACACCGCCCTGTACTACTGCGCCATCGGCCTGAACCTG
AGCTCCAGCAGCGTGCGGGGCCAGGGCACACAGGTTACAGTGTCCAGC
*indicates a nucleotide sequence encoding one or more amino acids may be optionally
included. For example, the nucleotide sequence may be ATG (as in SEQ ID NO: 63) or
GGATCC. Such an embodiment includes, for example, SEQ ID NO: 28
ATGCAAGTGCAGCTGGTGGAAACCGGCGGCGGACTCGTGCAGCCTGGCGG
ATCTCTGCGGCTGAGCTGTGCCGCTTCTGGCTTTACATTCAGCAGCGTGTAC
ATGAATTGGGTCAGACAGGCCCCTGGAAAAGGCCCTGAGTGGGTGTCTAGA
ATCTCCCCAAACAGCGGCAACATCGGCTACACCGACAGCGTGAAGGGCAGA
TTCACCATCAGCAGAGATAATGCCAAGAACACCCTGTATCTGCAGATGAACA
ACCTGAAGCCCGAGGACACCGCCCTGTACTACTGCGCCATCGGCCTGAACC
TGAGCTCCAGCAGCGTGCGGGGCCAGGGCACACAGGTTACAGTGTCCAGCG
AATTCCGCCTCAACTTCGGCGATGATATTCCATCTGCCCTGAGAATCGCTAAGAAGAAA
AGATGGAACAGCATCGAGGAAAGGCGGATCCACCAGGAGAGCGAGCTGCACAGCTAC
CTGAGCAGACTGATCGCCGCTGAACGGGAAAGAGAACTGGAAGAGTGCCAGAGAAAC
CACGAGGGCGACGAGGACGACAGCCACGTGCGGGCCCAGCAGGCCTGCATCGAGGC
CAAGCACGATAAGTACATGGCCGACATGGACGAACTGTTCAGCCAGGTCGACGAGAA
GCGGAAGAAGCGGGACATCCCTGATTATCTGTGCGGCAAGATCTCTTTTGAGCTGATG
AGAGAGCCTTGTATCACCCCTTCCGGCATCACCTACGACAGAAAGGACATCGAGGAAC
ACCTGCAAAGAGTGGGACATTTCGACCCCGTGACAAGAAGCCCTCTGACCCAGGAGC
AGCTGATCCCCAATCTGGCCATGAAAGAGGTGATCGACGCCTTCATCTCCGAGAACGG
CTGGGTGGAAGATTAC*
*indicates portion at end of sequence may optionally further include SEQ ID NO: 3 (as
shown in SEQ ID NO: 29
ATGCAAGTGCAGCTGGTGGAAACCGGCGGCGGACTCGTGCAGCCTGGCGG
ATCTCTGCGGCTGAGCTGTGCCGCTTCTGGCTTTACATTCAGCAGCGTGTAC
ATGAATTGGGTCAGACAGGCCCCTGGAAAAGGCCCTGAGTGGGTGTCTAGA
ATCTCCCCAAACAGCGGCAACATCGGCTACACCGACAGCGTGAAGGGCAGA
TTCACCATCAGCAGAGATAATGCCAAGAACACCCTGTATCTGCAGATGAACA
ACCTGAAGCCCGAGGACACCGCCCTGTACTACTGCGCCATCGGCCTGAACC
TGAGCTCCAGCAGCGTGCGGGGCCAGGGCACACAGGTTACAGTGTCCAGCG
AATTCCGCCTCAACTTCGGCGATGATATTCCATCTGCCCTGAGAATCGCTAAGAAGAAA
AGATGGAACAGCATCGAGGAAAGGCGGATCCACCAGGAGAGCGAGCTGCACAGCTAC
CTGAGCAGACTGATCGCCGCTGAACGGGAAAGAGAACTGGAAGAGTGCCAGAGAAAC
CACGAGGGCGACGAGGACGACAGCCACGTGCGGGCCCAGCAGGCCTGCATCGAGGC
CAAGCACGATAAGTACATGGCCGACATGGACGAACTGTTCAGCCAGGTCGACGAGAA
GCGGAAGAAGCGGGACATCCCTGATTATCTGTGCGGCAAGATCTCTTTTGAGCTGATG
AGAGAGCCTTGTATCACCCCTTCCGGCATCACCTACGACAGAAAGGACATCGAGGAAC
ACCTGCAAAGAGTGGGACATTTCGACCCCGTGACAAGAAGCCCTCTGACCCAGGAGC
AGCTGATCCCCAATCTGGCCATGAAAGAGGTGATCGACGCCTTCATCTCCGAGAACGG
CTGGGTGGAAGATTACGGTACCAAGCTTGGGCCCGAACAAAAACTCATCTCAGAA
GAGGATCTGAATAGCGCCGTCGACCATCATCATCATCATCATTGA
SEQ ID NO: 27
GAATTCCGCCTCAACTTCGGCGATGATATTCCATCTGCCCTGAGAATCGCTAAGAAGAA
AAGATGGAACAGCATCGAGGAAAGGCGGATCCACCAGGAGAGCGAGCTGCACAGCTA
CCTGAGCAGACTGATCGCCGCTGAACGGGAAAGAGAACTGGAAGAGTGCCAGAGAAA
CCACGAGGGCGACGAGGACGACAGCCACGTGCGGGCCCAGCAGGCCTGCATCGAGG
CCAAGCACGATAAGTACATGGCCGACATGGACGAACTGTTCAGCCAGGTCGACGAGA
AGCGGAAGAAGCGGGACATCCCTGATTATCTGTGCGGCAAGATCTCTTTTGAGCTGAT
GAGAGAGCCTTGTATCACCCCTTCCGGCATCACCTACGACAGAAAGGACATCGAGGAA
CACCTGCAAAGAGTGGGACATTTCGACCCCGTGACAAGAAGCCCTCTGACCCAGGAG
CAGCTGATCCCCAATCTGGCCATGAAAGAGGTGATCGACGCCTTCATCTCCGAGAACG
GCTGGGTGGAAGATTAC
OptTy1-CHIP Amino acid sequence (SEQ ID NO: 30)
QVQLVETGGGLVQPGGSLRLSCAASGFTFSSVYMNWVRQAPGKGPEWVSRISP
NSGNIGYTDSVKGRFTISRDNAKNTLYLQMNNLKPEDTALYYCAIGLNLSSSSVR
GQGTQVTVSSEFRLNFGDDIPSALRIAKKKRWNSIEERRIHQESELHSYLSRLIAAEREREL
EECQRNHEGDEDDSHVRAQQACIEAKHDKYMADMDELFSQVDEKRKKRDIPDYLCGKI
SFELMREPCITPSGITYDRKDIEEHLQRVGHFDPVTRSPLTQEQLIPNLAMKEVIDAFISEN
GWVEDY*
*indicates portion at end of sequence may optionally further include SEQ ID NO: 10 (as
shown in SEQ ID NO: 31):
QVQLVETGGGLVQPGGSLRLSCAASGFTFSSVYMNWVRQAPGKGPEWVSRISP
NSGNIGYTDSVKGRFTISRDNAKNTLYLQMNNLKPEDTALYYCAIGLNLSSSSVR
GQGTQVTVSSEFRLNFGDDIPSALRIAKKKRWNSIEERRIHQESELHSYLSRLIAAEREREL
EECQRNHEGDEDDSHVRAQQACIEAKHDKYMADMDELFSQVDEKRKKRDIPDYLCGKI
SFELMREPCITPSGITYDRKDIEEHLQRVGHFDPVTRSPLTQEQLIPNLAMKEVIDAFISEN
GWVEDYGTKLGPEQKLISEEDLNSAVDHHHHHH
The sequence bolded above in SEQ ID NO: 30 is SEQ ID NO: 11, and the sequence
in italics above in SEQ ID NO: 30 is SEQ ID NO: 32, as shown below.
SEQ ID NO: 11
**QVQLVETGGGLVQPGGSLRLSCAASGFTFSSVYMNWVRQAPGKGPEWVSRIS
PNSGNIGYTDSVKGRFTISRDNAKNTLYLQMNNLKPEDTALYYCAIGLNLSSSSV
RGQGTQVTVSS
**indicates one or more amino acid(s) may be optionally included. The amino acids include,
but are not limited to, natural amino acids such as M (as in SEQ ID NO: 64) or GS. Such an
embodiment includes, for example, SEQ ID NO: 33
MQVQLVETGGGLVQPGGSLRLSCAASGFTFSSVYMNWVRQAPGKGPEWVSRIS
PNSGNIGYTDSVKGRFTISRDNAKNTLYLQMNNLKPEDTALYYCAIGLNLSSSSV
RGQGTQVTVSSEFRLNFGDDIPSALRIAKKKRWNSIEERRIHQESELHSYLSRLIAAERER
ELEECQRNHEGDEDDSHVRAQQACIEAKHDKYMADMDELFSQVDEKRKKRDIPDYLCG
KISFELMREPCITPSGITYDRKDIEEHLQRVGHFDPVTRSPLTQEQLIPNLAMKEVIDAFISE
NGWVEDY*
*indicates portion at end of sequence may optionally further include SEQ ID NO: 10 (as
shown in SEQ ID NO: 34
MQVQLVETGGGLVQPGGSLRLSCAASGFTFSSVYMNWVRQAPGKGPEWVSRIS
PNSGNIGYTDSVKGRFTISRDNAKNTLYLQMNNLKPEDTALYYCAIGLNLSSSSV
RGQGTQVTVSSEFRLNFGDDIPSALRIAKKKRWNSIEERRIHQESELHSYLSRLIAAERER
ELEECQRNHEGDEDDSHVRAQQACIEAKHDKYMADMDELFSQVDEKRKKRDIPDYLCG
KISFELMREPCITPSGITYDRKDIEEHLQRVGHFDPVTRSPLTQEQLIPNLAMKEVIDAFISE
NGWVEDYGTKLGPEQKLISEEDLNSAVDHHHHHH
SEQ ID NO: 32
EFRLNFGDDIPSALRIAKKKRWNSIEERRIHQESELHSYLSRLIAAERERELEECQRNHEGD
EDDSHVRAQQACIEAKHDKYMADMDELFSQVDEKRKKRDIPDYLCGKISFELMREPCITP
SGITYDRKDIEEHLQRVGHFDPVTRSPLTQEQLIPNLAMKEVIDAFISENGWVEDY
4. Chimera 4 (Lane 5)
OptH11-H4-CHIP DNA sequence (SEQ ID NO: 35)
CAAGTGCAGCTGGTGGAAAGCGGCGGAGGCCTGATGCAGGCTGGCGGCAG
CCTCAGACTGAGCTGCGCCGTCAGCGGCCGGACCTTCTCTACAGCCGCCAT
GGGCTGGTTTAGACAGGCCCCTGGCAAGGAACGCGAGTTCGTGGCCGCTAT
CCGGTGGTCCGGCGGATCTGCCTACTACGCTGATAGCGTGAAGGGCAGATT
CACCATCAGCAGAGATAAGGCCAAGAACACCGTGTACCTGCAGATGAACAG
CCTGAAATACGAGGACACCGCCGTGTACTATTGTGCCCAGACCCACTACGT
GTCCTACCTGCTGAGCGACTACGCCACCTGGCCCTACGACTACTGGGGACA
GGGCACACAGGTTACAGTGTCTAGCAAGGAATTCCGCCTCAACTTCGGCGATGA
TATTCCATCTGCCCTGAGAATCGCTAAGAAGAAAAGATGGAACAGCATCGAGGAAAGG
CGGATCCACCAGGAGAGCGAGCTGCACAGCTACCTGAGCAGACTGATCGCCGCTGAA
CGGGAAAGAGAACTGGAAGAGTGCCAGAGAAACCACGAGGGCGACGAGGACGACAG
CCACGTGCGGGCCCAGCAGGCCTGCATCGAGGCCAAGCACGATAAGTACATGGCCGA
CATGGACGAACTGTTCAGCCAGGTCGACGAGAAGCGGAAGAAGCGGGACATCCCTGA
TTATCTGTGCGGCAAGATCTCTTTTGAGCTGATGAGAGAGCCTTGTATCACCCCTTCCG
GCATCACCTACGACAGAAAGGACATCGAGGAACACCTGCAAAGAGTGGGACATTTCGA
CCCCGTGACAAGAAGCCCTCTGACCCAGGAGCAGCTGATCCCCAATCTGGCCATGAA
AGAGGTGATCGACGCCTTCATCTCCGAGAACGGCTGGGTGGAAGATTAC*
*indicates portion at end of sequence may optionally further include SEQ ID NO: 3 as
shown in SEQ ID NO 36:
CAAGTGCAGCTGGTGGAAAGCGGCGGAGGCCTGATGCAGGCTGGCGGCAG
CCTCAGACTGAGCTGCGCCGTCAGCGGCCGGACCTTCTCTACAGCCGCCAT
GGGCTGGTTTAGACAGGCCCCTGGCAAGGAACGCGAGTTCGTGGCCGCTAT
CCGGTGGTCCGGCGGATCTGCCTACTACGCTGATAGCGTGAAGGGCAGATT
CACCATCAGCAGAGATAAGGCCAAGAACACCGTGTACCTGCAGATGAACAG
CCTGAAATACGAGGACACCGCCGTGTACTATTGTGCCCAGACCCACTACGT
GTCCTACCTGCTGAGCGACTACGCCACCTGGCCCTACGACTACTGGGGACA
GGGCACACAGGTTACAGTGTCTAGCAAGGAATTCCGCCTCAACTTCGGCGATGA
TATTCCATCTGCCCTGAGAATCGCTAAGAAGAAAAGATGGAACAGCATCGAGGAAAGG
CGGATCCACCAGGAGAGCGAGCTGCACAGCTACCTGAGCAGACTGATCGCCGCTGAA
CGGGAAAGAGAACTGGAAGAGTGCCAGAGAAACCACGAGGGCGACGAGGACGACAG
CCACGTGCGGGCCCAGCAGGCCTGCATCGAGGCCAAGCACGATAAGTACATGGCCGA
CATGGACGAACTGTTCAGCCAGGTCGACGAGAAGCGGAAGAAGCGGGACATCCCTGA
TTATCTGTGCGGCAAGATCTCTTTTGAGCTGATGAGAGAGCCTTGTATCACCCCTTCCG
GCATCACCTACGACAGAAAGGACATCGAGGAACACCTGCAAAGAGTGGGACATTTCGA
CCCCGTGACAAGAAGCCCTCTGACCCAGGAGCAGCTGATCCCCAATCTGGCCATGAA
AGAGGTGATCGACGCCTTCATCTCCGAGAACGGCTGGGTGGAAGATTACGGTACCAA
GCTTGGGCCCGAACAAAAACTCATCTCAGAAGAGGATCTGAATAGCGCCGTCGA
CCATCATCATCATCATCATTGA
The sequence bolded above in SEQ ID NO: 35 is SEQ ID NO: 17 as shown below,
and the sequence in italics above in SEQ ID NO: 35 is SEQ ID NO: 27.
SEQ ID NO: 17
**CAAGTGCAGCTGGTGGAAAGCGGCGGAGGCCTGATGCAGGCTGGCGGCA
GCCTCAGACTGAGCTGCGCCGTCAGCGGCCGGACCTTCTCTACAGCCGCCA
TGGGCTGGTTTAGACAGGCCCCTGGCAAGGAACGCGAGTTCGTGGCCGCTA
TCCGGTGGTCCGGCGGATCTGCCTACTACGCTGATAGCGTGAAGGGCAGAT
TCACCATCAGCAGAGATAAGGCCAAGAACACCGTGTACCTGCAGATGAACA
GCCTGAAATACGAGGACACCGCCGTGTACTATTGTGCCCAGACCCACTACG
TGTCCTACCTGCTGAGCGACTACGCCACCTGGCCCTACGACTACTGGGGAC
AGGGCACACAGGTTACAGTGTCTAGCAAG
**indicates a nucleotide sequence encoding one or more amino acids may be optionally
included. For example, the nucleotide sequence may be ATG (as in SEQ ID NO: 65) or
GGATCC. Such an embodiment includes, for example, SEQ ID NO: 37:
ATGCAAGTGCAGCTGGTGGAAAGCGGCGGAGGCCTGATGCAGGCTGGCGG
CAGCCTCAGACTGAGCTGCGCCGTCAGCGGCCGGACCTTCTCTACAGCCGC
CATGGGCTGGTTTAGACAGGCCCCTGGCAAGGAACGCGAGTTCGTGGCCGC
TATCCGGTGGTCCGGCGGATCTGCCTACTACGCTGATAGCGTGAAGGGCAG
ATTCACCATCAGCAGAGATAAGGCCAAGAACACCGTGTACCTGCAGATGAA
CAGCCTGAAATACGAGGACACCGCCGTGTACTATTGTGCCCAGACCCACTA
CGTGTCCTACCTGCTGAGCGACTACGCCACCTGGCCCTACGACTACTGGGG
ACAGGGCACACAGGTTACAGTGTCTAGCAAGGAATTCCGCCTCAACTTCGGCGA
TGATATTCCATCTGCCCTGAGAATCGCTAAGAAGAAAAGATGGAACAGCATCGAGGAA
AGGCGGATCCACCAGGAGAGCGAGCTGCACAGCTACCTGAGCAGACTGATCGCCGCT
GAACGGGAAAGAGAACTGGAAGAGTGCCAGAGAAACCACGAGGGCGACGAGGACGA
CAGCCACGTGCGGGCCCAGCAGGCCTGCATCGAGGCCAAGCACGATAAGTACATGGC
CGACATGGACGAACTGTTCAGCCAGGTCGACGAGAAGCGGAAGAAGCGGGACATCCC
TGATTATCTGTGCGGCAAGATCTCTTTTGAGCTGATGAGAGAGCCTTGTATCACCCCTT
CCGGCATCACCTACGACAGAAAGGACATCGAGGAACACCTGCAAAGAGTGGGACATTT
CGACCCCGTGACAAGAAGCCCTCTGACCCAGGAGCAGCTGATCCCCAATCTGGCCAT
GAAAGAGGTGATCGACGCCTTCATCTCCGAGAACGGCTGGGTGGAAGATTAC*
*indicates portion at end of sequence may optionally further include SEQ ID NO: 3 as
shown in SEQ ID NO: 38:
ATGCAAGTGCAGCTGGTGGAAAGCGGCGGAGGCCTGATGCAGGCTGGCGG
CAGCCTCAGACTGAGCTGCGCCGTCAGCGGCCGGACCTTCTCTACAGCCGC
CATGGGCTGGTTTAGACAGGCCCCTGGCAAGGAACGCGAGTTCGTGGCCGC
TATCCGGTGGTCCGGCGGATCTGCCTACTACGCTGATAGCGTGAAGGGCAG
ATTCACCATCAGCAGAGATAAGGCCAAGAACACCGTGTACCTGCAGATGAA
CAGCCTGAAATACGAGGACACCGCCGTGTACTATTGTGCCCAGACCCACTA
CGTGTCCTACCTGCTGAGCGACTACGCCACCTGGCCCTACGACTACTGGGG
ACAGGGCACACAGGTTACAGTGTCTAGCAAGGAATTCCGCCTCAACTTCGGCGA
TGATATTCCATCTGCCCTGAGAATCGCTAAGAAGAAAAGATGGAACAGCATCGAGGAA
AGGCGGATCCACCAGGAGAGCGAGCTGCACAGCTACCTGAGCAGACTGATCGCCGCT
GAACGGGAAAGAGAACTGGAAGAGTGCCAGAGAAACCACGAGGGCGACGAGGACGA
CAGCCACGTGCGGGCCCAGCAGGCCTGCATCGAGGCCAAGCACGATAAGTACATGGC
CGACATGGACGAACTGTTCAGCCAGGTCGACGAGAAGCGGAAGAAGCGGGACATCCC
TGATTATCTGTGCGGCAAGATCTCTTTTGAGCTGATGAGAGAGCCTTGTATCACCCCTT
CCGGCATCACCTACGACAGAAAGGACATCGAGGAACACCTGCAAAGAGTGGGACATTT
CGACCCCGTGACAAGAAGCCCTCTGACCCAGGAGCAGCTGATCCCCAATCTGGCCAT
GAAAGAGGTGATCGACGCCTTCATCTCCGAGAACGGCTGGGTGGAAGATTACGGTAC
CAAGCTTGGGCCCGAACAAAAACTCATCTCAGAAGAGGATCTGAATAGCGCCGT
CGACCATCATCATCATCATCATTGA
OptH11-H4-CHIP Amino acid sequence (SEQ ID NO: 39)
QVQLVESGGGLMQAGGSLRLSCAVSGRTFSTAAMGWFRQAPGKEREFVAAIR
WSGGSAYYADSVKGRFTISRDKAKNTVYLQMNSLKYEDTAVYYCAQTHYVSYL
LSDYATWPYDYWGQGTQVTVSSKEFRLNFGDDIPSALRIAKKKRWNSIEERRIHQESE
LHSYLSRLIAAERERELEECQRNHEGDEDDSHVRAQQACIEAKHDKYMADMDELFSQVD
EKRKKRDIPDYLCGKISFELMREPCITPSGITYDRKDIEEHLQRVGHFDPVTRSPLTQEQLI
PNLAMKEVIDAFISENGWVEDY*
*indicates portion at end of sequence may optionally further SEQ ID NO: 10 as shown in
SEQ ID NO 40:
QVQLVESGGGLMQAGGSLRLSCAVSGRTFSTAAMGWFRQAPGKEREFVAAIR
WSGGSAYYADSVKGRFTISRDKAKNTVYLQMNSLKYEDTAVYYCAQTHYVSYL
LSDYATWPYDYWGQGTQVTVSSKEFRLNFGDDIPSALRIAKKKRWNSIEERRIHQESE
LHSYLSRLIAAERERELEECQRNHEGDEDDSHVRAQQACIEAKHDKYMADMDELFSQVD
EKRKKRDIPDYLCGKISFELMREPCITPSGITYDRKDIEEHLQRVGHFDPVTRSPLTQEQLI
PNLAMKEVIDAFISENGWVEDYGTKLGPEQKLISEEDLNSAVDHHHHHH
The sequence bolded above in SEQ ID NO: 39 is SEQ ID NO: 22 as shown below,
and the sequence in italics above in SEQ ID NO: 39 is SEQ ID NO: 32.
SEQ ID NO: 22
**QVQLVESGGGLMQAGGSLRLSCAVSGRTFSTAAMGWFRQAPGKEREFVAAI
RWSGGSAYYADSVKGRFTISRDKAKNTVYLQMNSLKYEDTAVYYCAQTHYVSY
LLSDYATWPYDYWGQGTQVTVSSK
**indicates one or more amino acid(s) may be optionally included. The amino acids include,
but are not limited to, natural amino acids such as M (as in SEQ ID NO: 66) or GS. Such an
embodiment includes, for example, SEQ ID NO: 41:
MQVQLVESGGGLMQAGGSLRLSCAVSGRTFSTAAMGWFRQAPGKEREFVAAI
RWSGGSAYYADSVKGRFTISRDKAKNTVYLQMNSLKYEDTAVYYCAQTHYVSY
LLSDYATWPYDYWGQGTQVTVSSKEFRLNFGDDIPSALRIAKKKRWNSIEERRIHQES
ELHSYLSRLIAAERERELEECQRNHEGDEDDSHVRAQQACIEAKHDKYMADMDELFSQV
DEKRKKRDIPDYLCGKISFELMREPCITPSGITYDRKDIEEHLQRVGHFDPVTRSPLTQEQ
LIPNLAMKEVIDAFISENGWVEDY*
*indicates portion at end of sequence may optionally further SEQ ID NO: 10 as shown in
SEQ ID NO: 42
MQVQLVESGGGLMQAGGSLRLSCAVSGRTESTAAMGWFRQAPGKEREFVAAI
RWSGGSAYYADSVKGRFTISRDKAKNTVYLQMNSLKYEDTAVYYCAQTHYVSY
LLSDYATWPYDYWGQGTQVTVSSKEFRLNFGDDIPSALRIAKKKRWNSIEERRIHQES
ELHSYLSRLIAAERERELEECQRNHEGDEDDSHVRAQQACIEAKHDKYMADMDELFSQV
DEKRKKRDIPDYLCGKISFELMREPCITPSGITYDRKDIEEHLQRVGHFDPVTRSPLTQEQ
LIPNLAMKEVIDAFISENGWVEDY GTKLGPEQKLISEEDLNSAVDHHHHHH
5. Chimera 5 (Lane 6)
Beta-Trc-OptTy1 DNA sequence (SEQ ID NO: 43)
ATGGACCCTGCTGAGGCCGTGCTGCAAGAGAAGGCCCTGAAATTCATGAACAGCAGC
GAGCGGGAAGATTGCAACAACGGCGAACCTCCTAGAAAGATCATCCCCGAGAAGAATA
GCCTGAGACAGACCTACAACTCTTGCGCCCGCCTCTGTCTGAACCAGGAGACCGTGT
GCCTGGCTTCCACAGCTATGAAAACCGAGAATTGCGTGGCCAAGACCAAGCTTGCTAA
TGGCACATCTAGCATGATCGTGCCTAAGCAGAGAAAGCTGTCCGCCTCTTACGAGAAG
GAAAAAGAACTGTGCGTCAAGTACTTCGAGCAGTGGAGCGAGTCTGATCAGGTGGAAT
TCGTGGAACACCTGATCAGCCAGATGTGCCACTACCAGCACGGCCACATCAACAGCTA
CCTGAAGCCTATGCTGCAGAGAGACTTCATCACCGCCCTGCCTGCCAGAGGCCTGGA
CCATATCGCCGAGAACATCCTGAGCTACCTGGACGCCAAAAGCCTGTGTGCCGCCGA
GCTGGTGTGCAAGGAGTGGTATAGAGTGACAAGCGATGGAATGCTGTGGAAGAAGCT
GATTGAGAGAATGGTGCGGACCGACAGCCTGTGGCGGGGCCTGGCCGAACGGAGAG
GCTGGGGACAGTACCTGTTTAAGAACAAGCCCCCAGACGGCAACGCCCCTCCAAACA
GCTTCTACAGAGCCCTGTACCCCAAGATCATCCAGGACATCGAAACCATCGAGAGCAA
CTGGAGGTGTGGCCGGCACTCCCTGCAAAGAATCCACTGCAGACAAGTGCAGCTG
GTGGAAACCGGCGGCGGACTCGTGCAGCCTGGCGGATCTCTGCGGCTGAGC
TGTGCCGCTTCTGGCTTTACATTCAGCAGCGTGTACATGAATTGGGTCAGAC
AGGCCCCTGGAAAAGGCCCTGAGTGGGTGTCTAGAATCTCCCCAAACAGCG
GCAACATCGGCTACACCGACAGCGTGAAGGGCAGATTCACCATCAGCAGAG
ATAATGCCAAGAACACCCTGTATCTGCAGATGAACAACCTGAAGCCCGAGG
ACACCGCCCTGTACTACTGCGCCATCGGCCTGAACCTGAGCTCCAGCAGCG
TGCGGGGCCAGGGCACACAGGTTACAGTGTCCAGC* or
Beta-Trc-OptTy1 DNA sequence (SEQ ID NO: 44)
ATGGACCCTGCTGAGGCCGTGCTGCAAGAGAAGGCCCTGAAATTCATGAACAGCAGC
GAGCGGGAAGATTGCAACAACGGCGAACCTCCTAGAAAGATCATCCCCGAGAAGAATA
GCCTGAGACAGACCTACAACTCTTGCGCCCGCCTCTGTCTGAACCAGGAGACCGTGT
GCCTGGCTTCCACAGCTATGAAAACCGAGAATTGCGTGGCCAAGACCAAGCTTGCTAA
TGGCACATCTAGCATGATCGTGCCTAAGCAGAGAAAGCTGTCCGCCTCTTACGAGAAG
GAAAAAGAACTGTGCGTCAAGTACTTCGAGCAGTGGAGCGAGTCTGATCAGGTGGAAT
TCGTGGAACACCTGATCAGCCAGATGTGCCACTACCAGCACGGCCACATCAACAGCTA
CCTGAAGCCTATGCTGCAGAGAGACTTCATCACCGCCCTGCCTGCCAGAGGCCTGGA
CCATATCGCCGAGAACATCCTGAGCTACCTGGACGCCAAAAGCCTGTGTGCCGCCGA
GCTGGTGTGCAAGGAGTGGTATAGAGTGACAAGCGATGGAATGCTGTGGAAGAAGCT
GATTGAGAGAATGGTGCGGACCGACAGCCTGTGGCGGGGCCTGGCCGAACGGAGAG
GCTGGGGACAGTACCTGTTTAAGAACAAGCCCCCAGACGGCAACGCCCCTCCAAACA
GCTTCTACAGAGCCCTGTACCCCAAGATCATCCAGGACATCGAAACCATCGAGAGCAA
CTGGAGGTGTGGCCGGCACTCCCTGCAAAGAATCCACTGCAGAGGATCCCAAGTGC
AGCTGGTGGAAACCGGCGGCGGACTCGTGCAGCCTGGCGGATCTCTGCGGC
TGAGCTGTGCCGCTTCTGGCTTTACATTCAGCAGCGTGTACATGAATTGGGT
CAGACAGGCCCCTGGAAAAGGCCCTGAGTGGGTGTCTAGAATCTCCCCAAA
CAGCGGCAACATCGGCTACACCGACAGCGTGAAGGGCAGATTCACCATCAG
CAGAGATAATGCCAAGAACACCCTGTATCTGCAGATGAACAACCTGAAGCC
CGAGGACACCGCCCTGTACTACTGCGCCATCGGCCTGAACCTGAGCTCCAG
CAGCGTGCGGGGCCAGGGCACACAGGTTACAGTGTCCAGC*
*indicates portion at end of each of SEQ ID NO: 43 or SEQ ID NO: 44 may optionally
further include the in the sequence of SEQ ID NO: 3 as shown below in SEQ ID NOs: 45 and
46, respectively:
SEQ ID NO: 45
ATGGACCCTGCTGAGGCCGTGCTGCAAGAGAAGGCCCTGAAATTCATGAACAGCAGC
GAGCGGGAAGATTGCAACAACGGCGAACCTCCTAGAAAGATCATCCCCGAGAAGAATA
GCCTGAGACAGACCTACAACTCTTGCGCCCGCCTCTGTCTGAACCAGGAGACCGTGT
GCCTGGCTTCCACAGCTATGAAAACCGAGAATTGCGTGGCCAAGACCAAGCTTGCTAA
TGGCACATCTAGCATGATCGTGCCTAAGCAGAGAAAGCTGTCCGCCTCTTACGAGAAG
GAAAAAGAACTGTGCGTCAAGTACTTCGAGCAGTGGAGCGAGTCTGATCAGGTGGAAT
TCGTGGAACACCTGATCAGCCAGATGTGCCACTACCAGCACGGCCACATCAACAGCTA
CCTGAAGCCTATGCTGCAGAGAGACTTCATCACCGCCCTGCCTGCCAGAGGCCTGGA
CCATATCGCCGAGAACATCCTGAGCTACCTGGACGCCAAAAGCCTGTGTGCCGCCGA
GCTGGTGTGCAAGGAGTGGTATAGAGTGACAAGCGATGGAATGCTGTGGAAGAAGCT
GATTGAGAGAATGGTGCGGACCGACAGCCTGTGGCGGGGCCTGGCCGAACGGAGAG
GCTGGGGACAGTACCTGTTTAAGAACAAGCCCCCAGACGGCAACGCCCCTCCAAACA
GCTTCTACAGAGCCCTGTACCCCAAGATCATCCAGGACATCGAAACCATCGAGAGCAA
CTGGAGGTGTGGCCGGCACTCCCTGCAAAGAATCCACTGCAGACAAGTGCAGCTG
GTGGAAACCGGCGGCGGACTCGTGCAGCCTGGCGGATCTCTGCGGCTGAGC
TGTGCCGCTTCTGGCTTTACATTCAGCAGCGTGTACATGAATTGGGTCAGAC
AGGCCCCTGGAAAAGGCCCTGAGTGGGTGTCTAGAATCTCCCCAAACAGCG
GCAACATCGGCTACACCGACAGCGTGAAGGGCAGATTCACCATCAGCAGAG
ATAATGCCAAGAACACCCTGTATCTGCAGATGAACAACCTGAAGCCCGAGG
ACACCGCCCTGTACTACTGCGCCATCGGCCTGAACCTGAGCTCCAGCAGCG
TGCGGGGCCAGGGCACACAGGTTACAGTGTCCAGCGGTACCAAGCTTGGGCC
CGAACAAAAACTCATCTCAGAAGAGGATCTGAATAGCGCCGTCGACCATCATCA
TCATCATCATTGA
SEQ ID NO: 46
ATGGACCCTGCTGAGGCCGTGCTGCAAGAGAAGGCCCTGAAATTCATGAACAGCAGC
GAGCGGGAAGATTGCAACAACGGCGAACCTCCTAGAAAGATCATCCCCGAGAAGAATA
GCCTGAGACAGACCTACAACTCTTGCGCCCGCCTCTGTCTGAACCAGGAGACCGTGT
GCCTGGCTTCCACAGCTATGAAAACCGAGAATTGCGTGGCCAAGACCAAGCTTGCTAA
TGGCACATCTAGCATGATCGTGCCTAAGCAGAGAAAGCTGTCCGCCTCTTACGAGAAG
GAAAAAGAACTGTGCGTCAAGTACTTCGAGCAGTGGAGCGAGTCTGATCAGGTGGAAT
TCGTGGAACACCTGATCAGCCAGATGTGCCACTACCAGCACGGCCACATCAACAGCTA
CCTGAAGCCTATGCTGCAGAGAGACTTCATCACCGCCCTGCCTGCCAGAGGCCTGGA
CCATATCGCCGAGAACATCCTGAGCTACCTGGACGCCAAAAGCCTGTGTGCCGCCGA
GCTGGTGTGCAAGGAGTGGTATAGAGTGACAAGCGATGGAATGCTGTGGAAGAAGCT
GATTGAGAGAATGGTGCGGACCGACAGCCTGTGGCGGGGCCTGGCCGAACGGAGAG
GCTGGGGACAGTACCTGTTTAAGAACAAGCCCCCAGACGGCAACGCCCCTCCAAACA
GCTTCTACAGAGCCCTGTACCCCAAGATCATCCAGGACATCGAAACCATCGAGAGCAA
CTGGAGGTGTGGCCGGCACTCCCTGCAAAGAATCCACTGCAGAGGATCCCAAGTGC
AGCTGGTGGAAACCGGCGGCGGACTCGTGCAGCCTGGCGGATCTCTGCGGC
TGAGCTGTGCCGCTTCTGGCTTTACATTCAGCAGCGTGTACATGAATTGGGT
CAGACAGGCCCCTGGAAAAGGCCCTGAGTGGGTGTCTAGAATCTCCCCAAA
CAGCGGCAACATCGGCTACACCGACAGCGTGAAGGGCAGATTCACCATCAG
CAGAGATAATGCCAAGAACACCCTGTATCTGCAGATGAACAACCTGAAGCC
CGAGGACACCGCCCTGTACTACTGCGCCATCGGCCTGAACCTGAGCTCCAG
CAGCGTGCGGGGCCAGGGCACACAGGTTACAGTGTCCAGC
The sequence bolded above in each of SEQ ID NO: SEQ ID NO: 43 and 45 is SEQ
ID NO: 4, and the sequence bolded in each of SEQ ID NO: 44 and 46 is SEQ ID NO: 47
(shown below).
SEQ ID NO: 47
GGATCCCAAGTGCAGCTGGTGGAAACCGGCGGCGGACTCGTGCAGCCTGGC
GGATCTCTGCGGCTGAGCTGTGCCGCTTCTGGCTTTACATTCAGCAGCGTGT
ACATGAATTGGGTCAGACAGGCCCCTGGAAAAGGCCCTGAGTGGGTGTCTA
GAATCTCCCCAAACAGCGGCAACATCGGCTACACCGACAGCGTGAAGGGCA
GATTCACCATCAGCAGAGATAATGCCAAGAACACCCTGTATCTGCAGATGAA
CAACCTGAAGCCCGAGGACACCGCCCTGTACTACTGCGCCATCGGCCTGAA
CCTGAGCTCCAGCAGCGTGCGGGGCCAGGGCACACAGGTTACAGTGTCCAG
C
The sequence in italics above in each of SEQ ID NOs: 43-46 is SEQ ID NO: 68 as shown
below.
SEQ ID NO: 68
ATGGACCCTGCTGAGGCCGTGCTGCAAGAGAAGGCCCTGAAATTCATGAACAGCAGC
GAGCGGGAAGATTGCAACAACGGCGAACCTCCTAGAAAGATCATCCCCGAGAAGAATA
GCCTGAGACAGACCTACAACTCTTGCGCCCGCCTCTGTCTGAACCAGGAGACCGTGT
GCCTGGCTTCCACAGCTATGAAAACCGAGAATTGCGTGGCCAAGACCAAGCTTGCTAA
TGGCACATCTAGCATGATCGTGCCTAAGCAGAGAAAGCTGTCCGCCTCTTACGAGAAG
GAAAAAGAACTGTGCGTCAAGTACTTCGAGCAGTGGAGCGAGTCTGATCAGGTGGAAT
TCGTGGAACACCTGATCAGCCAGATGTGCCACTACCAGCACGGCCACATCAACAGCTA
CCTGAAGCCTATGCTGCAGAGAGACTTCATCACCGCCCTGCCTGCCAGAGGCCTGGA
CCATATCGCCGAGAACATCCTGAGCTACCTGGACGCCAAAAGCCTGTGTGCCGCCGA
GCTGGTGTGCAAGGAGTGGTATAGAGTGACAAGCGATGGAATGCTGTGGAAGAAGCT
GATTGAGAGAATGGTGCGGACCGACAGCCTGTGGCGGGGCCTGGCCGAACGGAGAG
GCTGGGGACAGTACCTGTTTAAGAACAAGCCCCCAGACGGCAACGCCCCTCCAAACA
GCTTCTACAGAGCCCTGTACCCCAAGATCATCCAGGACATCGAAACCATCGAGAGCAA
CTGGAGGTGTGGCCGGCACTCCCTGCAAAGAATCCACTGCAGA
Beta-Trc-OptTy1 Amino acid sequence (SEQ ID NO: 48)
MDPAEAVLQEKALKFMNSSEREDCNNGEPPRKIIPEKNSLRQTYNSCARLCLNQETVCLAS
TAMKTENCVAKTKLANGTSSMIVPKQRKLSASYEKEKELCVKYFEQWSESDQVEFVEHLIS
QMCHYQHGHINSYLKPMLQRDFITALPARGLDHIAENILSYLDAKSLCAAELVCKEWYRVT
SDGMLWKKLIERMVRTDSLWRGLAERRGWGQYLFKNKPPDGNAPPNSFYRALYPKIIQDI
ETIESNWRCGRHSLQRIHCRQVQLVETGGGLVQPGGSLRLSCAASGFTFSSVYMN
WVRQAPGKGPEWVSRISPNSGNIGYTDSVKGRFTISRDNAKNTLYLQMNNLKPE
DTALYYCAIGLNLSSSSVRGQGTQVTVSS*
or Beta-Trc-OptTy1 Amino acid sequence (SEQ ID NO: 49)
MDPAEAVLQEKALKFMNSSEREDCNNGEPPRKIIPEKNSLRQTYNSCARLCLNQETVCLAS
TAMKTENCVAKTKLANGTSSMIVPKQRKLSASYEKEKELCVKYFEQWSESDQVEFVEHLIS
QMCHYQHGHINSYLKPMLQRDFITALPARGLDHIAENILSYLDAKSLCAAELVCKEWYRVT
SDGMLWKKLIERMVRTDSLWRGLAERRGWGQYLFKNKPPDGNAPPNSFYRALYPKIIQDI
ETIESNWRCGRHSLQRIHCRGSQVQLVETGGGLVQPGGSLRLSCAASGFTFSSVYM
NWVRQAPGKGPEWVSRISPNSGNIGYTDSVKGRFTISRDNAKNTLYLQMNNLK
PEDTALYYCAIGLNLSSSSVRGQGTQVTVSS*
*indicates portion at end of sequence may optionally further SEQ ID NO: 10 as shown in
SEQ ID NOs: 50-51 below.
SEQ ID NO: 50
MDPAEAVLQEKALKFMNSSEREDCNNGEPPRKIIPEKNSLRQTYNSCARLCLNQETVCLAS
TAMKTENCVAKTKLANGTSSMIVPKQRKLSASYEKEKELCVKYFEQWSESDQVEFVEHLIS
QMCHYQHGHINSYLKPMLQRDFITALPARGLDHIAENILSYLDAKSLCAAELVCKEWYRVT
SDGMLWKKLIERMVRTDSLWRGLAERRGWGQYLFKNKPPDGNAPPNSFYRALYPKIIQDI
ETIESNWRCGRHSLQRIHCRQVQLVETGGGLVQPGGSLRLSCAASGFTFSSVYMN
WVRQAPGKGPEWVSRISPNSGNIGYTDSVKGRFTISRDNAKNTLYLQMNNLKPE
DTALYYCAIGLNLSSSSVRGQGTQVTVSSGTKLGPEQKLISEEDLNSAVDHHHHHH
SEQ ID NO: 51
MDPAEAVLQEKALKFMNSSEREDCNNGEPPRKIIPEKNSLRQTYNSCARLCLNQETVCLAS
TAMKTENCVAKTKLANGTSSMIVPKQRKLSASYEKEKELCVKYFEQWSESDQVEFVEHLIS
QMCHYQHGHINSYLKPMLQRDFITALPARGLDHIAENILSYLDAKSLCAAELVCKEWYRVT
SDGMLWKKLIERMVRTDSLWRGLAERRGWGQYLFKNKPPDGNAPPNSFYRALYPKIIQDI
ETIESNWRCGRHSLQRIHCRGSQVQLVETGGGLVQPGGSLRLSCAASGFTFSSVYM
NWVRQAPGKGPEWVSRISPNSGNIGYTDSVKGRFTISRDNAKNTLYLQMNNLK
PEDTALYYCAIGLNLSSSSVRGQGTQVTVSSGTKLGPEQKLISEEDLNSAVDHHHH
HH
The sequence bolded above in each of SEQ ID NO: SEQ ID NO: 48 and 50 is SEQ ID NO:
11, and the sequence bolded in each of SEQ ID NO: 49 and 51 is SEQ ID NO: 67 (shown
below).
SEQ ID NO: 67
GSQVQLVETGGGLVQPGGSLRLSCAASGFTFSSVYMNWVRQAPGKGPEWVSRI
SPNSGNIGYTDSVKGRFTISRDNAKNTLYLQMNNLKPEDTALYYCAIGLNLSSSS
VRGQGTQVTVSS
The sequence in italics above in each of SEQ ID NOs: 48-51 is SEQ ID NO: 69 as shown
below.
SEQ ID NO: 69
MDPAEAVLQEKALKFMNSSEREDCNNGEPPRKIIPEKNSLRQTYNSCARLCLNQETVCLAS
TAMKTENCVAKTKLANGTSSMIVPKQRKLSASYEKEKELCVKYFEQWSESDQVEFVEHLIS
QMCHYQHGHINSYLKPMLQRDFITALPARGLDHIAENILSYLDAKSLCAAELVCKEWYRVT
SDGMLWKKLIERMVRTDSLWRGLAERRGWGQYLFKNKPPDGNAPPNSFYRALYPKIIQDI
ETIESNWRCGRHSLQRIHCRPMLQRDFITALPARGLDHIAENILSYLDAKSLCAAELVCKE
WYRVTSDGMLWKKLIERMVRTDSLWRGLAERRGWGQYLFKNKPPDGNAPPNSFYRALYP
KIIQDIETIESNWRCGRHSLQRIHCR
6. Chimera 6 (Lane 7)
Beta-Trc-OptH11-H4 DNA sequence (SEQ ID NO: 52)
ATGGACCCTGCTGAGGCCGTGCTGCAAGAGAAGGCCCTGAAATTCATGAACAGCAGC
GAGCGGGAAGATTGCAACAACGGCGAACCTCCTAGAAAGATCATCCCCGAGAAGAATA
GCCTGAGACAGACCTACAACTCTTGCGCCCGCCTCTGTCTGAACCAGGAGACCGTGT
GCCTGGCTTCCACAGCTATGAAAACCGAGAATTGCGTGGCCAAGACCAAGCTTGCTAA
TGGCACATCTAGCATGATCGTGCCTAAGCAGAGAAAGCTGTCCGCCTCTTACGAGAAG
GAAAAAGAACTGTGCGTCAAGTACTTCGAGCAGTGGAGCGAGTCTGATCAGGTGGAAT
TCGTGGAACACCTGATCAGCCAGATGTGCCACTACCAGCACGGCCACATCAACAGCTA
CCTGAAGCCTATGCTGCAGAGAGACTTCATCACCGCCCTGCCTGCCAGAGGCCTGGA
CCATATCGCCGAGAACATCCTGAGCTACCTGGACGCCAAAAGCCTGTGTGCCGCCGA
GCTGGTGTGCAAGGAGTGGTATAGAGTGACAAGCGATGGAATGCTGTGGAAGAAGCT
GATTGAGAGAATGGTGCGGACCGACAGCCTGTGGCGGGGCCTGGCCGAACGGAGAG
GCTGGGGACAGTACCTGTTTAAGAACAAGCCCCCAGACGGCAACGCCCCTCCAAACA
GCTTCTACAGAGCCCTGTACCCCAAGATCATCCAGGACATCGAAACCATCGAGAGCAA
CTGGAGGTGTGGCCGGCACTCCCTGCAAAGAATCCACTGCAGACAAGTGCAGCTG
GTGGAAAGCGGCGGAGGCCTGATGCAGGCTGGCGGCAGCCTCAGACTGAG
CTGCGCCGTCAGCGGCCGGACCTTCTCTACAGCCGCCATGGGCTGGTTTAG
ACAGGCCCCTGGCAAGGAACGCGAGTTCGTGGCCGCTATCCGGTGGTCCGG
CGGATCTGCCTACTACGCTGATAGCGTGAAGGGCAGATTCACCATCAGCAG
AGATAAGGCCAAGAACACCGTGTACCTGCAGATGAACAGCCTGAAATACGA
GGACACCGCCGTGTACTATTGTGCCCAGACCCACTACGTGTCCTACCTGCTG
AGCGACTACGCCACCTGGCCCTACGACTACTGGGGACAGGGCACACAGGTT
ACAGTGTCTAGCAAG*
or Beta-Trc-OptH11-H4 DNA sequence (SEQ ID NO: 53)
ATGGACCCTGCTGAGGCCGTGCTGCAAGAGAAGGCCCTGAAATTCATGAACAGCAGC
GAGCGGGAAGATTGCAACAACGGCGAACCTCCTAGAAAGATCATCCCCGAGAAGAATA
GCCTGAGACAGACCTACAACTCTTGCGCCCGCCTCTGTCTGAACCAGGAGACCGTGT
GCCTGGCTTCCACAGCTATGAAAACCGAGAATTGCGTGGCCAAGACCAAGCTTGCTAA
TGGCACATCTAGCATGATCGTGCCTAAGCAGAGAAAGCTGTCCGCCTCTTACGAGAAG
GAAAAAGAACTGTGCGTCAAGTACTTCGAGCAGTGGAGCGAGTCTGATCAGGTGGAAT
TCGTGGAACACCTGATCAGCCAGATGTGCCACTACCAGCACGGCCACATCAACAGCTA
CCTGAAGCCTATGCTGCAGAGAGACTTCATCACCGCCCTGCCTGCCAGAGGCCTGGA
CCATATCGCCGAGAACATCCTGAGCTACCTGGACGCCAAAAGCCTGTGTGCCGCCGA
GCTGGTGTGCAAGGAGTGGTATAGAGTGACAAGCGATGGAATGCTGTGGAAGAAGCT
GATTGAGAGAATGGTGCGGACCGACAGCCTGTGGCGGGGCCTGGCCGAACGGAGAG
GCTGGGGACAGTACCTGTTTAAGAACAAGCCCCCAGACGGCAACGCCCCTCCAAACA
GCTTCTACAGAGCCCTGTACCCCAAGATCATCCAGGACATCGAAACCATCGAGAGCAA
CTGGAGGTGTGGCCGGCACTCCCTGCAAAGAATCCACTGCAGAGGATCCCAAGTGC
AGCTGGTGGAAAGCGGCGGAGGCCTGATGCAGGCTGGCGGCAGCCTCAGA
CTGAGCTGCGCCGTCAGCGGCCGGACCTTCTCTACAGCCGCCATGGGCTGG
TTTAGACAGGCCCCTGGCAAGGAACGCGAGTTCGTGGCCGCTATCCGGTGG
TCCGGCGGATCTGCCTACTACGCTGATAGCGTGAAGGGCAGATTCACCATC
AGCAGAGATAAGGCCAAGAACACCGTGTACCTGCAGATGAACAGCCTGAAA
TACGAGGACACCGCCGTGTACTATTGTGCCCAGACCCACTACGTGTCCTACC
TGCTGAGCGACTACGCCACCTGGCCCTACGACTACTGGGGACAGGGCACAC
AGGTTACAGTGTCTAGCAAG*
*indicates portion at end of sequence may optionally further SEQ ID NO: 3 as shown below
in SEQ ID NOs: 54 and 55).
SEQ ID NO: 54:
ATGGACCCTGCTGAGGCCGTGCTGCAAGAGAAGGCCCTGAAATTCATGAACAGCAGC
GAGCGGGAAGATTGCAACAACGGCGAACCTCCTAGAAAGATCATCCCCGAGAAGAATA
GCCTGAGACAGACCTACAACTCTTGCGCCCGCCTCTGTCTGAACCAGGAGACCGTGT
GCCTGGCTTCCACAGCTATGAAAACCGAGAATTGCGTGGCCAAGACCAAGCTTGCTAA
TGGCACATCTAGCATGATCGTGCCTAAGCAGAGAAAGCTGTCCGCCTCTTACGAGAAG
GAAAAAGAACTGTGCGTCAAGTACTTCGAGCAGTGGAGCGAGTCTGATCAGGTGGAAT
TCGTGGAACACCTGATCAGCCAGATGTGCCACTACCAGCACGGCCACATCAACAGCTA
CCTGAAGCCTATGCTGCAGAGAGACTTCATCACCGCCCTGCCTGCCAGAGGCCTGGA
CCATATCGCCGAGAACATCCTGAGCTACCTGGACGCCAAAAGCCTGTGTGCCGCCGA
GCTGGTGTGCAAGGAGTGGTATAGAGTGACAAGCGATGGAATGCTGTGGAAGAAGCT
GATTGAGAGAATGGTGCGGACCGACAGCCTGTGGCGGGGCCTGGCCGAACGGAGAG
GCTGGGGACAGTACCTGTTTAAGAACAAGCCCCCAGACGGCAACGCCCCTCCAAACA
GCTTCTACAGAGCCCTGTACCCCAAGATCATCCAGGACATCGAAACCATCGAGAGCAA
CTGGAGGTGTGGCCGGCACTCCCTGCAAAGAATCCACTGCAGACAAGTGCAGCTG
GTGGAAAGCGGCGGAGGCCTGATGCAGGCTGGCGGCAGCCTCAGACTGAG
CTGCGCCGTCAGCGGCCGGACCTTCTCTACAGCCGCCATGGGCTGGTTTAG
ACAGGCCCCTGGCAAGGAACGCGAGTTCGTGGCCGCTATCCGGTGGTCCGG
CGGATCTGCCTACTACGCTGATAGCGTGAAGGGCAGATTCACCATCAGCAG
AGATAAGGCCAAGAACACCGTGTACCTGCAGATGAACAGCCTGAAATACGA
GGACACCGCCGTGTACTATTGTGCCCAGACCCACTACGTGTCCTACCTGCTG
AGCGACTACGCCACCTGGCCCTACGACTACTGGGGACAGGGCACACAGGTT
ACAGTGTCTAGCAAGGGTACCAAGCTTGGGCCCGAACAAAAACTCATCTCAGA
AGAGGATCTGAATAGCGCCGTCGACCATCATCATCATCATCATTGA
SEQ ID NO: 55:
ATGGACCCTGCTGAGGCCGTGCTGCAAGAGAAGGCCCTGAAATTCATGAACAGCAGC
GAGCGGGAAGATTGCAACAACGGCGAACCTCCTAGAAAGATCATCCCCGAGAAGAATA
GCCTGAGACAGACCTACAACTCTTGCGCCCGCCTCTGTCTGAACCAGGAGACCGTGT
GCCTGGCTTCCACAGCTATGAAAACCGAGAATTGCGTGGCCAAGACCAAGCTTGCTAA
TGGCACATCTAGCATGATCGTGCCTAAGCAGAGAAAGCTGTCCGCCTCTTACGAGAAG
GAAAAAGAACTGTGCGTCAAGTACTTCGAGCAGTGGAGCGAGTCTGATCAGGTGGAAT
TCGTGGAACACCTGATCAGCCAGATGTGCCACTACCAGCACGGCCACATCAACAGCTA
CCTGAAGCCTATGCTGCAGAGAGACTTCATCACCGCCCTGCCTGCCAGAGGCCTGGA
CCATATCGCCGAGAACATCCTGAGCTACCTGGACGCCAAAAGCCTGTGTGCCGCCGA
GCTGGTGTGCAAGGAGTGGTATAGAGTGACAAGCGATGGAATGCTGTGGAAGAAGCT
GATTGAGAGAATGGTGCGGACCGACAGCCTGTGGCGGGGCCTGGCCGAACGGAGAG
GCTGGGGACAGTACCTGTTTAAGAACAAGCCCCCAGACGGCAACGCCCCTCCAAACA
GCTTCTACAGAGCCCTGTACCCCAAGATCATCCAGGACATCGAAACCATCGAGAGCAA
CTGGAGGTGTGGCCGGCACTCCCTGCAAAGAATCCACTGCAGAGGATCCCAAGTGC
AGCTGGTGGAAAGCGGCGGAGGCCTGATGCAGGCTGGCGGCAGCCTCAGA
CTGAGCTGCGCCGTCAGCGGCCGGACCTTCTCTACAGCCGCCATGGGCTGG
TTTAGACAGGCCCCTGGCAAGGAACGCGAGTTCGTGGCCGCTATCCGGTGG
TCCGGCGGATCTGCCTACTACGCTGATAGCGTGAAGGGCAGATTCACCATC
AGCAGAGATAAGGCCAAGAACACCGTGTACCTGCAGATGAACAGCCTGAAA
TACGAGGACACCGCCGTGTACTATTGTGCCCAGACCCACTACGTGTCCTACC
TGCTGAGCGACTACGCCACCTGGCCCTACGACTACTGGGGACAGGGCACAC
AGGTTACAGTGTCTAGCAAGGGTACCAAGCTTGGGCCCGAACAAAAACTCATC
TCAGAAGAGGATCTGAATAGCGCCGTCGACCATCATCATCATCATCATTGA
The sequence bolded above in each of SEQ ID NOs: 52 and 54 is SEQ ID NO: 17 and
the sequence bolded above in each of SEQ ID NOs: 53 and 55 is SEQ ID NO: 56 (shown
below).
SEQ ID NO: 56
GGATCCCAAGTGCAGCTGGTGGAAAGCGGCGGAGGCCTGATGCAGGCTGG
CGGCAGCCTCAGACTGAGCTGCGCCGTCAGCGGCCGGACCTTCTCTACAGC
CGCCATGGGCTGGTTTAGACAGGCCCCTGGCAAGGAACGCGAGTTCGTGGC
CGCTATCCGGTGGTCCGGCGGATCTGCCTACTACGCTGATAGCGTGAAGGG
CAGATTCACCATCAGCAGAGATAAGGCCAAGAACACCGTGTACCTGCAGAT
GAACAGCCTGAAATACGAGGACACCGCCGTGTACTATTGTGCCCAGACCCA
CTACGTGTCCTACCTGCTGAGCGACTACGCCACCTGGCCCTACGACTACTG
GGGACAGGGCACACAGGTTACAGTGTCTAGCAAG
Beta-Trc-OptH11-H4 Amino acid sequence (SEQ ID NO: 57)
MDPAEAVLQEKALKFMNSSEREDCNNGEPPRKIIPEKNSLRQTYNSCARLCLNQETVCLAS
TAMKTENCVAKTKLANGTSSMIVPKQRKLSASYEKEKELCVKYFEQWSESDQVEFVEHLIS
QMCHYQHGHINSYLKPMLQRDFITALPARGLDHIAENILSYLDAKSLCAAELVCKEWYRVT
SDGMLWKKLIERMVRTDSLWRGLAERRGWGQYLFKNKPPDGNAPPNSFYRALYPKIIQDI
ETIESNWRCGRHSLQRIHCRQVQLVESGGGLMQAGGSLRLSCAVSGRTFSTAAMG
WFRQAPGKEREFVAAIRWSGGSAYYADSVKGRFTISRDKAKNTVYLQMNSLKY
EDTAVYYCAQTHYVSYLLSDYATWPYDYWGQGTQVTVSSK*
or Beta-Trc-OptH11-H4 Amino acid sequence (SEQ ID NO: 58)
MDPAEAVLQEKALKFMNSSEREDCNNGEPPRKIIPEKNSLRQTYNSCARLCLNQETVCLAS
TAMKTENCVAKTKLANGTSSMIVPKQRKLSASYEKEKELCVKYFEQWSESDQVEFVEHLIS
QMCHYQHGHINSYLKPMLQRDFITALPARGLDHIAENILSYLDAKSLCAAELVCKEWYRVT
SDGMLWKKLIERMVRTDSLWRGLAERRGWGQYLFKNKPPDGNAPPNSFYRALYPKIIQDI
ETIESNWRCGRHSLQRIHCRGSQVQLVESGGGLMQAGGSLRLSCAVSGRTFSTAA
MGWFRQAPGKEREFVAAIRWSGGSAYYADSVKGRFTISRDKAKNTVYLQMNS
LKYEDTAVYYCAQTHYVSYLLSDYATWPYDYWGQGTQVTVSSK*
*indicates portion at end of sequence may optionally further SEQ ID NO: 10 as shown
below in SEQ ID NOs: 59 and 60.
SEQ ID NO: 59
MDPAEAVLQEKALKFMNSSEREDCNNGEPPRKIIPEKNSLRQTYNSCARLCLNQETVCLAS
TAMKTENCVAKTKLANGTSSMIVPKQRKLSASYEKEKELCVKYFEQWSESDQVEFVEHLIS
QMCHYQHGHINSYLKPMLQRDFITALPARGLDHIAENILSYLDAKSLCAAELVCKEWYRVT
SDGMLWKKLIERMVRTDSLWRGLAERRGWGQYLFKNKPPDGNAPPNSFYRALYPKIIQDI
ETIESNWRCGRHSLQRIHCRQVQLVESGGGLMQAGGSLRLSCAVSGRTESTAAMG
WFRQAPGKEREFVAAIRWSGGSAYYADSVKGRFTISRDKAKNTVYLQMNSLKY
EDTAVYYCAQTHYVSYLLSDYATWPYDYWGQGTQVTVSSKGTKLGPEQKLISEE
DLNSAVDHHHHHH
SEQ ID NO: 60
MDPAEAVLQEKALKFMNSSEREDCNNGEPPRKIIPEKNSLRQTYNSCARLCLNQETVCLAS
TAMKTENCVAKTKLANGTSSMIVPKQRKLSASYEKEKELCVKYFEQWSESDQVEFVEHLIS
QMCHYQHGHINSYLKPMLQRDFITALPARGLDHIAENILSYLDAKSLCAAELVCKEWYRVT
SDGMLWKKLIERMVRTDSLWRGLAERRGWGQYLFKNKPPDGNAPPNSFYRALYPKIIQDI
ETIESNWRCGRHSLQRIHCRGSQVQLVESGGGLMQAGGSLRLSCAVSGRTFSTAA
MGWFRQAPGKEREFVAAIRWSGGSAYYADSVKGRFTISRDKAKNTVYLQMNS
LKYEDTAVYYCAQTHYVSYLLSDYATWPYDYWGQGTQVTVSSKGTKLGPEQKL
ISEEDLNSAVDHHHHHH
The sequence bolded above in each of SEQ ID NOs: 57 and 59 is SEQ ID NO: 22 and
the sequence bolded above in each of SEQ ID NOs: 58 and 60 is SEQ ID NO: 56 (shown
below).
SEQ ID NO: 70
GSQVQLVESGGGLMQAGGSLRLSCAVSGRTFSTAAMGWFRQAPGKEREFVAAI
RWSGGSAYYADSVKGRFTISRDKAKNTVYLQMNSLKYEDTAVYYCAQTHYVSY
LLSDYATWPYDYWGQGTQVTVSSK
Opt-Spike-Defur DNA sequence (SEQ ID NO: 61)
ATGTTTGTGTTACTCCCACCCGCATATACAAATTCCTTCACCAGAGGAGTG
TACTATCCTGACAAAGTGTTTCGGTCAAGTGTCCTCCACTCTACCTTGGTGTTGCT
TCCACTGGTCAGTTCCCAATGCGTTAATCTCACCACCCGAACTCATCAGGACCTC
TTTCTGCCTTTCTTTTCTAACGTTACATGGTTTCATGCAATCCATGTGTCTGGGAC
AAACGGCACCAAACGCTTCGACAACCCTGTATTGCCATTCAATGATGGGGTGTAC
TTTGCCTCCACAGAGAAATCCAACATCATTCGAGGATGGATTTTCGGGACTACTC
TGGACTCAAAGACACAGAGCCTGCTGATCGTTAACAACGCCACAAACGTTGTCA
TCAAAGTGTGCGAATTCCAGTTTTGCAATGATCCCTTCCTGGGAGTGTACTATCA
CAAGAATAACAAGTCCTGGATGGAGAGCGAATTTCGGGTCTACAGCAGCGCAAA
CAACTGCACCTTCGAGTACGTGAGTCAACCCTTTCTGATGGACCTGGAAGGGAAA
CAGGGAAACTTCAAGAACCTGAGAGAGTTTGTCTTTAAGAACATCGACGGCTATT
TTAAGATCTATAGTAAGCATACGCCTATCAACCTGGTAAGGGATCTTCCCCAGGG
CTTTTCAGCCCTGGAACCTTTGGTTGACTTGCCTATTGGTATCAATATCACCAGAT
TTCAGACCCTTCTGGCATTGCATCGGTCTTATCTTACTCCAGGTGATTCCTCCTCC
GGGTGGACTGCCGGCGCCGCTGCCTACTATGTCGGCTATCTGCAACCAAGAACGT
TCCTGCTCAAGTACAACGAAAACGGCACTATTACGGATGCTGTTGATTGTGCCCT
GGACCCTCTGTCTGAGACTAAATGCACCCTCAAGAGCTTTACCGTTGAGAAGGGG
ATTTACCAAACCAGTAATTTCCGGGTCCAACCCACCGAAAGCATTGTGCGGTTCC
CAAATATCACCAATCTGTGTCCCTTTGGCGAAGTGTTCAATGCTACAAGGTTTGC
TTCTGTGTACGCATGGAATAGGAAACGCATCTCCAATTGTGTCGCTGATTACTCC
GTGCTGTACAATTCCGCCTCTTTCTCAACCTTCAAGTGTTATGGCGTTTCACCTAC
CAAACTTAACGACCTGTGCTTCACTAATGTGTATGCCGACTCTTTTGTGATACGA
GGCGATGAAGTGAGACAGATTGCACCAGGGCAGACCGGCAAAATTGCCGACTAC
AACTACAAGCTTCCAGATGACTTTACCGGATGTGTTATTGCATGGAACTCAAACA
ATCTGGATTCCAAGGTGGGTGGCAACTATAACTACCTGTATAGACTGTTCAGGAA
ATCCAACCTGAAACCATTCGAGCGAGATATAAGCACAGAAATCTACCAGGCTGG
AAGTACGCCCTGCAACGGCGTGGAAGGGTTCAACTGCTACTTCCCATTGCAGAGT
TACGGATTCCAGCCTACAAACGGGGTGGGTTACCAACCCTATCGTGTCGTAGTCC
TGAGTTTTGAGCTCCTCCATGCCCCAGCCACAGTCTGTGGCCCCAAGAAAAGCAC
CAATCTGGTGAAGAACAAATGCGTGAACTTTAACTTTAACGGACTCACAGGAAC
CGGCGTATTGACGGAGAGTAACAAGAAGTTCCTGCCATTCCAGCAGTTCGGTCGC
GATATTGCCGACACTACCGACGCTGTCCGAGATCCCCAGACATTGGAGATTCTTG
ATATCACACCCTGTAGTTTCGGCGGAGTGAGCGTGATTACGCCCGGAACCAATAC
CAGCAATCAGGTTGCCGTCCTGTATCAGGACGTGAATTGCACCGAGGTACCTGTC
GCCATCCACGCTGACCAACTTACACCCACATGGCGAGTATATTCCACCGGCTCCA
ACGTCTTTCAGACACGTGCTGGATGTCTGATCGGTGCAGAACACGTTAATAATAG
CTACGAGTGTGATATCCCCATCGGTGCTGGAATATGCGCCTCTTATCAAACTCAA
ACCAACTCTCCTAGGGCGGCAGCTAGTGTAGCATCCCAAAGTATCATTGCCTACA
CAATGAGCCTCGGTGCTGAGAATTCTGTCGCCTACAGCAACAACTCCATTGCTAT
CCCTACTAACTTCACAATCAGTGTGACAACTGAAATTCTGCCCGTATCTATGACC
AAAACAAGCGTTGACTGCACCATGTACATCTGTGGCGATTCTACCGAATGTAGCA
ATCTCCTCCTGCAATACGGATCATTCTGCACTCAGCTGAATCGTGCCCTCACAGG
TATTGCAGTTGAGCAGGACAAGAATACGCAGGAAGTGTTTGCCCAGGTGAAGCA
AATCTACAAAACTCCACCCATAAAAGACTTTGGCGGATTCAATTTCTCACAGATC
CTGCCCGATCCCTCAAAACCCTCCAAGCGTAGCTTTATCGAGGATCTGCTCTTCA
ACAAGGTAACCCTCGCAGATGCCGGTTTCATCAAGCAGTATGGCGATTGTCTGGG
AGACATCGCCGCTCGGGACCTGATCTGTGCACAGAAGTTCAATGGACTGACCGT
GCTGCCTCCCTTGCTGACCGACGAGATGATAGCCCAATACACTAGCGCCCTGCTG
GCCGGCACCATCACTTCTGGGTGGACATTCGGAGCTGGCGCTGCCCTTCAGATTC
CTTTTGCTATGCAGATGGCCTACCGCTTTAACGGCATCGGTGTGACACAAAACGT
TCTGTATGAAAACCAGAAACTCATCGCCAACCAGTTCAACAGTGCTATCGGTAAG
ATACAGGATAGCCTGTCATCCACTGCCAGCGCATTGGGAAAGTTGCAGGATGTA
GTGAACCAGAATGCCCAGGCACTTAACACCCTGGTGAAACAGCTCTCTTCAAATT
TTGGTGCCATTTCTAGCGTGCTGAATGACATACTGAGCCGGTTGGACAAGGTGGA
GGCTGAAGTGCAGATTGATAGGCTGATAACTGGGCGCCTTCAGTCTCTTCAGACC
TATGTGACCCAGCAGCTCATCCGCGCTGCTGAAATTCGCGCATCCGCTAACCTGG
CAGCAACCAAAATGTCCGAGTGTGTGCTGGGTCAGTCTAAGAGAGTGGACTTTTG
CGGGAAGGGGTATCACCTGATGTCTTTTCCTCAGTCTGCACCCCATGGTGTGGTC
TTTCTGCACGTGACTTATGTCCCAGCTCAGGAAAAGAACTTCACTACAGCCCCAG
CCATCTGCCACGATGGGAAAGCCCACTTTCCCAGGGAAGGCGTATTCGTGTCCAA
TGGTACTCATTGGTTCGTCACTCAGAGAAATTTCTACGAGCCCCAGATTATAACC
ACTGACAATACATTTGTATCCGGCAATTGTGATGTGGTTATCGGGATTGTGAATA
ATACTGTTTACGATCCTTTGCAGCCAGAGCTGGACTCCTTCAAGGAGGAGCTTGA
CAAATATTTTAAGAATCACACATCACCTGACGTCGACCTCGGAGATATTTCAGGA
ATCAATGCTTCCGTGGTCAATATTCAGAAGGAGATAGACAGGCTGAATGAGGTT
GCCAAGAACCTCAACGAGTCTCTGATCGATCTGCAGGAGTTGGGCAAGTACGAA
CAGTATATCAAATGGCCATGGTACATTTGGCTTGGGTTCATTGCTGGGCTGATAG
CTATCGTCATGGTGACAATTATGTTGTGTTGCATGACATCCTGCTGTAGTTGTCTG
AAGGGCTGCTGCTCATGCGGCAGCTGTTGCGACTACAAAGACGATGACGACAAG
TAA
Opt-Spike-Defur Amino acid sequence (SEQ ID NO: 62)
MFVFLVLLPLVSSQCVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDS
KTQSLLIVNNATNVVIKVCEFQFCNDPFLGVYYHKNNKSWMESEFRVYSSANNCTF
EYVSQPFLMDLEGKQGNFKNLREFVFKNIDGYFKIYSKHTPINLVRDLPQGFSALEPL
VDLPIGINITRFQTLLALHRSYLTPGDSSSGWTAGAAAYYVGYLQPRTFLLKYNENGT
ITDAVDCALDPLSETKCTLKSFTVEKGIYQTSNFRVQPTESIVRFPNITNLCPFGEVEN
ATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYLYRLF
RKSNLKPFERDISTEIYQAGSTPCNGVEGENCYFPLQSYGFQPTNGVGYQPYRVVVLS
FELLHAPATVCGPKKSTNLVKNKCVNFNFNGLTGTGVLTESNKKFLPFQQFGRDIAD
TTDAVRDPQTLEILDITPCSFGGVSVITPGTNTSNQVAVLYQDVNCTEVPVAIHADQL
TPTWRVYSTGSNVFQTRAGCLIGAEHVNNSYECDIPIGAGICASYQTQTNSPRAAASV
ASQSIIAYTMSLGAENSVAYSNNSIAIPTNFTISVTTEILPVSMTKTSVDCTMYICGDST
ECSNLLLQYGSFCTQLNRALTGIAVEQDKNTQEVFAQVKQIYKTPPIKDFGGFNFSQI
LPDPSKPSKRSFIEDLLFNKVTLADAGFIKQYGDCLGDIAARDLICAQKFNGLTVLPPL
LTDEMIAQYTSALLAGTITSGWTFGAGAALQIPFAMQMAYRENGIGVTQNVLYENQ
KLIANQFNSAIGKIQDSLSSTASALGKLQDVVNQNAQALNTLVKQLSSNFGAISSVLN
DILSRLDKVEAEVQIDRLITGRLQSLQTYVTQQLIRAAEIRASANLAATKMSECVLGQ
SKRVDFCGKGYHLMSFPQSAPHGVVFLHVTYVPAQEKNFTTAPAICHDGKAHFPRE
GVFVSNGTHWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDLQELGKYEQ
YIKWPWYIWLGFIAGLIAIVMVTIMLCCMTSCCSCLKGCCSCGSCCDYKDDDDK

TABLE 4

| | Spike Specific Nanobody | | Protein Chimera |
|---|---|---|---|
| Lane | Sequence (Ab) | E3 Ligase Sequence (P) | Sequence (Ab-L-P) |
| Lane 2- | OptTy1 DNA Sequence | VHL DNA Sequence | OpTy1-VHL DNA |
| Chimera | (SEQ ID NO: 4 or 63) | (SEQ ID NO: 5) | sequence (SEQ ID |
| 1 | OptTy1 Amino Acid Sequence | VHL Amino Acid Sequence | NO: 1 or SEQ ID |
| | (SEQ ID NO: 11 or 64) | (SEQ ID NO: 12) | NO: 6) |
| | | | OpTy1-VHL Amino |
| | | | Acid Sequence |
| | | | (SEQ ID NO: 8 or |
| | | | SEQ ID NO: 13) |
| Lane 3- | OptH11-H4 DNA Sequence | VHL DNA Sequence | OptH11-H4-VHL |
| Chimera | (SEQ ID NO: 17 or 65) | (SEQ ID NO: 5) | DNA sequence |
| 2 | OptH11-H4 Amino Acid | VHL Amino Acid Sequence | (SEQ ID NO: 15 or |
| | Sequence (SEQ ID NO: 22 or | (SEQ ID NO: 12 | SEQ ID NO: 18) |
| | 66) | | OptH11-H4-VHL |
| | | | Amino Acid |
| | | | sequence (SEQ ID |
| | | | NO: 20 or SEQ ID |
| | | | NO: 23) |
| Lane 4- | OptTy1 DNA Sequence | CHIP DNA sequence | OptTy1-CHIP DNA |
| Chimera | (SEQ ID NO: 4 or 63) | (SEQ ID NO: 27) | sequence (SEQ ID |
| 3 | OptTy1 Amino Acid Sequence | CHIP Amino Acid sequence | NO: 25 or SEQ ID |
| | (SEQ ID NO: 11 or 64) | (SEQ ID NO: 32) | NO: 28) |
| | | | OptTy1-CHIP Amino |
| | | | Acid sequence |
| | | | (SEQ ID NO: 30 or |
| | | | SEQ ID NO: 33) |
| Lane 5- | OptH11-H4 DNA sequence | CHIP DNA sequence | OptH11-H4-CHIP |
| Chimera | (SEQ ID NO: 17 or 65) | (SEQ ID NO: 27) | DNA sequence |
| 4 | OptTy1 Amino Acid Sequence | CHIP Amino Acid sequence | (SEQ ID NO: 35 or |
| | (SEQ ID NO: 22 or 66) | (SEQ ID NO: 32) | SEQ ID NO: 37) |
| | | | OptH11-H4-CHIP |
| | | | Amino Acid |
| | | | sequence (SEQ ID |
| | | | NO: 39 or SEQ ID |
| | | | NO: 41) |
| Lane 6- | OptTy1 DNA sequence | Beta-Trc DNA sequence | Beta-Trc-OptTy1 |
| Chimera | (SEQ ID NO: 4 or 47) | (SEQ ID NO: 68) | DNA sequence |
| 5 | OptTy1 Amino Acid sequence | Beta-Trc Amino Acid sequence | (SEQ ID NO: 43 or |
| | (SEQ ID NO: 11 or 67) | (SEQ ID NO: 69) | SEQ ID NO: 44) |
| | | | Beta-Trc-OptTy1 |
| | | | Amino Acid |
| | | | sequence (SEQ ID |
| | | | NO: 48 or SEQ ID |
| | | | NO: 49 |
| Lane 7- | OptH11-H4 DNA sequence | Beta-Trc DNA sequence | Beta-Trc-OptH11-H4 |
| Chimera | (SEQ ID NO: 17 or 56) | (SEQ ID NO: 68) | DNA sequence |
| 6 | OptH11-H4 Amino Acid | Beta-Trc Amino Acid sequence | (SEQ ID NO: 52 or |
| | sequence | (SEQ ID NO: 69) | SEQ ID NO: 53) |
| | (SEQ ID NO: 22 or 70) | | Beta-Trc-OptH11-H4 |
| | | | Amino Acid |
| | | | sequence (SEQ ID |
| | | | NO: 57 or SEQ ID |
| | | | NO: 58) |
| | Opt-Spike-Defur DNA | | |
| | Sequence (SEQ ID NO: 61) | | |
| | Opt-Spike-Defur Amino Acid | | |
| | sequence (SEQ ID NO: 62) | | |

### Example 2

### SARS-CoV Propagation and Titration

1. Preparation and maintenance of VeroE6 cells (ATCC, CRL-1586).
   a. Quickly thaw VeroE6 cells in a 37° C. water bath.
   b. Centrifuge the cell suspension at 300×g for 6 minutes at 4° C. Discard the supernatant and resuspend the cell pellet in 2 mL of cEMEM.
   c. Transfer the cell suspension to a T25 flask containing 8 mL of cEMEM. Incubate cells at 37° C., 5% CO2, and 95% humidity.
   d. When the cell monolayer reaches 70%-80% confluence, discard the cell culture medium and wash the cell monolayer with 10 mL PBS.
   e. Add 2-3 mL of trypsin and place cells in a 37° C. incubator for 5 minutes. Use a microscope to check whether the cells are detached.
   f. After the cell monolayer is detached, add 10 mL of cEMEM to inactivate trypsin.
   g. Aliquot cells into 4 T175 flasks.
   h. Incubate cells at 37° C. CO₂, and 95% humidity.
2. Preparation of SARS-CoV-2 viral stocks.
   a. When the VeroE6 cell monolayer in the T175 flask reaches 70%-80% confluence, move the flask to a biosafety level 3 containment and place the cells in an incubator at 37° C., 5% CO2, and 95% humidity while preparing the viral inoculum.
   b. Prepare viral inoculum with MOI=0.01 in 5 mL of infection medium.
   c. C. Pour off the medium in the flask and infect the cells by adding the inoculum.
   d. Incubate cells at 37° C., 5% CO2, and 95% humidity for 1 h (gently shake the flask every 15 min).
   e. Add 20 mL of production medium. Incubate the flask at 37° C., 5% CO2, and 95% humidity.
   f. Cells are monitored daily and cytopathic effects are examined under the microscope.
   g. When 50%-70% of the monolayer of cells shows signs of cytopathic effect (approximately day 5-6 post-infection), collect the virus-containing supernatant and aliquot into cryovials.
   h. Store the vials at −80° C.
3. Titration of SARS-CoV-2 viral stocks by PFU quantification.
   a. Prepare VeroE6 cell suspension as described above.
   b. Collect the cells into a 50 mL conical tube and centrifuge at 300×g for 6 min at 4° C. Resuspend the cell pellet in 1 mL of cEMEM. Count cells using trypan blue.
   c. Culture 1.2×10⁵ viable cells per well in 24-well plates. Triplicate wells for uninfected controls, triplicate wells for undiluted viral stocks, and triplicate wells for 10-fold serial dilutions (at least 4 dilutions). In this case, consider at least 18 wells per viral stock.
   d. Incubate the plate at 37° C., 5% CO2, and 95% humidity for 16 h.
   e. Check VeroE6 cell confluency in a 24-well plate (monolayer cells should reach ˜70% confluence).
   f. Move the 24-well plate to a biosafety level 3 laboratory and place in an incubator at 37° C., 5% CO2, 95% humidity while preparing viral inoculum.
   g. thaw 3 aliquots of SARS-CoV-2 virus stock.
   h. Prepare a ten-fold dilution in 1000 μL of infection medium, up to a maximum of 10-7 of the virus stock.
   i. Carefully discard the medium from the 24-well plate containing VeroE6 cells by slow aspiration to avoid cell monolayer detachment.
   j. Infection each well with 200 μL of virus at an appropriate dilution level.
   k. Incubate cells at 37° C., 5% CO2, and 95% humidity for 1 h (manually stir the plate slowly every 15 min).
   l. After incubation, add 800 μL of Overlay medium. Incubate the plate at 37° C., 5% CO2, and 95% humidity for 3 days.
   m. Carefully aspirate and discard the covering medium in each well. Wash the wells once with PBS 1×.
   n. Gently fill the wells with 1 mL of 4% paraformaldehyde (PFA). Incubate at 20° C. (room temperature, RT) for 20-30 min. This step will inactivate the virus.
   o. Aspirate PFA and add 1 mL of PBS for 1×. At this stage, the plate can be removed from the biosafety level 3 containment.
   p. Aspirate PBS and add 300 μL of crystal violet solution to each well. Incubate at 20° C. (RT) for 30 min.
   q. Aspirate crystal violet and discard in a Type IV waste container. Wash the wells 2-4 times with tap water and blot dry.
   r. PFU can be counted using a microscope.

### Adenovirus Propagation and Titration

1. Preparation and maintenance of AD-293 cells (Agilent, 240085).
   a. Quickly thaw AD-293 cells in a 37° C. water bath.
   b. Centrifuge the cell suspension at 300×g for 6 min at 4° C. Discard the supernatant and resuspend the cell pellet in 2 mL of cDMEM.
   c. Transfer the cell suspension to a T25 flask containing 8 mL of cDMEM. Incubate cells at 37° C., 5% CO2, and 95% humidity.
   d. When the cell monolayer reaches 70%-80% confluence, the cell culture medium is discarded and the cell monolayer is washed with 10 mL of PBS.
   e. Add 2-3 mL of trypsin and leave the cells at room temperature for 5 min. Use a microscope to check if the cells are shed.
   f. After cell monolayer isolation, add 10 mL of cDMEM to inactivate trypsin.
   g. Aliquot the cells into 4 T175 flasks.
   h. Incubate cells at 37° C., 5% CO2, and 95% humidity.
2. Preparation of Adenovirus stocks.
   a. When the AD-293 cell monolayer in the T175 flask reaches 70%-80% confluence, place the cells in an incubator at 37° C., 5% CO2, and 95% humidity while preparing the viral inoculum.
   b. Prepare viral inoculum with MOI=0.01 in 5 mL of cDMEM inoculum.
   c. Incubate cells at 37° C., 5% CO2, and 95% humidity for 1 h (gently shake the flask every 15 min).
   d. Add 20 mL of cDMEM. Incubate the flask at 37° C., 5% CO2, and 95% humidity.
   e. Cells are monitored daily and cytopathic effects are examined under the microscope.
   f. After the required number of days of incubation, harvest the cells in the smallest volume of PBS. Prepare the amplified virus stock by 4 rounds of freezing/thawing.
   g. Store the vials at −80° C.
3. Titration of Adenovirus stocks by PFU quantification.
   a. Prepare AD-293 cell suspension as described above.
   b. Collect the cells into a 50 mL conical tube and centrifuge at 300×g for 6 min at 4° C. Resuspend the cell pellet in 1 mL of cDMEM. Count cells using trypan blue.
   c. Culture 1.2×10⁵ viable cells per well in 24-well plates. Triplicate wells for uninfected controls, triplicate wells for undiluted viral stocks, and triplicate wells for 10-fold serial dilutions (at least 7 dilutions). In this case, consider at least 24 wells per viral stock.
   d. Incubate the plate at 37° C., 5% C02, and 95% humidity for 16 h.
   e. Check the confluence of AD293 cells in a 24-well plate (monolayer cells should reach ˜70% confluence).
   f. F. Thaw 3 aliquots of adenovirus stock.
   g. Prepare a ten-fold dilution in 1000 μL of infection medium, up to 10-9 of the virus stock.
   h. Carefully discard the medium from the 24-well plate containing AD-293 cells by slow aspiration to avoid cell monolayer detachment.
   i. Infiltrate each well with 200 μL of virus at an appropriate dilution.
   j. Incubate cells at 37° C., 5% C02, and 95% humidity for 1 h (manually stir the plate slowly every 15 min).
   k. After incubation, add 800 μL of cDMEM. Incubate the plate at 37° C., 5% C02, and 95% humidity for 3 days.
l. PFU can be counted with a microscope.
   k18-hACE2 Housing

Mice are maintained at a 12:12 light cycle, 18° C.-25° C., 45%-65% humidity.

Provide ad libitum drinking and a standardized synthetic diet.

In our study, we will use adult female k18-hACE2 mice (8-12 weeks old).

### Reagent & Resource

### Bacterial and Virus Strains

SARS-CoV-2 alpha variant (B.1.1.7) BEI Resources NR-55461
SARS-CoV-2 delta variant (B.1.617.2) BEI Resources NR-55611
SARS-CoV-2 omicron variant (B.1.1.529.18) BEI Resources NR-56461

### Chemicals, Peptides, and Recombinant Proteins

EMEM high glucose Gibco Cat. #31966-021
DMEM high glucose Gibco Cat. #11965092
Fetal bovine serum, heat inactivated Gibco Cat. #10500-064
Penicillin/streptomycin Gibco Cat. #15140-122
PBS 1× Gibco Cat. #10010015
Trypsin Sigma Cat. #T4049
Carboxymethylcellulose powder Sigma Cat. #C4888
Crystal violet Sigma Cat. #HT9032
Paraformaldehyde 4% Thermo Fisher Cat. #J61984-0500
Trypan blue Sigma Cat. #T8154
Isoflurane (Isovet) B. Braun Cat. #469860
DietGel® Boost ClearH2O Cat. #72-04-5022
HydroGel® ClearH2O Cat. #70-01-5022
Duphalyte® Zoetis Cat #VTMD402045
Lactate ringer B. Braun Cat. #455008
Anima Strath Pencivet Cat. #0250033

### Experimental Models: Cell Lines

VeroE6 ATCC CRL-1586
AD-293 Aligent 240085

### Experimental Models: Organisms/Strains

K18-hACE2 (B6.Cg-Tg(K18-ACE2)2Prlmn/J) transgenic female mice
Jackson Laboratory MSR_JAX:034860

### Materials and Equipment

Complete EMEM (cEMEM): EMEM with 10% of Fetal Bovine Serum (FBS) and 1% of Penicillin/Streptomycin. cEMEM may be stored at 4° C.
Complete DMEM (cDMEM): DMEM with 10% of Fetal Bovine Serum (FBS) and 1% of Penicillin/Streptomycin. cDMEM may be stored at 4° C.
Infection media: EMEM with 1% of Penicillin/Streptomycin. Infection media may be stored at 4° C.
Production media: EMEM high glucose with 2% of FBS and 1% of Penicillin/Streptomycin. Production media may be stored at 4° C.
Carboxymethylcellulose (CMC) 3%: dissolve 3 g in 100 mL of MilliQ-water. Autoclave to sterilize. CMC may be stored at 4° C.
Overlay media: mix CMC 3% solution with EMEM supplemented with 4% of FBS and 2% of Penicillin/Streptomycin media (1:1 proportion). Overlay media may be stored at 4° C.

### Method Details

### Intranasal Inoculation of SARS-CoV-2

8- to 12-week-old mice are infected with SARS-CoV-2 by intranasal administration (nostril tip drip technique). All of the following steps are performed under biosafety level 3 containment by a person with the ability to handle animals under biosafety level 3 conditions.
a. Thaw aliquots of virus stock solution.
b. Prepare the inoculum obtained in the preparation of the SARS-CoV-2 virus stock solution at the required concentration in the infection medium. For the selection of virus concentrations for use in vivo, please refer to the note at the end of this section.
c. Mice are anesthetized by inhalation of 3%-4% isoflurane, induced at 1 L/min of 02 and maintained with 1%-3% oxygen. Mice are anesthetized prior to dosing.
d. An appropriate viral dose is delivered to each nostril of the animal in the amount of 30 μL per animal, up to 15 μL of inoculum. The micropipette is held close to the nose of the mouse and the inoculum is added dropwise. The volume of fluid in each nostril does not exceed ˜15-20 μL. A more precise dose of infection can be achieved with a P20 pipette.
e. The respiration of the mice is observed during the process.
f. The mice are placed back into their cages and monitored for consciousness and normal behavior.
g. The above procedure is repeated for all animals. The inoculum is stored at −80° C. for future titration.

After 3 days, the above process is repeated, but a dose of 1×10⁷ CFU of adenovirus expressing different PROTACs is delivered to each nostril of the animal.

Proper anesthesia of mice is essential for proper intranasal administration of SARS-CoV-2. The pipette tips are not placed inside the animal's nostrils; each drop of inoculum is inhaled before dispensing the next drop; and too deep (<1 breath per second) anesthesia is avoided.

Consideration is given to the selection of doses and variants of SARS-CoV-2 to infect mice, as this greatly affects infection outcomes and animal welfare. A low dose of 1×10⁴ PFU per animal, a medium dose of 5×10⁴ PFU per animal, and a high dose of 1×10⁵ PFU per animal are given. For high doses, plan the number of animals carefully, as severe symptoms are expected and the humane endpoint may have to be applied before the end of the experiment. A minimum of 5 animals are used in each experimental group.

Before each intranasal administration, the inoculum is mixed to ensure that the doses administered are equal.

### Clinical Scores

The animals are monitored and weighed prior to the infection (day 0) and then daily to assess their clinical score. All animals are enrolled in a 6-day study. A detailed clinical score is provided below as infected animals can present a wide range of symptoms.

Animals are monitored and weighed prior to infection (Day 0) and then assessed daily for clinical scores. All animals are enrolled in the 6-day study. A detailed clinical score is provided below, as infected animals can exhibit a wide range of symptoms.

### Scores are Based on General Clinical Signs, Namely:

a. Weight variation: points are given according to the % of weight loss relative to initial weight (at day 0 of infection).
   i. No changes or increased weight: 0 points.
   ii. 1%-6% loss: 1 point.
   iii. 7%-14% loss: 2 points.
   iv. 15%-19% loss: 3 points.
   v. 20%-24% loss: 4 points.
   vi. >25% loss: implement the “humane-end-point” (HEP).
b. Body condition score (BCS):
   i. BCS normal (2-3): 0 points.
   ii. Low BCS (1-1.5): 1 point.
c. Appearance:
   i. Normal: 0 points.
   ii. General lack of grooming or piloerection: 1 point.
   iii. Ocular/Nasal discharge: 2 points.
   iv. Eyes closed: 3 points.
d. Behavior:
   i. Normal: 0 points.
   ii. Minor changes or exaggerated response when provoked: 1 point.
   iii. Less mobile/isolated but alert: 2 points.
   iv. Restless or still and not alert: 3 points.
e. Neurological symptoms:
   i. No alterations: 0 points.
   ii. Tremors: 2 points.
   iii. Ataxia: 2 points.
   iv. Seizures: implement the HEP.
f. Hydration: perform skin pinch test.
   i. Normal—skin returns quickly to original shape: 0 points.
   ii. Dehydrated/abnormal skin pinch test—skin takes additional time to return to its normal position: 3 points.
g. Respiratory movements: observe for signs of respiratory distress.
   i. Normal: 0 points.
   ii. Increased/decreased respiratory frequency: 3 points.
   iii. Severe dyspnea (gasping): implement the HEP.
a. Total clinical score is obtained by adding all points.

Mice are humanely euthanized upon reaching the HEP. A clinical score of 15 or higher is considered for the implementation of the HEP of euthanasia because it reflects a significant impairment in the animal's general health and indicates the presence of an irreversible severe disease outcome. Independently of the total score obtained:
a. A weight loss higher than 25%, seizures and severe dyspnea are established HEP.
b. Unresponsiveness to stimulus for 3 sequential observations are established HEP.

For decision orientation, four clinical score intervals are considered:
a. 0-5 points represent minor changes:
   i. Re-evaluate animal the next day.
b. 6-10 points represent moderate changes:
   i. Seek veterinary opinion.
   ii. Re-evaluate after 4 h.
   iii. If persistent after 2 sequential evaluations, consider hydration and food support or HEP application if one of the scores corresponds to a 3 at behavioral changes (unresponsive to stimulus).
c. 11-14 points represent major changes:
   i. Seek veterinary opinion.
   ii. Re-evaluate the animal at a 1 h interval for a maximum of 3 observations.
   iii. Consider hydration, food support and temperature control.
   iv. If unchanged apply a HEP.
d. >15 points represent critical changes:
   i. Apply HEP at a maximum period of 1 h.

To validate and quantify the viral infection, tissues are retrieved and processed in order to perform qPCR absolute quantification (using either a quantified virus stock or a single copy plasmid fragment as a standard curve), immunohistochemistry and/or plaque assays.

Read-outs of the infection progression include the collection of bronchoalveolar lavage, blood, organs (specially lungs and brain due to the tropism of SARS-CoV-2). Specific analyses include the immunological profile by flow cytometry, qPCR or immunoassay, and tissue pathology by histologic analysis and morphometric quantification.

What is claimed is:

1. A chimera molecule having the formula:
Ab-L-P,
wherein Ab is an antibody that specifically binds to a Severe Acute Respiratory Syndrome coronavirus 2 (SARS-CoV-2) Spike (S) protein antigen, the Ab is conjugated to L; L is a linker or a bond covalently or non-covalent bound to Ab and to P; and P is a ubiquitin E3 ligase.

2. The chimera molecule of claim 1, wherein the Ab is a nanobody that specifically binds to the SARS-CoV-2 spike antigen and P is an E2 recruiting domain of the ubiquitin E3 ligase.

3. The chimera molecule of claim 1, wherein the Spike (S) protein antigen is selected from the group consisting of Ty1 and H11-H4.

4. The chimera molecule of claim 1, wherein Ab has an amino acid sequence selected from any one of SEQ ID NOs: 11, 22, 64, 66, 67, or 70, or an amino acid sequence having at least 85% sequence identity to the amino acid sequence of any one of SEQ ID NOs: 11, 22, 64, 66, 67, or 70.

5. The chimera molecule of claim 4, wherein Ab has an amino acid sequence encoded by a nucleic acid sequence selected from any one of SEQ ID NOs: 4, 17, 47, 56, 63, or 65, or an amino acid sequence encoded by a nucleic acid sequence having at least 85% sequence identity to the nucleic acid sequence of any one of SEQ ID NOs: 4, 17, 47, 56, 63, or 65.

6. The chimera molecule of claim 1, wherein P has an amino acid sequence selected from any one of SEQ ID NOs: 12, 32, or 69, or an amino acid sequence having at least 85% sequence identity to the amino acid sequence of any one of SEQ ID NOs: 12, 32, or 69.

7. The chimera molecule of claim 6, wherein P has an amino acid sequence encoded by a nucleic acid sequence selected from any one of 5, 27, or 68, or an amino acid sequence encoded by a nucleic acid sequence having at least 85% sequence identity to the nucleic acid sequence of any one of SEQ ID NOs: 5, 27, or 68.

8. The chimera molecule of claim 1, wherein Ab has an amino acid sequence selected from any one of SEQ ID NOs: 11, 22, 64, 66, 67, or 70, and P has an amino acid sequence selected from any one of SEQ ID NOs: 12, 32, or 69.

9. The chimera molecule of claim 8, wherein Ab has an amino acid sequence encoded by a nucleic acid sequence selected from any one of SEQ ID NOs: 4, 17, 47, 56, 63, or 65, and P has an amino acid sequence encoded by a nucleic acid sequence selected from any one of 5, 27, or 68.

10. The chimera molecule of claim 1, wherein the chimera molecule is a polypeptide or a protein chimera.

11. The chimera molecule of claim 10, wherein the polypeptide or protein chimera has an amino acid sequence selected from any one of SEQ ID NOs: 8, 13, 20, 23, 30, 33, 39, 41, 48, 49, 57, or 58, or an amino acid sequence having at least 85% sequence identity to the amino acid sequence of any one of SEQ ID NOs: 8, 13, 20, 23, 30, 33, 39, 41, 48, 49, 57, or 58.

12. The chimera molecule of claim 11, wherein the polypeptide or protein chimera has an amino acid sequence encoded by a nucleic acid sequence selected from any one of SEQ ID NOs: 1, 6, 5, 18, 25, 28, 35, 37, 43, 44, 52, or 53, or an amino acid sequence encoded by a nucleic acid sequence having at least 85% sequence identity to the nucleic acid sequence of any one of SEQ ID NOs:1, 6, 5, 18, 25, 28, 35, 37, 43, 44, 52, or 53.

13. A composition comprising the chimera molecule of claim 1, and at least one pharmaceutically acceptable excipient.

14. A vaccine comprising the composition of claim 13.

15. A method of treating or preventing a viral infection in a subject in need thereof, comprising administering a therapeutically effective amount of the composition of claim 13, to the subject in need thereof.

16. A method of preventing, reducing severity, reducing incidence, or reducing transmissibility of a SARS-CoV-2 infection in a subject in need thereof, comprising administering a therapeutically effective amount of the composition of claim 13, to the subject in need thereof.

17. A kit or article of manufacture comprising the chimera molecule of claim 1, and a package insert or label indicating that the chimera molecule can be used to treat or prevent a SARS-CoV-2 infection, to reduce severity a SARS-CoV-2 infection, to reduce incidence a SARS-CoV-2 infection, and/or to reduce transmissibility of a SARS-CoV-2 infection.


SEQUENCE LISTING (70 sequences)

SEQ ID NO: 1 - DNA, 546 bp, synthetic construct
  source 1..546: mol_type = other DNA
caagtgcagctggtggaaaccggcggcggactcgtgcagcctggcggatctctgcggctgagctgtgccgcttctggctttacattcagcagcgtgtacatgaattgggtcagacaggcccctggaaaaggccctgagtgggtgtctagaatctccccaaacagcggcaacatcggctacaccgacagcgtgaagggcagattcaccatcagcagagataatgccaagaacaccctgtatctgcagatgaacaacctgaagcccgaggacaccgccctgtactactgcgccatcggcctgaacctgagctccagcagcgtgcggggccagggcacacaggttacagtgtccagcggatccacactgcctgtgtacaccctgaaagagcggtgcctgcaagtggtcagatctctcgtgaagcctgagaactacagaagactggacatcgtgcggagcctgtacgaggatctggaagatcaccccaacgtgcagaaggacctggaacggctgacccaggagagaatcgcccaccagagaatgggcgac

SEQ ID NO: 2 - DNA, 630 bp, synthetic construct
  source 1..630: mol_type = other DNA
caagtgcagctggtggaaaccggcggcggactcgtgcagcctggcggatctctgcggctgagctgtgccgcttctggctttacattcagcagcgtgtacatgaattgggtcagacaggcccctggaaaaggccctgagtgggtgtctagaatctccccaaacagcggcaacatcggctacaccgacagcgtgaagggcagattcaccatcagcagagataatgccaagaacaccctgtatctgcagatgaacaacctgaagcccgaggacaccgccctgtactactgcgccatcggcctgaacctgagctccagcagcgtgcggggccagggcacacaggttacagtgtccagcggatccacactgcctgtgtacaccctgaaagagcggtgcctgcaagtggtcagatctctcgtgaagcctgagaactacagaagactggacatcgtgcggagcctgtacgaggatctggaagatcaccccaacgtgcagaaggacctggaacggctgacccaggagagaatcgcccaccagagaatgggcgacggtaccaagcttgggcccgaacaaaaactcatctcagaagaggatctgaatagcgccgtcgaccatcatcatcatcatcattga

SEQ ID NO: 3 - DNA, 84 bp, synthetic construct
  source 1..84: mol_type = other DNA
ggtaccaagcttgggcccgaacaaaaactcatctcagaagaggatctgaatagcgccgtcgaccatcatcatcatcatcattga

SEQ ID NO: 4 - DNA, 354 bp, synthetic construct
  source 1..354: mol_type = other DNA
caagtgcagctggtggaaaccggcggcggactcgtgcagcctggcggatctctgcggctgagctgtgccgcttctggctttacattcagcagcgtgtacatgaattgggtcagacaggcccctggaaaaggccctgagtgggtgtctagaatctccccaaacagcggcaacatcggctacaccgacagcgtgaagggcagattcaccatcagcagagataatgccaagaacaccctgtatctgcagatgaacaacctgaagcccgaggacaccgccctgtactactgcgccatcggcctgaacctgagctccagcagcgtgcggggccagggcacacaggttacagtgtccagc

SEQ ID NO: 5 - DNA, 192 bp, synthetic construct
  source 1..192: mol_type = other DNA
ggatccacactgcctgtgtacaccctgaaagagcggtgcctgcaagtggtcagatctctcgtgaagcctgagaactacagaagactggacatcgtgcggagcctgtacgaggatctggaagatcaccccaacgtgcagaaggacctggaacggctgacccaggagagaatcgcccaccagagaatgggcgac

SEQ ID NO: 6 - DNA, 549 bp, synthetic construct
  source 1..549: mol_type = other DNA
atgcaagtgcagctggtggaaaccggcggcggactcgtgcagcctggcggatctctgcggctgagctgtgccgcttctggctttacattcagcagcgtgtacatgaattgggtcagacaggcccctggaaaaggccctgagtgggtgtctagaatctccccaaacagcggcaacatcggctacaccgacagcgtgaagggcagattcaccatcagcagagataatgccaagaacaccctgtatctgcagatgaacaacctgaagcccgaggacaccgccctgtactactgcgccatcggcctgaacctgagctccagcagcgtgcggggccagggcacacaggttacagtgtccagcggatccacactgcctgtgtacaccctgaaagagcggtgcctgcaagtggtcagatctctcgtgaagcctgagaactacagaagactggacatcgtgcggagcctgtacgaggatctggaagatcaccccaacgtgcagaaggacctggaacggctgacccaggagagaatcgcccaccagagaatgggcgac

SEQ ID NO: 7 - DNA, 633 bp, synthetic construct
  source 1..633: mol_type = other DNA
atgcaagtgcagctggtggaaaccggcggcggactcgtgcagcctggcggatctctgcggctgagctgtgccgcttctggctttacattcagcagcgtgtacatgaattgggtcagacaggcccctggaaaaggccctgagtgggtgtctagaatctccccaaacagcggcaacatcggctacaccgacagcgtgaagggcagattcaccatcagcagagataatgccaagaacaccctgtatctgcagatgaacaacctgaagcccgaggacaccgccctgtactactgcgccatcggcctgaacctgagctccagcagcgtgcggggccagggcacacaggttacagtgtccagcggatccacactgcctgtgtacaccctgaaagagcggtgcctgcaagtggtcagatctctcgtgaagcctgagaactacagaagactggacatcgtgcggagcctgtacgaggatctggaagatcaccccaacgtgcagaaggacctggaacggctgacccaggagagaatcgcccaccagagaatgggcgacggtaccaagcttgggcccgaacaaaaactcatctcagaagaggatctgaatagcgccgtcgaccatcatcatcatcatcattga

SEQ ID NO: 8 - protein, 182 aa, synthetic construct
QVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSSGSTLPVYTLKERCLQVVRSLVKPENYRRLDIVRSLYEDLEDHPNVQKDLERLTQERIAHQRMGD

SEQ ID NO: 9 - protein, 209 aa, synthetic construct
QVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSSGSTLPVYTLKERCLQVVRSLVKPENYRRLDIVRSLYEDLEDHPNVQKDLERLTQERIAHQRMGDGTKLGPEQKLISEEDLNSAVDHHHHHH

SEQ ID NO: 10 - protein, 27 aa, synthetic construct
GTKLGPEQKLISEEDLNSAVDHHHHHH

SEQ ID NO: 11 - protein, 118 aa, synthetic construct
QVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSS

SEQ ID NO: 12 - protein, 64 aa, synthetic construct
GSTLPVYTLKERCLQVVRSLVKPENYRRLDIVRSLYEDLEDHPNVQKDLERLTQERIAHQRMGD

SEQ ID NO: 13 - protein, 183 aa, synthetic construct
MQVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSSGSTLPVYTLKERCLQVVRSLVKPENYRRLDIVRSLYEDLEDHPNVQKDLERLTQERIAHQRMGD

SEQ ID NO: 14 - protein, 210 aa, synthetic construct
MQVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSSGSTLPVYTLKERCLQVVRSLVKPENYRRLDIVRSLYEDLEDHPNVQKDLERLTQERIAHQRMGDGTKLGPEQKLISEEDLNSAVDHHHHHH

SEQ ID NO: 15 - DNA, 576 bp, synthetic construct
  source 1..576: mol_type = other DNA
caagtgcagctggtggaaagcggcggaggcctgatgcaggctggcggcagcctcagactgagctgcgccgtcagcggccggaccttctctacagccgccatgggctggtttagacaggcccctggcaaggaacgcgagttcgtggccgctatccggtggtccggcggatctgcctactacgctgatagcgtgaagggcagattcaccatcagcagagataaggccaagaacaccgtgtacctgcagatgaacagcctgaaatacgaggacaccgccgtgtactattgtgcccagacccactacgtgtcctacctgctgagcgactacgccacctggccctacgactactggggacagggcacacaggttacagtgtctagcaagggatccacactgcctgtgtacaccctgaaagagcggtgcctgcaagtggtcagatctctcgtgaagcctgagaactacagaagactggacatcgtgcggagcctgtacgaggatctggaagatcaccccaacgtgcagaaggacctggaacggctgacccaggagagaatcgcccaccagagaatgggcgac

SEQ ID NO: 16 - DNA, 660 bp, synthetic construct
  source 1..660: mol_type = other DNA
caagtgcagctggtggaaagcggcggaggcctgatgcaggctggcggcagcctcagactgagctgcgccgtcagcggccggaccttctctacagccgccatgggctggtttagacaggcccctggcaaggaacgcgagttcgtggccgctatccggtggtccggcggatctgcctactacgctgatagcgtgaagggcagattcaccatcagcagagataaggccaagaacaccgtgtacctgcagatgaacagcctgaaatacgaggacaccgccgtgtactattgtgcccagacccactacgtgtcctacctgctgagcgactacgccacctggccctacgactactggggacagggcacacaggttacagtgtctagcaagggatccacactgcctgtgtacaccctgaaagagcggtgcctgcaagtggtcagatctctcgtgaagcctgagaactacagaagactggacatcgtgcggagcctgtacgaggatctggaagatcaccccaacgtgcagaaggacctggaacggctgacccaggagagaatcgcccaccagagaatgggcgacggtaccaagcttgggcccgaacaaaaactcatctcagaagaggatctgaatagcgccgtcgaccatcatcatcatcatcattga

SEQ ID NO: 17 - DNA, 384 bp, synthetic construct
  source 1..384: mol_type = other DNA
caagtgcagctggtggaaagcggcggaggcctgatgcaggctggcggcagcctcagactgagctgcgccgtcagcggccggaccttctctacagccgccatgggctggtttagacaggcccctggcaaggaacgcgagttcgtggccgctatccggtggtccggcggatctgcctactacgctgatagcgtgaagggcagattcaccatcagcagagataaggccaagaacaccgtgtacctgcagatgaacagcctgaaatacgaggacaccgccgtgtactattgtgcccagacccactacgtgtcctacctgctgagcgactacgccacctggccctacgactactggggacagggcacacaggttacagtgtctagcaag

SEQ ID NO: 18 - DNA, 579 bp, synthetic construct
  source 1..579: mol_type = other DNA
atgcaagtgcagctggtggaaagcggcggaggcctgatgcaggctggcggcagcctcagactgagctgcgccgtcagcggccggaccttctctacagccgccatgggctggtttagacaggcccctggcaaggaacgcgagttcgtggccgctatccggtggtccggcggatctgcctactacgctgatagcgtgaagggcagattcaccatcagcagagataaggccaagaacaccgtgtacctgcagatgaacagcctgaaatacgaggacaccgccgtgtactattgtgcccagacccactacgtgtcctacctgctgagcgactacgccacctggccctacgactactggggacagggcacacaggttacagtgtctagcaagggatccacactgcctgtgtacaccctgaaagagcggtgcctgcaagtggtcagatctctcgtgaagcctgagaactacagaagactggacatcgtgcggagcctgtacgaggatctggaagatcaccccaacgtgcagaaggacctggaacggctgacccaggagagaatcgcccaccagagaatgggcgac

SEQ ID NO: 19 - DNA, 663 bp, synthetic construct
  source 1..663: mol_type = other DNA
atgcaagtgcagctggtggaaagcggcggaggcctgatgcaggctggcggcagcctcagactgagctgcgccgtcagcggccggaccttctctacagccgccatgggctggtttagacaggcccctggcaaggaacgcgagttcgtggccgctatccggtggtccggcggatctgcctactacgctgatagcgtgaagggcagattcaccatcagcagagataaggccaagaacaccgtgtacctgcagatgaacagcctgaaatacgaggacaccgccgtgtactattgtgcccagacccactacgtgtcctacctgctgagcgactacgccacctggccctacgactactggggacagggcacacaggttacagtgtctagcaagggatccacactgcctgtgtacaccctgaaagagcggtgcctgcaagtggtcagatctctcgtgaagcctgagaactacagaagactggacatcgtgcggagcctgtacgaggatctggaagatcaccccaacgtgcagaaggacctggaacggctgacccaggagagaatcgcccaccagagaatgggcgacggtaccaagcttgggcccgaacaaaaactcatctcagaagaggatctgaatagcgccgtcgaccatcatcatcatcatcattga

SEQ ID NO: 20 - protein, 192 aa, synthetic construct
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTHYVSYLLSDYATWPYDYWGQGTQVTVSSKGSTLPVYTLKERCLQVVRSLVKPENYRRLDIVRSLYEDLEDHPNVQKDLERLTQERIAHQRMGD

SEQ ID NO: 21 - protein, 219 aa, synthetic construct
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTHYVSYLLSDYATWPYDYWGQGTQVTVSSKGSTLPVYTLKERCLQVVRSLVKPENYRRLDIVRSLYEDLEDHPNVQKDLERLTQERIAHQRMGDGTKLGPEQKLISEEDLNSAVDHHHHHH

SEQ ID NO: 22 - protein, 128 aa, synthetic construct
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTHYVSYLLSDYATWPYDYWGQGTQVTVSSK

SEQ ID NO: 23 - protein, 193 aa, synthetic construct
MQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTHYVSYLLSDYATWPYDYWGQGTQVTVSSKGSTLPVYTLKERCLQVVRSLVKPENYRRLDIVRSLYEDLEDHPNVQKDLERLTQERIAHQRMGD

SEQ ID NO: 24 - protein, 220 aa, synthetic construct
MQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTHYVSYLLSDYATWPYDYWGQGTQVTVSSKGSTLPVYTLKERCLQVVRSLVKPENYRRLDIVRSLYEDLEDHPNVQKDLERLTQERIAHQRMGDGTKLGPEQKLISEEDLNSAVDHHHHHH

SEQ ID NO: 25 - DNA, 888 bp, synthetic construct
  source 1..888: mol_type = other DNA
caagtgcagctggtggaaaccggcggcggactcgtgcagcctggcggatctctgcggctgagctgtgccgcttctggctttacattcagcagcgtgtacatgaattgggtcagacaggcccctggaaaaggccctgagtgggtgtctagaatctccccaaacagcggcaacatcggctacaccgacagcgtgaagggcagattcaccatcagcagagataatgccaagaacaccctgtatctgcagatgaacaacctgaagcccgaggacaccgccctgtactactgcgccatcggcctgaacctgagctccagcagcgtgcggggccagggcacacaggttacagtgtccagcgaattccgcctcaacttcggcgatgatattccatctgccctgagaatcgctaagaagaaaagatggaacagcatcgaggaaaggcggatccaccaggagagcgagctgcacagctacctgagcagactgatcgccgctgaacgggaaagagaactggaagagtgccagagaaaccacgagggcgacgaggacgacagccacgtgcgggcccagcaggcctgcatcgaggccaagcacgataagtacatggccgacatggacgaactgttcagccaggtcgacgagaagcggaagaagcgggacatccctgattatctgtgcggcaagatctcttttgagctgatgagagagccttgtatcaccccttccggcatcacctacgacagaaaggacatcgaggaacacctgcaaagagtgggacatttcgaccccgtgacaagaagccctctgacccaggagcagctgatccccaatctggccatgaaagaggtgatcgacgccttcatctccgagaacggctgggtggaagattac

SEQ ID NO: 26 - DNA, 972 bp, synthetic construct
  source 1..972: mol_type = other DNA
caagtgcagctggtggaaaccggcggcggactcgtgcagcctggcggatctctgcggctgagctgtgccgcttctggctttacattcagcagcgtgtacatgaattgggtcagacaggcccctggaaaaggccctgagtgggtgtctagaatctccccaaacagcggcaacatcggctacaccgacagcgtgaagggcagattcaccatcagcagagataatgccaagaacaccctgtatctgcagatgaacaacctgaagcccgaggacaccgccctgtactactgcgccatcggcctgaacctgagctccagcagcgtgcggggccagggcacacaggttacagtgtccagcgaattccgcctcaacttcggcgatgatattccatctgccctgagaatcgctaagaagaaaagatggaacagcatcgaggaaaggcggatccaccaggagagcgagctgcacagctacctgagcagactgatcgccgctgaacgggaaagagaactggaagagtgccagagaaaccacgagggcgacgaggacgacagccacgtgcgggcccagcaggcctgcatcgaggccaagcacgataagtacatggccgacatggacgaactgttcagccaggtcgacgagaagcggaagaagcgggacatccctgattatctgtgcggcaagatctcttttgagctgatgagagagccttgtatcaccccttccggcatcacctacgacagaaaggacatcgaggaacacctgcaaagagtgggacatttcgaccccgtgacaagaagccctctgacccaggagcagctgatccccaatctggccatgaaagaggtgatcgacgccttcatctccgagaacggctgggtggaagattacggtaccaagcttgggcccgaacaaaaactcatctcagaagaggatctgaatagcgccgtcgaccatcatcatcatcatcattga

SEQ ID NO: 27 - DNA, 534 bp, synthetic construct
  source 1..534: mol_type = other DNA
gaattccgcctcaacttcggcgatgatattccatctgccctgagaatcgctaagaagaaaagatggaacagcatcgaggaaaggcggatccaccaggagagcgagctgcacagctacctgagcagactgatcgccgctgaacgggaaagagaactggaagagtgccagagaaaccacgagggcgacgaggacgacagccacgtgcgggcccagcaggcctgcatcgaggccaagcacgataagtacatggccgacatggacgaactgttcagccaggtcgacgagaagcggaagaagcgggacatccctgattatctgtgcggcaagatctcttttgagctgatgagagagccttgtatcaccccttccggcatcacctacgacagaaaggacatcgaggaacacctgcaaagagtgggacatttcgaccccgtgacaagaagccctctgacccaggagcagctgatccccaatctggccatgaaagaggtgatcgacgccttcatctccgagaacggctgggtggaagattac

SEQ ID NO: 28 - DNA, 891 bp, synthetic construct
  source 1..891: mol_type = other DNA
atgcaagtgcagctggtggaaaccggcggcggactcgtgcagcctggcggatctctgcggctgagctgtgccgcttctggctttacattcagcagcgtgtacatgaattgggtcagacaggcccctggaaaaggccctgagtgggtgtctagaatctccccaaacagcggcaacatcggctacaccgacagcgtgaagggcagattcaccatcagcagagataatgccaagaacaccctgtatctgcagatgaacaacctgaagcccgaggacaccgccctgtactactgcgccatcggcctgaacctgagctccagcagcgtgcggggccagggcacacaggttacagtgtccagcgaattccgcctcaacttcggcgatgatattccatctgccctgagaatcgctaagaagaaaagatggaacagcatcgaggaaaggcggatccaccaggagagcgagctgcacagctacctgagcagactgatcgccgctgaacgggaaagagaactggaagagtgccagagaaaccacgagggcgacgaggacgacagccacgtgcgggcccagcaggcctgcatcgaggccaagcacgataagtacatggccgacatggacgaactgttcagccaggtcgacgagaagcggaagaagcgggacatccctgattatctgtgcggcaagatctcttttgagctgatgagagagccttgtatcaccccttccggcatcacctacgacagaaaggacatcgaggaacacctgcaaagagtgggacatttcgaccccgtgacaagaagccctctgacccaggagcagctgatccccaatctggccatgaaagaggtgatcgacgccttcatctccgagaacggctgggtggaagattac

SEQ ID NO: 29 - DNA, 975 bp, synthetic construct
  source 1..975: mol_type = other DNA
atgcaagtgcagctggtggaaaccggcggcggactcgtgcagcctggcggatctctgcggctgagctgtgccgcttctggctttacattcagcagcgtgtacatgaattgggtcagacaggcccctggaaaaggccctgagtgggtgtctagaatctccccaaacagcggcaacatcggctacaccgacagcgtgaagggcagattcaccatcagcagagataatgccaagaacaccctgtatctgcagatgaacaacctgaagcccgaggacaccgccctgtactactgcgccatcggcctgaacctgagctccagcagcgtgcggggccagggcacacaggttacagtgtccagcgaattccgcctcaacttcggcgatgatattccatctgccctgagaatcgctaagaagaaaagatggaacagcatcgaggaaaggcggatccaccaggagagcgagctgcacagctacctgagcagactgatcgccgctgaacgggaaagagaactggaagagtgccagagaaaccacgagggcgacgaggacgacagccacgtgcgggcccagcaggcctgcatcgaggccaagcacgataagtacatggccgacatggacgaactgttcagccaggtcgacgagaagcggaagaagcgggacatccctgattatctgtgcggcaagatctcttttgagctgatgagagagccttgtatcaccccttccggcatcacctacgacagaaaggacatcgaggaacacctgcaaagagtgggacatttcgaccccgtgacaagaagccctctgacccaggagcagctgatccccaatctggccatgaaagaggtgatcgacgccttcatctccgagaacggctgggtggaagattacggtaccaagcttgggcccgaacaaaaactcatctcagaagaggatctgaatagcgccgtcgaccatcatcatcatcatcattga

SEQ ID NO: 30 - protein, 296 aa, synthetic construct
QVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSSEFRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 31 - protein, 323 aa, synthetic construct
QVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSSEFRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDYGTKLGPEQKLISEEDLNSAVDHHHHHH

SEQ ID NO: 32 - protein, 178 aa, synthetic construct
EFRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 33 - protein, 297 aa, synthetic construct
MQVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSSEFRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 34 - protein, 324 aa, synthetic construct
MQVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSSEFRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDYGTKLGPEQKLISEEDLNSAVDHHHHHH

SEQ ID NO: 35 - DNA, 918 bp, synthetic construct
  source 1..918: mol_type = other DNA
caagtgcagctggtggaaagcggcggaggcctgatgcaggctggcggcagcctcagactgagctgcgccgtcagcggccggaccttctctacagccgccatgggctggtttagacaggcccctggcaaggaacgcgagttcgtggccgctatccggtggtccggcggatctgcctactacgctgatagcgtgaagggcagattcaccatcagcagagataaggccaagaacaccgtgtacctgcagatgaacagcctgaaatacgaggacaccgccgtgtactattgtgcccagacccactacgtgtcctacctgctgagcgactacgccacctggccctacgactactggggacagggcacacaggttacagtgtctagcaaggaattccgcctcaacttcggcgatgatattccatctgccctgagaatcgctaagaagaaaagatggaacagcatcgaggaaaggcggatccaccaggagagcgagctgcacagctacctgagcagactgatcgccgctgaacgggaaagagaactggaagagtgccagagaaaccacgagggcgacgaggacgacagccacgtgcgggcccagcaggcctgcatcgaggccaagcacgataagtacatggccgacatggacgaactgttcagccaggtcgacgagaagcggaagaagcgggacatccctgattatctgtgcggcaagatctcttttgagctgatgagagagccttgtatcaccccttccggcatcacctacgacagaaaggacatcgaggaacacctgcaaagagtgggacatttcgaccccgtgacaagaagccctctgacccaggagcagctgatccccaatctggccatgaaagaggtgatcgacgccttcatctccgagaacggctgggtggaagattac

SEQ ID NO: 36 - DNA, 1002 bp, synthetic construct
  source 1..1002: mol_type = other DNA
caagtgcagctggtggaaagcggcggaggcctgatgcaggctggcggcagcctcagactgagctgcgccgtcagcggccggaccttctctacagccgccatgggctggtttagacaggcccctggcaaggaacgcgagttcgtggccgctatccggtggtccggcggatctgcctactacgctgatagcgtgaagggcagattcaccatcagcagagataaggccaagaacaccgtgtacctgcagatgaacagcctgaaatacgaggacaccgccgtgtactattgtgcccagacccactacgtgtcctacctgctgagcgactacgccacctggccctacgactactggggacagggcacacaggttacagtgtctagcaaggaattccgcctcaacttcggcgatgatattccatctgccctgagaatcgctaagaagaaaagatggaacagcatcgaggaaaggcggatccaccaggagagcgagctgcacagctacctgagcagactgatcgccgctgaacgggaaagagaactggaagagtgccagagaaaccacgagggcgacgaggacgacagccacgtgcgggcccagcaggcctgcatcgaggccaagcacgataagtacatggccgacatggacgaactgttcagccaggtcgacgagaagcggaagaagcgggacatccctgattatctgtgcggcaagatctcttttgagctgatgagagagccttgtatcaccccttccggcatcacctacgacagaaaggacatcgaggaacacctgcaaagagtgggacatttcgaccccgtgacaagaagccctctgacccaggagcagctgatccccaatctggccatgaaagaggtgatcgacgccttcatctccgagaacggctgggtggaagattacggtaccaagcttgggcccgaacaaaaactcatctcagaagaggatctgaatagcgccgtcgaccatcatcatcatcatcattga

SEQ ID NO: 37 - DNA, 921 bp, synthetic construct
  source 1..921: mol_type = other DNA
atgcaagtgcagctggtggaaagcggcggaggcctgatgcaggctggcggcagcctcagactgagctgcgccgtcagcggccggaccttctctacagccgccatgggctggtttagacaggcccctggcaaggaacgcgagttcgtggccgctatccggtggtccggcggatctgcctactacgctgatagcgtgaagggcagattcaccatcagcagagataaggccaagaacaccgtgtacctgcagatgaacagcctgaaatacgaggacaccgccgtgtactattgtgcccagacccactacgtgtcctacctgctgagcgactacgccacctggccctacgactactggggacagggcacacaggttacagtgtctagcaaggaattccgcctcaacttcggcgatgatattccatctgccctgagaatcgctaagaagaaaagatggaacagcatcgaggaaaggcggatccaccaggagagcgagctgcacagctacctgagcagactgatcgccgctgaacgggaaagagaactggaagagtgccagagaaaccacgagggcgacgaggacgacagccacgtgcgggcccagcaggcctgcatcgaggccaagcacgataagtacatggccgacatggacgaactgttcagccaggtcgacgagaagcggaagaagcgggacatccctgattatctgtgcggcaagatctcttttgagctgatgagagagccttgtatcaccccttccggcatcacctacgacagaaaggacatcgaggaacacctgcaaagagtgggacatttcgaccccgtgacaagaagccctctgacccaggagcagctgatccccaatctggccatgaaagaggtgatcgacgccttcatctccgagaacggctgggtggaagattac

SEQ ID NO: 38 - DNA, 1005 bp, synthetic construct
  source 1..1005: mol_type = other DNA
atgcaagtgcagctggtggaaagcggcggaggcctgatgcaggctggcggcagcctcagactgagctgcgccgtcagcggccggaccttctctacagccgccatgggctggtttagacaggcccctggcaaggaacgcgagttcgtggccgctatccggtggtccggcggatctgcctactacgctgatagcgtgaagggcagattcaccatcagcagagataaggccaagaacaccgtgtacctgcagatgaacagcctgaaatacgaggacaccgccgtgtactattgtgcccagacccactacgtgtcctacctgctgagcgactacgccacctggccctacgactactggggacagggcacacaggttacagtgtctagcaaggaattccgcctcaacttcggcgatgatattccatctgccctgagaatcgctaagaagaaaagatggaacagcatcgaggaaaggcggatccaccaggagagcgagctgcacagctacctgagcagactgatcgccgctgaacgggaaagagaactggaagagtgccagagaaaccacgagggcgacgaggacgacagccacgtgcgggcccagcaggcctgcatcgaggccaagcacgataagtacatggccgacatggacgaactgttcagccaggtcgacgagaagcggaagaagcgggacatccctgattatctgtgcggcaagatctcttttgagctgatgagagagccttgtatcaccccttccggcatcacctacgacagaaaggacatcgaggaacacctgcaaagagtgggacatttcgaccccgtgacaagaagccctctgacccaggagcagctgatccccaatctggccatgaaagaggtgatcgacgccttcatctccgagaacggctgggtggaagattacggtaccaagcttgggcccgaacaaaaactcatctcagaagaggatctgaatagcgccgtcgaccatcatcatcatcatcattga

SEQ ID NO: 39 - protein, 306 aa, synthetic construct
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTHYVSYLLSDYATWPYDYWGQGTQVTVSSKEFRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 40 - protein, 333 aa, synthetic construct
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTHYVSYLLSDYATWPYDYWGQGTQVTVSSKEFRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDYGTKLGPEQKLISEEDLNSAVDHHHHHH

SEQ ID NO: 41 - protein, 307 aa, synthetic construct
MQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTHYVSYLLSDYATWPYDYWGQGTQVTVSSKEFRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 42 - protein, 334 aa, synthetic construct
MQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTHYVSYLLSDYATWPYDYWGQGTQVTVSSKEFRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDYGTKLGPEQKLISEEDLNSAVDHHHHHH

SEQ ID NO: 43 - DNA, 1143 bp, synthetic construct
  source 1..1143: mol_type = other DNA
atggaccctgctgaggccgtgctgcaagagaaggccctgaaattcatgaacagcagcgagcgggaagattgcaacaacggcgaacctcctagaaagatcatccccgagaagaatagcctgagacagacctacaactcttgcgcccgcctctgtctgaaccaggagaccgtgtgcctggcttccacagctatgaaaaccgagaattgcgtggccaagaccaagcttgctaatggcacatctagcatgatcgtgcctaagcagagaaagctgtccgcctcttacgagaaggaaaaagaactgtgcgtcaagtacttcgagcagtggagcgagtctgatcaggtggaattcgtggaacacctgatcagccagatgtgccactaccagcacggccacatcaacagctacctgaagcctatgctgcagagagacttcatcaccgccctgcctgccagaggcctggaccatatcgccgagaacatcctgagctacctggacgccaaaagcctgtgtgccgccgagctggtgtgcaaggagtggtatagagtgacaagcgatggaatgctgtggaagaagctgattgagagaatggtgcggaccgacagcctgtggcggggcctggccgaacggagaggctggggacagtacctgtttaagaacaagcccccagacggcaacgcccctccaaacagcttctacagagccctgtaccccaagatcatccaggacatcgaaaccatcgagagcaactggaggtgtggccggcactccctgcaaagaatccactgcagacaagtgcagctggtggaaaccggcggcggactcgtgcagcctggcggatctctgcggctgagctgtgccgcttctggctttacattcagcagcgtgtacatgaattgggtcagacaggcccctggaaaaggccctgagtgggtgtctagaatctccccaaacagcggcaacatcggctacaccgacagcgtgaagggcagattcaccatcagcagagataatgccaagaacaccctgtatctgcagatgaacaacctgaagcccgaggacaccgccctgtactactgcgccatcggcctgaacctgagctccagcagcgtgcggggccagggcacacaggttacagtgtccagc

SEQ ID NO: 44 - DNA, 1149 bp, synthetic construct
  source 1..1149: mol_type = other DNA
atggaccctgctgaggccgtgctgcaagagaaggccctgaaattcatgaacagcagcgagcgggaagattgcaacaacggcgaacctcctagaaagatcatccccgagaagaatagcctgagacagacctacaactcttgcgcccgcctctgtctgaaccaggagaccgtgtgcctggcttccacagctatgaaaaccgagaattgcgtggccaagaccaagcttgctaatggcacatctagcatgatcgtgcctaagcagagaaagctgtccgcctcttacgagaaggaaaaagaactgtgcgtcaagtacttcgagcagtggagcgagtctgatcaggtggaattcgtggaacacctgatcagccagatgtgccactaccagcacggccacatcaacagctacctgaagcctatgctgcagagagacttcatcaccgccctgcctgccagaggcctggaccatatcgccgagaacatcctgagctacctggacgccaaaagcctgtgtgccgccgagctggtgtgcaaggagtggtatagagtgacaagcgatggaatgctgtggaagaagctgattgagagaatggtgcggaccgacagcctgtggcggggcctggccgaacggagaggctggggacagtacctgtttaagaacaagcccccagacggcaacgcccctccaaacagcttctacagagccctgtaccccaagatcatccaggacatcgaaaccatcgagagcaactggaggtgtggccggcactccctgcaaagaatccactgcagaggatcccaagtgcagctggtggaaaccggcggcggactcgtgcagcctggcggatctctgcggctgagctgtgccgcttctggctttacattcagcagcgtgtacatgaattgggtcagacaggcccctggaaaaggccctgagtgggtgtctagaatctccccaaacagcggcaacatcggctacaccgacagcgtgaagggcagattcaccatcagcagagataatgccaagaacaccctgtatctgcagatgaacaacctgaagcccgaggacaccgccctgtactactgcgccatcggcctgaacctgagctccagcagcgtgcggggccagggcacacaggttacagtgtccagc

SEQ ID NO: 45 - DNA, 1227 bp, synthetic construct
  source 1..1227: mol_type = other DNA
atggaccctgctgaggccgtgctgcaagagaaggccctgaaattcatgaacagcagcgagcgggaagattgcaacaacggcgaacctcctagaaagatcatccccgagaagaatagcctgagacagacctacaactcttgcgcccgcctctgtctgaaccaggagaccgtgtgcctggcttccacagctatgaaaaccgagaattgcgtggccaagaccaagcttgctaatggcacatctagcatgatcgtgcctaagcagagaaagctgtccgcctcttacgagaaggaaaaagaactgtgcgtcaagtacttcgagcagtggagcgagtctgatcaggtggaattcgtggaacacctgatcagccagatgtgccactaccagcacggccacatcaacagctacctgaagcctatgctgcagagagacttcatcaccgccctgcctgccagaggcctggaccatatcgccgagaacatcctgagctacctggacgccaaaagcctgtgtgccgccgagctggtgtgcaaggagtggtatagagtgacaagcgatggaatgctgtggaagaagctgattgagagaatggtgcggaccgacagcctgtggcggggcctggccgaacggagaggctggggacagtacctgtttaagaacaagcccccagacggcaacgcccctccaaacagcttctacagagccctgtaccccaagatcatccaggacatcgaaaccatcgagagcaactggaggtgtggccggcactccctgcaaagaatccactgcagacaagtgcagctggtggaaaccggcggcggactcgtgcagcctggcggatctctgcggctgagctgtgccgcttctggctttacattcagcagcgtgtacatgaattgggtcagacaggcccctggaaaaggccctgagtgggtgtctagaatctccccaaacagcggcaacatcggctacaccgacagcgtgaagggcagattcaccatcagcagagataatgccaagaacaccctgtatctgcagatgaacaacctgaagcccgaggacaccgccctgtactactgcgccatcggcctgaacctgagctccagcagcgtgcggggccagggcacacaggttacagtgtccagcggtaccaagcttgggcccgaacaaaaactcatctcagaagaggatctgaatagcgccgtcgaccatcatcatcatcatcattga

SEQ ID NO: 46 - DNA, 1233 bp, synthetic construct
  source 1..1233: mol_type = other DNA
atggaccctgctgaggccgtgctgcaagagaaggccctgaaattcatgaacagcagcgagcgggaagattgcaacaacggcgaacctcctagaaagatcatccccgagaagaatagcctgagacagacctacaactcttgcgcccgcctctgtctgaaccaggagaccgtgtgcctggcttccacagctatgaaaaccgagaattgcgtggccaagaccaagcttgctaatggcacatctagcatgatcgtgcctaagcagagaaagctgtccgcctcttacgagaaggaaaaagaactgtgcgtcaagtacttcgagcagtggagcgagtctgatcaggtggaattcgtggaacacctgatcagccagatgtgccactaccagcacggccacatcaacagctacctgaagcctatgctgcagagagacttcatcaccgccctgcctgccagaggcctggaccatatcgccgagaacatcctgagctacctggacgccaaaagcctgtgtgccgccgagctggtgtgcaaggagtggtatagagtgacaagcgatggaatgctgtggaagaagctgattgagagaatggtgcggaccgacagcctgtggcggggcctggccgaacggagaggctggggacagtacctgtttaagaacaagcccccagacggcaacgcccctccaaacagcttctacagagccctgtaccccaagatcatccaggacatcgaaaccatcgagagcaactggaggtgtggccggcactccctgcaaagaatccactgcagaggatcccaagtgcagctggtggaaaccggcggcggactcgtgcagcctggcggatctctgcggctgagctgtgccgcttctggctttacattcagcagcgtgtacatgaattgggtcagacaggcccctggaaaaggccctgagtgggtgtctagaatctccccaaacagcggcaacatcggctacaccgacagcgtgaagggcagattcaccatcagcagagataatgccaagaacaccctgtatctgcagatgaacaacctgaagcccgaggacaccgccctgtactactgcgccatcggcctgaacctgagctccagcagcgtgcggggccagggcacacaggttacagtgtccagcggtaccaagcttgggcccgaacaaaaactcatctcagaagaggatctgaatagcgccgtcgaccatcatcatcatcatcattga

SEQ ID NO: 47 - DNA, 360 bp, synthetic construct
  source 1..360: mol_type = other DNA
ggatcccaagtgcagctggtggaaaccggcggcggactcgtgcagcctggcggatctctgcggctgagctgtgccgcttctggctttacattcagcagcgtgtacatgaattgggtcagacaggcccctggaaaaggccctgagtgggtgtctagaatctccccaaacagcggcaacatcggctacaccgacagcgtgaagggcagattcaccatcagcagagataatgccaagaacaccctgtatctgcagatgaacaacctgaagcccgaggacaccgccctgtactactgcgccatcggcctgaacctgagctccagcagcgtgcggggccagggcacacaggttacagtgtccagc

SEQ ID NO: 48 - protein, 381 aa, synthetic construct
MDPAEAVLQEKALKFMNSSEREDCNNGEPPRKIIPEKNSLRQTYNSCARLCLNQETVCLASTAMKTENCVAKTKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLQRIHCRQVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSS

SEQ ID NO: 49 - protein, 383 aa, synthetic construct
MDPAEAVLQEKALKFMNSSEREDCNNGEPPRKIIPEKNSLRQTYNSCARLCLNQETVCLASTAMKTENCVAKTKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLQRIHCRGSQVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSS

SEQ ID NO: 50 - protein, 408 aa, synthetic construct
MDPAEAVLQEKALKFMNSSEREDCNNGEPPRKIIPEKNSLRQTYNSCARLCLNQETVCLASTAMKTENCVAKTKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLQRIHCRQVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSSGTKLGPEQKLISEEDLNSAVDHHHHHH

SEQ ID NO: 51 - protein, 410 aa, synthetic construct
MDPAEAVLQEKALKFMNSSEREDCNNGEPPRKIIPEKNSLRQTYNSCARLCLNQETVCLASTAMKTENCVAKTKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLQRIHCRGSQVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSSGTKLGPEQKLISEEDLNSAVDHHHHHH

SEQ ID NO: 52 - DNA, 1173 bp, synthetic construct
  source 1..1173: mol_type = other DNA
atggaccctgctgaggccgtgctgcaagagaaggccctgaaattcatgaacagcagcgagcgggaagattgcaacaacggcgaacctcctagaaagatcatccccgagaagaatagcctgagacagacctacaactcttgcgcccgcctctgtctgaaccaggagaccgtgtgcctggcttccacagctatgaaaaccgagaattgcgtggccaagaccaagcttgctaatggcacatctagcatgatcgtgcctaagcagagaaagctgtccgcctcttacgagaaggaaaaagaactgtgcgtcaagtacttcgagcagtggagcgagtctgatcaggtggaattcgtggaacacctgatcagccagatgtgccactaccagcacggccacatcaacagctacctgaagcctatgctgcagagagacttcatcaccgccctgcctgccagaggcctggaccatatcgccgagaacatcctgagctacctggacgccaaaagcctgtgtgccgccgagctggtgtgcaaggagtggtatagagtgacaagcgatggaatgctgtggaagaagctgattgagagaatggtgcggaccgacagcctgtggcggggcctggccgaacggagaggctggggacagtacctgtttaagaacaagcccccagacggcaacgcccctccaaacagcttctacagagccctgtaccccaagatcatccaggacatcgaaaccatcgagagcaactggaggtgtggccggcactccctgcaaagaatccactgcagacaagtgcagctggtggaaagcggcggaggcctgatgcaggctggcggcagcctcagactgagctgcgccgtcagcggccggaccttctctacagccgccatgggctggtttagacaggcccctggcaaggaacgcgagttcgtggccgctatccggtggtccggcggatctgcctactacgctgatagcgtgaagggcagattcaccatcagcagagataaggccaagaacaccgtgtacctgcagatgaacagcctgaaatacgaggacaccgccgtgtactattgtgcccagacccactacgtgtcctacctgctgagcgactacgccacctggccctacgactactggggacagggcacacaggttacagtgtctagcaag

SEQ ID NO: 53 - DNA, 1179 bp, synthetic construct
  source 1..1179: mol_type = other DNA
atggaccctgctgaggccgtgctgcaagagaaggccctgaaattcatgaacagcagcgagcgggaagattgcaacaacggcgaacctcctagaaagatcatccccgagaagaatagcctgagacagacctacaactcttgcgcccgcctctgtctgaaccaggagaccgtgtgcctggcttccacagctatgaaaaccgagaattgcgtggccaagaccaagcttgctaatggcacatctagcatgatcgtgcctaagcagagaaagctgtccgcctcttacgagaaggaaaaagaactgtgcgtcaagtacttcgagcagtggagcgagtctgatcaggtggaattcgtggaacacctgatcagccagatgtgccactaccagcacggccacatcaacagctacctgaagcctatgctgcagagagacttcatcaccgccctgcctgccagaggcctggaccatatcgccgagaacatcctgagctacctggacgccaaaagcctgtgtgccgccgagctggtgtgcaaggagtggtatagagtgacaagcgatggaatgctgtggaagaagctgattgagagaatggtgcggaccgacagcctgtggcggggcctggccgaacggagaggctggggacagtacctgtttaagaacaagcccccagacggcaacgcccctccaaacagcttctacagagccctgtaccccaagatcatccaggacatcgaaaccatcgagagcaactggaggtgtggccggcactccctgcaaagaatccactgcagaggatcccaagtgcagctggtggaaagcggcggaggcctgatgcaggctggcggcagcctcagactgagctgcgccgtcagcggccggaccttctctacagccgccatgggctggtttagacaggcccctggcaaggaacgcgagttcgtggccgctatccggtggtccggcggatctgcctactacgctgatagcgtgaagggcagattcaccatcagcagagataaggccaagaacaccgtgtacctgcagatgaacagcctgaaatacgaggacaccgccgtgtactattgtgcccagacccactacgtgtcctacctgctgagcgactacgccacctggccctacgactactggggacagggcacacaggttacagtgtctagcaag

SEQ ID NO: 54 - DNA, 1257 bp, synthetic construct
  source 1..1257: mol_type = other DNA
atggaccctgctgaggccgtgctgcaagagaaggccctgaaattcatgaacagcagcgagcgggaagattgcaacaacggcgaacctcctagaaagatcatccccgagaagaatagcctgagacagacctacaactcttgcgcccgcctctgtctgaaccaggagaccgtgtgcctggcttccacagctatgaaaaccgagaattgcgtggccaagaccaagcttgctaatggcacatctagcatgatcgtgcctaagcagagaaagctgtccgcctcttacgagaaggaaaaagaactgtgcgtcaagtacttcgagcagtggagcgagtctgatcaggtggaattcgtggaacacctgatcagccagatgtgccactaccagcacggccacatcaacagctacctgaagcctatgctgcagagagacttcatcaccgccctgcctgccagaggcctggaccatatcgccgagaacatcctgagctacctggacgccaaaagcctgtgtgccgccgagctggtgtgcaaggagtggtatagagtgacaagcgatggaatgctgtggaagaagctgattgagagaatggtgcggaccgacagcctgtggcggggcctggccgaacggagaggctggggacagtacctgtttaagaacaagcccccagacggcaacgcccctccaaacagcttctacagagccctgtaccccaagatcatccaggacatcgaaaccatcgagagcaactggaggtgtggccggcactccctgcaaagaatccactgcagacaagtgcagctggtggaaagcggcggaggcctgatgcaggctggcggcagcctcagactgagctgcgccgtcagcggccggaccttctctacagccgccatgggctggtttagacaggcccctggcaaggaacgcgagttcgtggccgctatccggtggtccggcggatctgcctactacgctgatagcgtgaagggcagattcaccatcagcagagataaggccaagaacaccgtgtacctgcagatgaacagcctgaaatacgaggacaccgccgtgtactattgtgcccagacccactacgtgtcctacctgctgagcgactacgccacctggccctacgactactggggacagggcacacaggttacagtgtctagcaagggtaccaagcttgggcccgaacaaaaactcatctcagaagaggatctgaatagcgccgtcgaccatcatcatcatcatcattga

SEQ ID NO: 55 - DNA, 1263 bp, synthetic construct
  source 1..1263: mol_type = other DNA
atggaccctgctgaggccgtgctgcaagagaaggccctgaaattcatgaacagcagcgagcgggaagattgcaacaacggcgaacctcctagaaagatcatccccgagaagaatagcctgagacagacctacaactcttgcgcccgcctctgtctgaaccaggagaccgtgtgcctggcttccacagctatgaaaaccgagaattgcgtggccaagaccaagcttgctaatggcacatctagcatgatcgtgcctaagcagagaaagctgtccgcctcttacgagaaggaaaaagaactgtgcgtcaagtacttcgagcagtggagcgagtctgatcaggtggaattcgtggaacacctgatcagccagatgtgccactaccagcacggccacatcaacagctacctgaagcctatgctgcagagagacttcatcaccgccctgcctgccagaggcctggaccatatcgccgagaacatcctgagctacctggacgccaaaagcctgtgtgccgccgagctggtgtgcaaggagtggtatagagtgacaagcgatggaatgctgtggaagaagctgattgagagaatggtgcggaccgacagcctgtggcggggcctggccgaacggagaggctggggacagtacctgtttaagaacaagcccccagacggcaacgcccctccaaacagcttctacagagccctgtaccccaagatcatccaggacatcgaaaccatcgagagcaactggaggtgtggccggcactccctgcaaagaatccactgcagaggatcccaagtgcagctggtggaaagcggcggaggcctgatgcaggctggcggcagcctcagactgagctgcgccgtcagcggccggaccttctctacagccgccatgggctggtttagacaggcccctggcaaggaacgcgagttcgtggccgctatccggtggtccggcggatctgcctactacgctgatagcgtgaagggcagattcaccatcagcagagataaggccaagaacaccgtgtacctgcagatgaacagcctgaaatacgaggacaccgccgtgtactattgtgcccagacccactacgtgtcctacctgctgagcgactacgccacctggccctacgactactggggacagggcacacaggttacagtgtctagcaagggtaccaagcttgggcccgaacaaaaactcatctcagaagaggatctgaatagcgccgtcgaccatcatcatcatcatcattga

SEQ ID NO: 56 - DNA, 390 bp, synthetic construct
  source 1..390: mol_type = other DNA
ggatcccaagtgcagctggtggaaagcggcggaggcctgatgcaggctggcggcagcctcagactgagctgcgccgtcagcggccggaccttctctacagccgccatgggctggtttagacaggcccctggcaaggaacgcgagttcgtggccgctatccggtggtccggcggatctgcctactacgctgatagcgtgaagggcagattcaccatcagcagagataaggccaagaacaccgtgtacctgcagatgaacagcctgaaatacgaggacaccgccgtgtactattgtgcccagacccactacgtgtcctacctgctgagcgactacgccacctggccctacgactactggggacagggcacacaggttacagtgtctagcaag

SEQ ID NO: 57 - protein, 391 aa, synthetic construct
MDPAEAVLQEKALKFMNSSEREDCNNGEPPRKIIPEKNSLRQTYNSCARLCLNQETVCLASTAMKTENCVAKTKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLQRIHCRQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTHYVSYLLSDYATWPYDYWGQGTQVTVSSK

SEQ ID NO: 58 - protein, 393 aa, synthetic construct
MDPAEAVLQEKALKFMNSSEREDCNNGEPPRKIIPEKNSLRQTYNSCARLCLNQETVCLASTAMKTENCVAKTKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLQRIHCRGSQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTHYVSYLLSDYATWPYDYWGQGTQVTVSSK

SEQ ID NO: 59 - protein, 418 aa, synthetic construct
MDPAEAVLQEKALKFMNSSEREDCNNGEPPRKIIPEKNSLRQTYNSCARLCLNQETVCLASTAMKTENCVAKTKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLQRIHCRQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTHYVSYLLSDYATWPYDYWGQGTQVTVSSKGTKLGPEQKLISEEDLNSAVDHHHHHH

SEQ ID NO: 60 - protein, 420 aa, synthetic construct
MDPAEAVLQEKALKFMNSSEREDCNNGEPPRKIIPEKNSLRQTYNSCARLCLNQETVCLASTAMKTENCVAKTKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLQRIHCRGSQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTHYVSYLLSDYATWPYDYWGQGTQVTVSSKGTKLGPEQKLISEEDLNSAVDHHHHHH

SEQ ID NO: 61 - DNA, 3789 bp, synthetic construct
  source 1..3789: mol_type = other DNA
atgtttgtgttactcccacccgcatatacaaattccttcaccagaggagtgtactatcctgacaaagtgtttcggtcaagtgtcctccactctaccttggtgttgcttccactggtcagttcccaatgcgttaatctcaccacccgaactcatcaggacctctttctgcctttcttttctaacgttacatggtttcatgcaatccatgtgtctgggacaaacggcaccaaacgcttcgacaaccctgtattgccattcaatgatggggtgtactttgcctccacagagaaatccaacatcattcgaggatggattttcgggactactctggactcaaagacacagagcctgctgatcgttaacaacgccacaaacgttgtcatcaaagtgtgcgaattccagttttgcaatgatcccttcctgggagtgtactatcacaagaataacaagtcctggatggagagcgaatttcgggtctacagcagcgcaaacaactgcaccttcgagtacgtgagtcaaccctttctgatggacctggaagggaaacagggaaacttcaagaacctgagagagtttgtctttaagaacatcgacggctattttaagatctatagtaagcatacgcctatcaacctggtaagggatcttccccagggcttttcagccctggaacctttggttgacttgcctattggtatcaatatcaccagatttcagacccttctggcattgcatcggtcttatcttactccaggtgattcctcctccgggtggactgccggcgccgctgcctactatgtcggctatctgcaaccaagaacgttcctgctcaagtacaacgaaaacggcactattacggatgctgttgattgtgccctggaccctctgtctgagactaaatgcaccctcaagagctttaccgttgagaaggggatttaccaaaccagtaatttccgggtccaacccaccgaaagcattgtgcggttcccaaatatcaccaatctgtgtccctttggcgaagtgttcaatgctacaaggtttgcttctgtgtacgcatggaataggaaacgcatctccaattgtgtcgctgattactccgtgctgtacaattccgcctctttctcaaccttcaagtgttatggcgtttcacctaccaaacttaacgacctgtgcttcactaatgtgtatgccgactcttttgtgatacgaggcgatgaagtgagacagattgcaccagggcagaccggcaaaattgccgactacaactacaagcttccagatgactttaccggatgtgttattgcatggaactcaaacaatctggattccaaggtgggtggcaactataactacctgtatagactgttcaggaaatccaacctgaaaccattcgagcgagatataagcacagaaatctaccaggctggaagtacgccctgcaacggcgtggaagggttcaactgctacttcccattgcagagttacggattccagcctacaaacggggtgggttaccaaccctatcgtgtcgtagtcctgagttttgagctcctccatgccccagccacagtctgtggccccaagaaaagcaccaatctggtgaagaacaaatgcgtgaactttaactttaacggactcacaggaaccggcgtattgacggagagtaacaagaagttcctgccattccagcagttcggtcgcgatattgccgacactaccgacgctgtccgagatccccagacattggagattcttgatatcacaccctgtagtttcggcggagtgagcgtgattacgcccggaaccaataccagcaatcaggttgccgtcctgtatcaggacgtgaattgcaccgaggtacctgtcgccatccacgctgaccaacttacacccacatggcgagtatattccaccggctccaacgtctttcagacacgtgctggatgtctgatcggtgcagaacacgttaataatagctacgagtgtgatatccccatcggtgctggaatatgcgcctcttatcaaactcaaaccaactctcctagggcggcagctagtgtagcatcccaaagtatcattgcctacacaatgagcctcggtgctgagaattctgtcgcctacagcaacaactccattgctatccctactaacttcacaatcagtgtgacaactgaaattctgcccgtatctatgaccaaaacaagcgttgactgcaccatgtacatctgtggcgattctaccgaatgtagcaatctcctcctgcaatacggatcattctgcactcagctgaatcgtgccctcacaggtattgcagttgagcaggacaagaatacgcaggaagtgtttgcccaggtgaagcaaatctacaaaactccacccataaaagactttggcggattcaatttctcacagatcctgcccgatccctcaaaaccctccaagcgtagctttatcgaggatctgctcttcaacaaggtaaccctcgcagatgccggtttcatcaagcagtatggcgattgtctgggagacatcgccgctcgggacctgatctgtgcacagaagttcaatggactgaccgtgctgcctcccttgctgaccgacgagatgatagcccaatacactagcgccctgctggccggcaccatcacttctgggtggacattcggagctggcgctgcccttcagattccttttgctatgcagatggcctaccgctttaacggcatcggtgtgacacaaaacgttctgtatgaaaaccagaaactcatcgccaaccagttcaacagtgctatcggtaagatacaggatagcctgtcatccactgccagcgcattgggaaagttgcaggatgtagtgaaccagaatgcccaggcacttaacaccctggtgaaacagctctcttcaaattttggtgccatttctagcgtgctgaatgacatactgagccggttggacaaggtggaggctgaagtgcagattgataggctgataactgggcgccttcagtctcttcagacctatgtgacccagcagctcatccgcgctgctgaaattcgcgcatccgctaacctggcagcaaccaaaatgtccgagtgtgtgctgggtcagtctaagagagtggacttttgcgggaaggggtatcacctgatgtcttttcctcagtctgcaccccatggtgtggtctttctgcacgtgacttatgtcccagctcaggaaaagaacttcactacagccccagccatctgccacgatgggaaagcccactttcccagggaaggcgtattcgtgtccaatggtactcattggttcgtcactcagagaaatttctacgagccccagattataaccactgacaatacatttgtatccggcaattgtgatgtggttatcgggattgtgaataatactgtttacgatcctttgcagccagagctggactccttcaaggaggagcttgacaaatattttaagaatcacacatcacctgacgtcgacctcggagatatttcaggaatcaatgcttccgtggtcaatattcagaaggagatagacaggctgaatgaggttgccaagaacctcaacgagtctctgatcgatctgcaggagttgggcaagtacgaacagtatatcaaatggccatggtacatttggcttgggttcattgctgggctgatagctatcgtcatggtgacaattatgttgtgttgcatgacatcctgctgtagttgtctgaagggctgctgctcatgcggcagctgttgcgactacaaagacgatgacgacaagtaa

SEQ ID NO: 62 - protein, 1262 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRAAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCDYKDDDDK

SEQ ID NO: 63 - DNA, 357 bp, synthetic construct
  source 1..357: mol_type = other DNA
atgcaagtgcagctggtggaaaccggcggcggactcgtgcagcctggcggatctctgcggctgagctgtgccgcttctggctttacattcagcagcgtgtacatgaattgggtcagacaggcccctggaaaaggccctgagtgggtgtctagaatctccccaaacagcggcaacatcggctacaccgacagcgtgaagggcagattcaccatcagcagagataatgccaagaacaccctgtatctgcagatgaacaacctgaagcccgaggacaccgccctgtactactgcgccatcggcctgaacctgagctccagcagcgtgcggggccagggcacacaggttacagtgtccagc

SEQ ID NO: 64 - protein, 119 aa, synthetic construct
MQVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSS

SEQ ID NO: 65 - DNA, 387 bp, synthetic construct
  source 1..387: mol_type = other DNA
atgcaagtgcagctggtggaaagcggcggaggcctgatgcaggctggcggcagcctcagactgagctgcgccgtcagcggccggaccttctctacagccgccatgggctggtttagacaggcccctggcaaggaacgcgagttcgtggccgctatccggtggtccggcggatctgcctactacgctgatagcgtgaagggcagattcaccatcagcagagataaggccaagaacaccgtgtacctgcagatgaacagcctgaaatacgaggacaccgccgtgtactattgtgcccagacccactacgtgtcctacctgctgagcgactacgccacctggccctacgactactggggacagggcacacaggttacagtgtctagcaag

SEQ ID NO: 66 - protein, 129 aa, synthetic construct
MQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTHYVSYLLSDYATWPYDYWGQGTQVTVSSK

SEQ ID NO: 67 - protein, 120 aa, synthetic construct
GSQVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSS

SEQ ID NO: 68 - DNA, 789 bp, synthetic construct
  source 1..789: mol_type = other DNA
atggaccctgctgaggccgtgctgcaagagaaggccctgaaattcatgaacagcagcgagcgggaagattgcaacaacggcgaacctcctagaaagatcatccccgagaagaatagcctgagacagacctacaactcttgcgcccgcctctgtctgaaccaggagaccgtgtgcctggcttccacagctatgaaaaccgagaattgcgtggccaagaccaagcttgctaatggcacatctagcatgatcgtgcctaagcagagaaagctgtccgcctcttacgagaaggaaaaagaactgtgcgtcaagtacttcgagcagtggagcgagtctgatcaggtggaattcgtggaacacctgatcagccagatgtgccactaccagcacggccacatcaacagctacctgaagcctatgctgcagagagacttcatcaccgccctgcctgccagaggcctggaccatatcgccgagaacatcctgagctacctggacgccaaaagcctgtgtgccgccgagctggtgtgcaaggagtggtatagagtgacaagcgatggaatgctgtggaagaagctgattgagagaatggtgcggaccgacagcctgtggcggggcctggccgaacggagaggctggggacagtacctgtttaagaacaagcccccagacggcaacgcccctccaaacagcttctacagagccctgtaccccaagatcatccaggacatcgaaaccatcgagagcaactggaggtgtggccggcactccctgcaaagaatccactgcaga

SEQ ID NO: 69 - protein, 389 aa, synthetic construct
MDPAEAVLQEKALKFMNSSEREDCNNGEPPRKIIPEKNSLRQTYNSCARLCLNQETVCLASTAMKTENCVAKTKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLQRIHCRPMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLQRIHCR

SEQ ID NO: 70 - protein, 130 aa, synthetic construct
GSQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTHYVSYLLSDYATWPYDYWGQGTQVTVSSK